(19) United States
(12) Patent Application Publication
(10) Pub. No.: US 2013/0345392 A1
(43) Pub. Date: Dec. 26, 2013

(54) EDN3-LIKE PEPTIDES AND USES THEREOF

(71) Applicants: Tamas Bartfai, San Diego, CA (US); Bruno Conti, San Diego, CA (US); Avijit Ghosh, New Haven, CT (US); John Hadcock, Dedham, MA (US); Olivia Osborn, Solana Beach, CA (US); Manuel Sanchez-Alavez, San Diego, CA (US); Jean Schaefer, Niantic, CT (US); Iustin Tabarean, San Diego, CA (US)

(72) Inventors: Tamas Bartfai, San Diego, CA (US); Bruno Conti, San Diego, CA (US); Avijit Ghosh, New Haven, CT (US); John Hadcock, Dedham, MA (US); Olivia Osborn, Solana Beach, CA (US); Manuel Sanchez-Alavez, San Diego, CA (US); Jean Schaefer, Niantic, CT (US); Iustin Tabarean, San Diego, CA (US)

(73) Assignee: Pfizer Inc, New York, NY (US)

(21) Appl. No.: 14/002,283

(22) PCT Filed: Mar. 1, 2012
(86) PCT No.: PCT/IB12/50978

§ 371 Date: Aug. 29, 2013

Related U.S. Application Data
(60) Provisional application No. 61/449,465, filed on Mar. 4, 2011.

(51) Int. Cl. C07K 14/47

(52) U.S. Cl. CPC C07K 14/47
USPC 530/324

(57) ABSTRACT

This application discloses novel EDN3-like polypeptides. One such short polypeptide is EDN3 97-140, which is a 44 amino acid peptide that stimulates GLP-1 secretion in enteric cells and inhibits gluconeogenesis in hepatic cells. EDN3 97-140, as well as other EDN3-like polypeptides provided herein may be used in the study and treatment of a number of indications, including the treatment of metabolic disorders such as obesity and diabetes.

### CROSS-REFERENCE TO RELATED APPLICATION

This application claims the benefit of U.S. Provisional Application No. 61/449,465, filed Mar. 4, 2011. The entire teachings of the referenced application are expressly incorporated herein by reference.

### BACKGROUND

Peptide ligands are processed from larger propeptides, and a single gene product can yield numerous bioactive products by alternative processing. For instance, glucagon gives rise to eight peptide hormones (see Hoist, 2007) with a variety of well described functions in different tissues, such as oxyntomodulin, glucagon, GLP-1, and GLP-2. Similarly, Endothelin-3 is a vasoactive peptide derived from a longer precursor, preproendothelin-3 (Bloch, 1989). Endothelin-3 is a member of the endothelin family originally cloned from the human hypothalamus (Bloch, 1989). The endothelins (1-3) have similar amino acid sequences across 21 amino acids making up the well characterized mature peptides after processing from preproendothelin (Inoue, 1989). However, they remain pharmacologically distinct.

This application discloses novel polypeptides having yet another distinct activity: anti-hyperglycemic activity. The present disclosure provides polypeptides referred to as EDN3-like polypeptides.

### SUMMARY

The present disclosure provides polypeptides referred to as EDN3-like polypeptides. Exemplary EDN3-like polypeptides, such as EDN3 97-140, are provided herein. EDN3-like polypeptides include variants of EDN3 97-140, as described herein. The present disclosure also provides methods of identifying suitable EDN3-like polypeptides, and methods of using said polypeptides in vitro, ex vivo, and in vivo, including in the study and treatment of disease. The present disclosure also provides methods for identifying the one or more receptors that mediate the effects of EDN3-like polypeptides. The disclosure contemplates polypeptides, including isolated or purified polypeptides, comprising or consisting of any of the END3-like polypeptides provided herein, as well as methods for using any such polypeptides. It is noted that although the disclosure provides certain functional attributes for END3-like polypeptides, suitable polypeptides may be described and provided with or without any reference to such functional attributes. For instance, in certain embodiments, the EDN3-like polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes, and in certain embodiments, these characteristics or describing the polypeptides using these characteristics is optional.

In certain aspects, the present disclosure provides an isolated polypeptide comprising or consisting of the amino acid sequence of any of SEQ ID No. 1-33. In certain embodiments, the isolated polypeptide is less than or equal to 60 amino acid residues. In certain embodiments, the isolated polypeptide is provided as a fusion protein or conjugate with an additional heterologous protein or a label. For any of SEQ ID No. 1-33, it is understood that any residue that is permitted to vary (e.g., indicated with an X) can vary as described herein or as indicated by the Examples. As with all other EDN3-like polypeptides described, the disclosure contemplates that any such polypeptides may be used in any of the methods described herein. Further exemplary features of the disclosure are described below.

In certain aspects, the present disclosure provides an isolated polypeptide consisting of: (i) the amino acid sequence CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii); wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In certain aspects, the present disclosure also provides an isolated polypeptide consisting of: (i) the amino acid sequence CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii); wherein the polypeptide is capable of one or more of: inhibiting glucose production in hepatocytes, promoting GLP-1 secretion in the rat perfused colon assay, or promoting GLP-1 secretion in GLUTag cells.

In certain aspects, the present disclosure provides a polypeptide comprising: (a) a first polypeptide portion consisting of (i) the amino acid sequence CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii), and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In certain aspects, the present disclosure also provides a polypeptide comprising: (a) a first polypeptide portion consisting of (i) the amino acid sequence CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii), and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, or promoting GLP-1 secretion in GLUTag cells.

In certain embodiments, the substitution is a conservative substitution. In certain embodiments, the polypeptide consists of an amino acid sequence CTCFTYKDKECVYYCHLDIIWINTPEQTVPYGLSNYRES (SEQ ID No. 26), or an amino acid sequence having 1, 2, or 3 substitutions relative to SEQ ID No. 26. In certain embodiments, the first polypeptide portion consists of an amino acid sequence CTCFTYKDKECVYYCHLDIIWINTPEQTVPYGLSNYRES (SEQ ID No. 26), or an amino acid sequence having 1, 2, or 3 substitutions relative to SEQ ID No. 26. In certain embodiments, the polypeptide consists of an amino acid sequence having 1, 2, or 3 substitutions relative to the fragment of (i) beginning at position 1 and ending at position 27 of SEQ ID No. 1. In certain embodiments, the polypeptide consists of the fragment of (i) beginning at position 1 and ending at position 27 of SEQ ID No. 1. In certain embodiments, the first polypeptide portion consists of an amino acid sequence having 1, 2, or 3 substitutions relative to the fragment of (i) beginning at position 1 and ending at position 27 of SEQ ID No. 1. In certain embodiments, the first polypeptide portion consists of the fragment of (i) beginning at position 1 and ending at position 27 of SEQ ID No. 1. In certain embodiments, the polypeptide consists of an amino acid sequence having 1, 2, or 3 substitutions relative to the fragment of (i) beginning at position 1 and ending at position 31 of SEQ ID No. 1. In certain embodiments, the polypeptide consists of the fragment of (i) beginning at position 1 and ending at position 31 of SEQ ID No. 1. In certain embodiments, the first polypeptide portion consists of an amino acid sequence having 1, 2, or 3 substitutions relative to the fragment of (i) beginning at position 1 and ending at position 31 of SEQ ID No. 1. In certain embodiments, the first polypeptide portion consists of the fragment of (i) beginning at position 1 and ending at position 31 of SEQ ID No. 1. In certain embodiments, the polypeptide includes said 1, 2, or 3 substitutions. In certain embodiments, the polypeptide does not include said 1, 2, or 3 substitutions. In certain embodiments, positions 1, 3, 11, and 15 of SEQ ID No. 1 are each C, and a first disulfide bridge connects the cysteine at position 1 of SEQ ID No. 1 with the cysteine at position 15 of SEQ ID No. 1, and a second disulfide bridge connects the cysteine at position 3 of SEQ ID No. 1 with the cysteine at position 11 of SEQ ID No. 1.

In certain aspects, the present disclosure also provides an isolated polypeptide consisting of: (i) the amino acid sequence X₁TX₂FTYKDKEX₃VYYX₄HLDIIX₅INTPEQTVPYGLSNYRX₆SX₇ (SEQ ID No. 2) or (ii) a fragment of (i) beginning at position 1 and ending at any one of positions 27 to 39 of SEQ ID No. 2; wherein X₁, X₂, X₃, X₄, X₅, X₆, and X₇ are independently selected from any amino acid; and wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In certain other aspects, the present disclosure also provides an isolated polypeptide consisting of: (i) the amino acid sequence X₁TX₂FTYKDKEX₃VYYX₄HLDIIX₅INTPEQTVPYGLSNYRX₆SX₇ (SEQ ID No. 2) or (ii) a fragment of (i) beginning at position 1 and ending at any one of positions 27 to 39 of SEQ ID No. 2; wherein X₁, X₂, X₃, X₄, X₅, X₆, and X₇ are independently selected from any amino acid; and wherein the polypeptide is capable of one or more of: inhibiting glucose production in hepatocytes, promoting GLP-1 secretion in the rat perfused colon assay, or promoting GLP-1 secretion in GLUTag cells.

In certain aspects, the present disclosure also provides a polypeptide comprising: (a) a first polypeptide portion consisting of: (i) X₁TX₂FTYKDKEX₃VYYX₄HLDIIX₅INTPEQTVPYGLSNYRX₆SX₇ (SEQ ID No. 2) or (ii) a fragment of (i) beginning at position 1 and ending at any one of positions 27 to 39 SEQ ID No. 2 and (b) a second portion, which polypeptide portion is a polypeptide portion heterologous to said first polypeptide portion or is a detectable label; wherein X₁, X₂, X₃, X₄, X₅, X₆, and X₇ are independently selected from any amino acid; and wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In certain aspects, the present disclosure also provides a polypeptide comprising: (a) a first polypeptide portion consisting of: (i) X₁TX₂FTYKDKEX₃VYYX₄HLDIIX₅INTPEQTVPYGLSNYRX₆SX₇ (SEQ ID No. 2) or (ii) a fragment of (i) beginning at position 1 and ending at any one of positions 27 to 39 SEQ ID No. 2 and (b) a second portion, which polypeptide portion is a polypeptide portion heterologous to said first polypeptide portion or is a detectable label; wherein X₁, X₂, X₃, X₄, X₅, X₆, and X₇ are independently selected from any amino acid; and wherein the polypeptide is capable of one or more of: inhibiting glucose production in hepatocytes, promoting GLP-1 secretion in the rat perfused colon assay, or promoting GLP-1 secretion in GLUTag cells.

In some embodiments, X₁, X₂, X₃, X₄, X₅, X₆, and X₇ are independently selected from the corresponding position in SEQ ID NO: 19 or SEQ ID NO: 21 or a conservative substitution thereof. In some embodiments, the polypeptide consists of an amino acid sequence X₁TX₂FTYKDKEX₃VYYX₄HLDIIX₅INTPEQTVPYGLSNYRX₆S (SEQ ID No. 27), wherein X₁, X₂, X₃, X₄, X₅, and X₆ are independently selected from any amino acid. In some embodiments, the first polypeptide portion consists of an amino acid sequence X₁TX₂FTYKDKEX₃VYYX₄HLDIIX₅INTPEQTVPYGLSNYRX₆S (SEQ ID No. 27), wherein X₁, X₂, X₃, X₄, X₅, and X₆ are independently selected from any amino acid. In some embodiments, the polypeptide consists of the fragment of (i) beginning at position 1 and ending at position 27 of SEQ ID No. 2. In some embodiments, the first polypeptide portion consists of the fragment of (i) beginning at position 1 and ending at position 27 of SEQ ID No. 2. In some embodiments, the polypeptide consists of the fragment of (i) beginning at position 1 and ending at position 31 of SEQ ID No. 2. In some embodiments, the first polypeptide portion consists of the fragment of (i) beginning at position 1 and ending at position 31 of SEQ ID No. 2.

In some embodiments, X₁ is C or S. In some embodiments, X₂ is C or S. In some embodiments, X₃ is C or S. In some embodiments, X₄ is C, S, or A. In some embodiments, X₅ is W or A. In some embodiments, X₆ is G or S. In some embodiments, X₇ is L or F. In some embodiments, X₁, X₂, X₃, and X₄ are each C. In some embodiments, a first disulfide bridge connects the cysteine at position 1 of SEQ ID No. 2 with the cysteine at position 15 of SEQ ID No. 2, and a second disulfide bridge connects the cysteine at position 3 of SEQ ID No. 2 with the cysteine at position 11 of SEQ ID No. 2.

In some aspects, this disclosure provides an isolated polypeptide of less than or equal to 60 amino acids, wherein the polypeptide comprises the amino acid sequence YYSHLDIIWINTPEQ (SEQ ID No. 3) or YYAHLDIIAINTPEQ (SEQ ID No. 4); wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes. In some aspects, this disclosure provides an isolated polypeptide of less than or equal to 60 amino acids, wherein the polypeptide comprises the amino acid sequence YYSHLDIIWINTPEQ (SEQ ID No. 3) or YYAHLDIIAINTPEQ (SEQ ID No. 4); wherein the polypeptide is capable of one or more of: inhibiting glucose production in hepatocytes, promoting GLP-1 secretion in the rat perfused colon assay, or promoting GLP-1 secretion in GLUTag cells.

In some aspects, this disclosure provides a polypeptide comprising: (a) a first polypeptide portion comprising the amino acid sequence YYSHLDIIWINTPEQ (SEQ ID No. 3) or YYAHLDIIAINTPEQ (SEQ ID No. 4), but which first polypeptide portion is less than or equal to 60 amino acid residues in length and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes. In some aspects, this disclosure provides a polypeptide comprising: (a) a first polypeptide portion comprising the amino acid sequence YYSHLDIIWINTPEQ (SEQ ID No. 3) or YYAHLDIIAINTPEQ (SEQ ID No. 4), but which first polypeptide portion is less than or equal to 60 amino acid residues in length and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, or promoting GLP-1 secretion in GLUTag cells.

In some embodiments, the polypeptide comprises the amino acid sequence YYSHLDIIWINTPEQTVPYGLSNYRX₆SX₇R (SEQ ID No. 5) or YYAHLDIIAINTPEQTVPYGLSNYRX₆SX₇R (SEQ ID No. 6); wherein X₆ and X₇ are independently selected from any amino acid. In some embodiments, the polypeptide comprises the amino acid sequence YYSHLDIIWINTPEQTVPYGLSNYRX₆SX₇RGKR (SEQ ID No. 22) or YYAHLDIIAINTPEQTVPYGLSNYRX₆SX₇RGKR (SEQ ID No. 23); wherein X₆ and X₇ are independently selected from any amino acid. In some embodiments, the first polypeptide portion comprises the amino acid sequence YYSHLDIIWINTPEQTVPYGLSNYRX₆SX₇R (SEQ ID No. 5) or YYAHLDIIAINTPEQTVPYGLSNYRX₆SX₇R (SEQ ID No. 6); wherein X₆ and X₇ are independently selected from any amino acid. In some embodiments, the first polypeptide portion comprises the amino acid sequence YYSHLDIIWINTPEQTVPYGLSNYRX₆SX₇RGKR (SEQ ID No. 22) or YYAHLDIIAINTPEQTVPYGLSNYRX₆SX₇RGKR (SEQ ID No. 23); wherein X₆ and X₇ are independently selected from any amino acid. In some embodiments, X₆ is G or S. In some embodiments, X₇ is L or F.

In certain aspects, this disclosure provides an isolated polypeptide consisting of: (i) a fragment of the amino acid sequence CTCFTYKDKECVYYCHLDIIWINTPEQTVPYGLSNYRGSFRGKR (SEQ ID No. 19) of 29 to 41 contiguous amino acids, said fragment beginning at any one of positions 4 to 13 and ending at any one of positions 41 to 44 of SEQ ID No. 19, or (ii) an amino acid sequence having 1, 2, or 3 substitutions relative to said fragment; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes. Furthermore, in certain aspects, this disclosure provides a polypeptide comprising: (a) a first polypeptide portion consisting of: (i) a fragment of the amino acid sequence CTCFTYKDKECVYYCHLDIIWINTPEQTVPYGLSNYRGSFRGKR (SEQ ID No. 19) of 29 to 41 contiguous amino acids, said fragment beginning at any one of positions 4 to 13 and ending at any one of positions 41 to 44 of SEQ ID No. 19, or (ii) an amino acid sequence having 1, 2, or 3 substitutions relative to said fragment, and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In some embodiments, the substitution is a conservative substitution.

In certain aspects, this disclosure provides an isolated polypeptide consisting of: (i) a fragment of the amino acid sequence CTCFTYKDKECVYYCHLDIIWINTPEQTVPYGLSNYRGSFRGKR (SEQ ID No. 19) of 29 to 41 contiguous amino acids, said fragment beginning at any one of positions 4 to 13 and ending at any one of positions 41 to 44 of SEQ ID No. 19, or (ii) an amino acid sequence having 1, 2, or 3 substitutions relative to said fragment; wherein the polypeptide is capable of one or more of: inhibiting glucose production in hepatocytes, promoting GLP-1 secretion in the rat perfused colon assay, or promoting GLP-1 secretion in GLUTag cells. Furthermore, in certain aspects, this disclosure provides a polypeptide comprising: (a) a first polypeptide portion consisting of: (i) a fragment of the amino acid sequence CTCFTYKDKECVYYCHLDIIWINTPEQTVPYGLSNYRGSFRGKR (SEQ ID No. 19) of 29 to 41 contiguous amino acids, said fragment beginning at any one of positions 4 to 13 and ending at any one of positions 41 to 44 of SEQ ID No. 19, or (ii) an amino acid sequence having 1, 2, or 3 substitutions relative to said fragment, and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, or promoting GLP-1 secretion in GLUTag cells.

In some aspects, the disclosure also provides an isolated polypeptide consisting of a fragment of the amino acid sequence X₁TX₂FTYKDKEX₃VYYX₄HLDIIX₅INTPEQTVPYGLSNYRX₆SX₇RGKR (SEQ ID No. 20) of 29 to 41 contiguous amino acids, said fragment beginning at any one of positions 4 to 13 and ending at any one of positions 41 to 44 of SEQ ID No. 20, wherein X₃, X₄, X₅, X₆, and X₇ are independently selected from any amino acid; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes. Furthermore, in some aspects, the disclosure provides an isolated polypeptide consisting of a fragment of the amino acid sequence X₁TX₂FTYKDKEX₃VYYX₄HLDIIX₅INTPEQTVPYGLSNYRX₆SX₇RGKR (SEQ ID No. 20) of 29 to 41 contiguous amino acids, said fragment beginning at any one of positions 4 to 13 and ending at any one of positions 41 to 44 of SEQ ID No. 20, wherein X₃, X₄, X₅, X₆, and X₇ are independently selected from any amino acid; wherein the polypeptide is capable of one or more of: inhibiting glucose production in hepatocytes, promoting GLP-1 secretion in the rat perfused colon assay, or promoting GLP-1 secretion in GLUTag cells.

Moreover, in some aspects, the disclosure provides a polypeptide comprising: (a) a first polypeptide portion consisting of: a fragment of the amino acid sequence X₁TX₂FTYKDKEX₃VYYX₄HLDIIX₅INTPEQTVPYGLSNYRX₆SX₇RGKR (SEQ ID No. 20) of 29 to 41 contiguous amino acids, said fragment beginning at any one of positions 4 to 13 and ending at any one of positions 41 to 44 of SEQ ID No. 20, wherein X₃, X₄, X₅, X₆, and X₇ are independently selected from any amino acid, and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes. In some aspects, the disclosure provides a polypeptide comprising: (a) a first polypeptide portion consisting of: a fragment of the amino acid sequence X₁TX₂FTYKDKEX₃VYYX₄HLDIIX₅INTPEQTVPYGLSNYRX₆SX₇RGKR (SEQ ID No. 20) of 29 to 41 contiguous amino acids, said fragment beginning at any one of positions 4 to 13 and ending at any one of positions 41 to 44 of SEQ ID No. 20, wherein X₃, X₄, X₅, X₆, and X₇ are independently selected from any amino acid, and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, or promoting GLP-1 secretion in GLUTag cells.

In certain embodiments, X₁, X₂, X₃, X₄, X₅, X₆, and X₇ are independently selected from the corresponding position in SEQ ID NO: 19 or SEQ ID NO: 21 or a conservative substitution thereof. In certain embodiments, the first polypeptide portion is N-terminal to the second polypeptide portion. In certain embodiments, the first polypeptide portion is C-terminal to the second polypeptide portion. In certain embodiments, the polypeptide has a C-terminal moiety of —OH. In certain embodiments, the polypeptide is amidated at the C-terminus. In certain embodiments, the polypeptide is less than 60 amino acids in length.

In certain embodiments, the polypeptide is linked to a detectable label. In certain embodiments, the label is a radiolabel, a fluorescent label, or an MRI-detectable label. In certain embodiments, the radiolabel is ²H, ³H, ¹¹C, ¹³C, ¹⁴C, ¹⁵N, ¹⁸O, ¹⁷O, ¹⁸F, ³⁶Cl, ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb, ^{81m}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi or ²¹³Bi. In certain embodiments, the fluorescent label is Texas Red, phycoerythrin (PE), cytochrome c, Cy2, Cy3, Cy3.5, Cy5, Cy5.5, Cy7, fluorescent isothiocyante (FITC), tetramethylrhodamine isothiocyanate (TRITC), allophycocyanin (APC), an Alexa Fluor dye, a quantum dot dye, fluorescein, rhodamine, umbeliferone, DRAQ5, acridone, quinacridone, a lanthanide chelate, a ruthenium complexe, tartrazine, phycocyanin, or allophycocyanin. In certain embodiments, the MRI-detectable label comprises a paramagnetic imaging agent, superparamagnetic iron-oxide particles, magnetite particles, a fluorocarbon imaging reagent, a Gd chelate, or a Mn chelate.

In some aspects, this disclosure provides a compound comprising an EDN3-like polypeptide linked to a detectable label.

In certain embodiments, the second polypeptide portion comprises: (i) a constant region from an IgG heavy chain, (ii) an Fc domain, (iii) purification sequence selected from: an epitope tag, a FLAG tag, a polyhistidine sequence, and a GST fusion, or (iv) a signal sequence.

In certain embodiments, the second polypeptide portion does not encode a polypeptide that is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In certain embodiments, the polypeptide is an isolated polypeptide. In certain embodiments, the second portion is a detectable label.

In certain embodiments, the polypeptide is a peptidomimetic.

In certain embodiments, the polypeptide does not bind to endothelin receptor A (ET_{A}). In certain embodiments, the polypeptide does not bind to endothelin receptor B (ET_{B}).

In certain aspects, this disclosure provides a composition comprising any of the polypeptides herein, formulated with a pharmaceutically acceptable carrier. In some embodiments, the composition is substantially pyrogen-free.

In certain aspects, this disclosure provides a composition suitable for administration to a human or animal subject, comprising a polypeptide consisting of: (i) the amino acid sequence CTCFTYKDKECVYYCHLDIIWINTPEQTVPYGLSNYRGSFRGKR (SEQ ID No. 19), or (ii) a fragment of (i) beginning at position 1 and ending at any one of positions 41 to 43 of SEQ ID No. 19; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes; formulated with a pharmaceutically acceptable carrier, which composition is substantially non-pyrogenic. In certain aspects, this disclosure provides a composition suitable for administration to a human or animal subject, comprising a polypeptide consisting of: (i) the amino acid sequence CTCFTYKDKECVYYCHLDIIWINTPEQTVPYGLSNYRGSFRGKR (SEQ ID No. 19), or (ii) a fragment of (i) beginning at position 1 and ending at any one of positions 41 to 43 of SEQ ID No. 19; wherein the polypeptide is capable of one or more of: inhibiting glucose production in hepatocytes, promoting GLP-1 secretion in the rat perfused colon assay, or promoting GLP-1 secretion in GLUTag cells; formulated with a pharmaceutically acceptable carrier, which composition is substantially non-pyrogenic.

In certain aspects, this disclosure provides a composition comprising a polypeptide consisting of: (i) the amino acid sequence CTCFTYKDKECVYYCHLDIIWINTPEQTVPYGLSNYRESLRGKR (SEQ ID No. 21) or (ii) a fragment of (i) beginning at position 1 and ending at any one of positions 41 to 43 of SEQ ID No. 21; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes; formulated with a pharmaceutically acceptable carrier, which composition is substantially non-pyrogenic. In certain aspects, this disclosure provides a composition comprising a polypeptide consisting of: (i) the amino acid sequence CTCFTYKDKECVYYCHLDIIWINTPEQTVPYGLSNYRESLRGKR (SEQ ID No. 21) or (ii) a fragment of (i) beginning at position 1 and ending at any one of positions 41 to 43 of SEQ ID No. 21; wherein the polypeptide is capable of one or more of: inhibiting glucose production in hepatocytes, promoting GLP-1 secretion in the rat perfused colon assay, or promoting GLP-1 secretion in GLUTag cells; formulated with a pharmaceutically acceptable carrier, which composition is substantially non-pyrogenic.

In certain aspects, this disclosure provides a composition suitable for administration to a human or animal subject, comprising a polypeptide consisting of: (a) a first polypeptide portion consisting of (i) the amino acid sequence CTCFTYKDKECVYYCHLDIIWINTPEQTVPYGLSNYRGSFRGKR (SEQ ID No. 19), or (ii) a fragment of (i) beginning at position 1 and ending at any one of positions 41 to 43 of SEQ ID No. 19; and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes; formulated with a pharmaceutically acceptable carrier, which composition is substantially non-pyrogenic. In certain aspects, this disclosure provides a composition suitable for administration to a human or animal subject, comprising a polypeptide consisting of: (a) a first polypeptide portion consisting of (i) the amino acid sequence CTCFTYKDKECVYYCHLDIIWINTPEQTVPYGLSNYRGSFRGKR (SEQ ID No. 19), or (ii) a fragment of (i) beginning at position 1 and ending at any one of positions 41 to 43 of SEQ ID No. 19; and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, or promoting GLP-1 secretion in GLUTag cells; formulated with a pharmaceutically acceptable carrier, which composition is substantially non-pyrogenic.

In certain aspects, this disclosure provides a composition comprising a polypeptide consisting of: (a) a first polypeptide portion consisting of (i) the amino acid sequence CTCFTYKDKECVYYCHLDIIWINTPEQTVPYGLSNYRESLRGKR (SEQ ID No. 21) or (ii) a fragment of (i) beginning at position 1 and ending at any one of positions 41 to 43 of SEQ ID No. 21; and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes; formulated with a pharmaceutically acceptable carrier, which composition is substantially non-pyrogenic. In certain aspects, this disclosure provides a composition comprising a polypeptide consisting of: (a) a first polypeptide portion consisting of (i) the amino acid sequence CTCFTYKDKECVYYCHLDIIWINTPEQTVPYGLSNYRESLRGKR (SEQ ID No. 21) or (ii) a fragment of (i) beginning at position 1 and ending at any one of positions 41 to 43 of SEQ ID No. 21; and (b) a second portion, which second portion is a polypeptide portion heterologous to said first polypeptide portion or is a detectable label; wherein the polypeptide is capable of one or more of: inhibiting glucose production in hepatocytes, promoting GLP-1 secretion in the rat perfused colon assay, or promoting GLP-1 secretion in GLUTag cells; formulated with a pharmaceutically acceptable carrier, which composition is substantially non-pyrogenic.

In some embodiments, the composition further comprises an anti-diabetic agent. In some embodiments, the composition further comprises an anti-obesity agent.

In certain aspects, this disclosure provides an isolated nucleic acid comprising: (a) a first nucleic acid portion consisting of a sequence encoding an EDN-3 like polypeptide as described herein, and (b) a second nucleic acid portion, which second nucleic acid portion is heterologous to said first nucleic acid portion; wherein the amino acid sequence of part (a) is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes. In certain aspects, this disclosure provides an isolated nucleic acid comprising: (a) a first nucleic acid portion consisting of a sequence encoding an EDN-3 like polypeptide as described herein, and (b) a second nucleic acid portion, which second nucleic acid portion is heterologous to said first nucleic acid portion; wherein the amino acid sequence of part (a) is capable of one or more of: inhibiting glucose production in hepatocytes, promoting GLP-1 secretion in the rat perfused colon assay, or promoting GLP-1 secretion in GLUTag cells.

In some embodiments, the first nucleic acid portion does not encode an amino acid sequence greater than 44 amino acids in length. In some embodiments, the isolated nucleic acid does not encode a polypeptide greater than 60 amino acids in length. In some embodiments, the first nucleic acid portion is upstream of the second nucleic acid portion. In some embodiments, the first nucleic acid portion is downstream of the second nucleic acid portion. In some embodiments, the isolated nucleic acid encodes a fusion protein. In some embodiments, the second nucleic acid portion is a promoter sequence. In some embodiments, the second nucleic acid portion is a selectable marker.

In certain aspects, the disclosure also provides an expression vector comprising an isolated nucleic acid encoding an EDN3-like polypeptide, as described herein. In some embodiments, the nucleic acid is operably linked to a heterologous promoter sequence.

In certain aspects, the disclosure also provides a host cell comprising an expression vector encoding an EDN3-like polypeptide, as described herein. In certain aspects, the disclosure also provides a host cell comprising a nucleic acid, as described herein.

In certain aspects, this disclosure provides a method of producing an EDN3-like polypeptide, as described herein, comprising: (a) providing a cell comprising a nucleic acid that encodes said polypeptide, and (b) culturing the cell under conditions that allow the production of said polypeptide. In some embodiments, the method further comprises a step of (c) isolating the polypeptide.

In certain aspects, this disclosure provides a method of producing an EDN3-like polypeptide, comprising chemically synthesizing said polypeptide. In some embodiments, the method further comprises amidating said polypeptide at the C-terminal amino acid.

In certain aspects, this disclosure provides a method of treating a metabolic disease or disorder, comprising administering to a subject in need thereof an effective amount of the EDN3-like polypeptide.

In certain aspects, this disclosure provides a method of treating a metabolic disease or disorder, comprising administering to a subject in need thereof an effective amount of the composition comprising an EDN3-like polypeptide.

In certain aspects, this disclosure provides a method of treating a metabolic disease or disorder, comprising administering to a subject in need thereof an effective amount of an isolated polypeptide of less than or equal to 60 amino acids in length, wherein the polypeptide comprises the amino acid sequence YYCHLDIIWINTPEQ (SEQ ID No. 7) or an amino acid sequence having 1, 2, or 3 substitutions relative to SEQ ID No. 7, wherein the polypeptide does not include gastric inhibitory peptide.

In certain aspects, this disclosure provides a method of treating a metabolic disease or disorder, comprising administering to a subject in need thereof an effective amount of a polypeptide comprising (a) a first polypeptide portion comprising the amino acid sequence YYCHLDIIWINTPEQ (SEQ ID No. 7) or an amino acid sequence having 1, 2, or 3 substitutions relative to SEQ ID No. 7, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In some embodiments, the substitution is a conservative substitution. In some embodiments, the polypeptide comprises the amino acid sequence YKDKECVYYCHLDIIWINTPEQ (SEQ ID No. 24), or an amino acid sequence having 1, 2, or 3 substitutions relative to SEQ ID No. 24. In some embodiments, the first polypeptide portion comprises an amino acid sequence YKDKECVYYCHLDIIWINTPEQ (SEQ ID No. 24), or an amino acid sequence having 1, 2, or 3 substitutions relative to SEQ ID No. 24. In some embodiments, the polypeptide comprises the amino acid sequence CTCFTYKDKECVYYCHLDIIWINTPEQ (SEQ ID No. 9), or an amino acid sequence having 1, 2, or 3 substitutions relative to SEQ ID No. 9. In some embodiments, the first polypeptide portion comprises an amino acid sequence CTCFTYKDKECVYYCHLDIIWINTPEQ (SEQ ID No. 9), or an amino acid sequence having 1, 2, or 3 substitutions relative to SEQ ID No. 9.

In some embodiments, the polypeptide comprises the amino acid sequence CTCFTYKDKECVYYCHLDIIWINTPEQTVPY (SEQ ID No. 11), or an amino acid sequence having 1, 2, or 3 substitutions relative to SEQ ID No. 11. In some embodiments, the first polypeptide portion comprises an amino acid sequence CTCFTYKDKECVYYCHLDIIWINTPEQTVPY (SEQ ID No. 11), or an amino acid sequence having 1, 2, or 3 substitutions relative to SEQ ID No. 11. In some embodiments, the polypeptide comprises the amino acid sequence CTCFTYKDKECVYYCHLDIIWINTPEQTVPYGLSNYRGSFR (SEQ ID No. 13), or an amino acid sequence having 1, 2, or 3 substitutions relative to SEQ ID No. 13. In some embodiments, the first polypeptide portion comprises an amino acid sequence CTCFTYKDKECVYYCHLDIIWINTPEQTVPYGLSNYRGSFR (SEQ ID No. 13), or an amino acid sequence having 1, 2, or 3 substitutions relative to SEQ ID No. 13.

In some embodiments, the polypeptide comprises the amino acid sequence YYCHLDIIWINTPEQTVPYGLSNYRGSFR (SEQ ID No. 15), or an amino acid sequence having 1, 2, or 3 substitutions relative to SEQ ID No. 15. In some embodiments, the first polypeptide portion comprises an amino acid sequence YYCHLDIIWINTPEQTVPYGLSNYRGSFR (SEQ ID No. 15), or an amino acid sequence having 1, 2, or 3 substitutions relative to SEQ ID No. 15. In some embodiments, the polypeptide comprises the amino acid sequence YYCHLDIIWINTPEQTVPYGLSNYRGSFRGKR (SEQ ID No. 17), or an amino acid sequence having 1, 2, or 3 substitutions relative to SEQ ID No. 17. In some embodiments, the first polypeptide portion comprises an amino acid sequence YYCHLDIIWINTPEQTVPYGLSNYRGSFRGKR (SEQ ID No. 17), or an amino acid sequence having 1, 2, or 3 substitutions relative to SEQ ID No. 17.

In some embodiments, the polypeptide comprises the amino acid sequence CTCFTYKDKECVYYCHLDIIWINTPEQTVPYGLSNYRGSFRGKR (SEQ ID No. 19), or an amino acid sequence having 1, 2, or 3 substitutions relative to SEQ ID No. 19. In some embodiments, the first polypeptide portion comprises an amino acid sequence CTCFTYKDKECVYYCHLDIIWINTPEQTVPYGLSNYRGSFRGKR (SEQ ID No. 19), or an amino acid sequence having 1, 2, or 3 substitutions relative to SEQ ID No. 19. In some embodiments, the polypeptide comprises the amino acid sequence CTCFTYKDKECVYYCHLDIIWINTPEQTVPYGLSNYRGS (SEQ ID No. 28), or an amino acid sequence having 1, 2, or 3 substitutions relative to SEQ ID No. 28. In some embodiments, the first polypeptide portion comprises an amino acid sequence CTCFTYKDKECVYYCHLDIIWINTPEQTVPYGLSNYRGS (SEQ ID No. 28), or an amino acid sequence having 1, 2, or 3 substitutions relative to SEQ ID No. 28. In some embodiments, the polypeptide includes said 1, 2, or 3 substitutions. In some embodiments, the polypeptide does not include said 1, 2, or 3 substitutions.

In certain aspects, this disclosure provides a method of treating a metabolic disease or disorder, comprising administering to a subject in need thereof an effective amount of an isolated polypeptide of less than or equal to 60 amino acids in length, wherein the polypeptide comprises the amino acid sequence YYX₄HLDIIX₅INTPEQ (SEQ ID No. 8), wherein X₄ and X₅ are independently selected from any amino acid, wherein the polypeptide does not include gastric inhibitory peptide.

In certain aspects, this disclosure provides a method of treating a metabolic disease or disorder, comprising administering to a subject in need thereof an effective amount of a polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In some embodiments, X₄ and X₅ are independently selected from the corresponding position in SEQ ID NO: 19 or SEQ ID NO: 21 or a conservative substitution thereof. In some embodiments, the polypeptide comprises the amino acid sequence YKDKEX₃VYYX₄HLDIIX₅INTPEQ (SEQ ID No. 25), and wherein X₃, X₄ and X₅ are independently selected from any amino acid. In some embodiments, the first polypeptide portion comprises the amino acid sequence YKDKEX₃VYYX₄HLDIIX₅INTPEQ (SEQ ID No. 25), and wherein X₃, X₄ and X₅ are independently selected from any amino acid. In some embodiments, the polypeptide comprises the amino acid sequence X₁TX₂FTYKDKEX₃VYYX₄HLDIIX₅INTPEQT (SEQ ID No. 33), wherein: X₁ and X₄ are C and X₂ and X₃ are independently selected from any amino acid, or X₂ and X₃ are C and X₁ and X₄ are independently selected from any amino acid, and X₅ is any amino acid. In some embodiments, the polypeptide comprises the amino acid sequence X₁TX₂FTYKDKEX₃VYYX₄HLDIIX₅INTPEQTVPYGLSNYRX₆SX₇R (SEQ ID No. 14), wherein: X₁ and X₄ are C and X₂ and X₃ are A, or X₂ and X₃ are C and X₁ and X₄ are A, X₅ is W, F, or Y, X₆ is E or G, and X₇ is L or F. In some embodiments, the polypeptide comprises the amino acid sequence X₁TX₂FTYKDKEX₃VYYX₄HLDIIX₅INTPEQ (SEQ ID No. 10), and wherein X₁, X₂, X₃, X₄, and X₅ are independently selected from any amino acid. In some embodiments, the first polypeptide portion comprises the amino acid sequence X₁TX₂FTYKDKEX₃VYYX₄HLDIIX₅INTPEQ (SEQ ID No. 10), and wherein X₁, X₂, X₃, X₄, and X₅ are independently selected from any amino acid.

In some embodiments, the polypeptide comprises the amino acid sequence X₁TX₂FTYKDKEX₃VYYX₄HLDIIX₅INTPEQTVPY (SEQ ID No. 12), and wherein X₁, X₂, X₃, X₄, and X₅ are independently selected from any amino acid. In some embodiments, the first polypeptide portion comprises the amino acid sequence X₁TX₂FTYKDKEX₃VYYX₄HLDIIX₅INTPEQTVPY (SEQ ID No. 12), and wherein X₁, X₂, X₃, X₄, and X₅ are independently selected from any amino acid. In some embodiments, the polypeptide comprises the amino acid sequence X₁TX₂FTYKDKEX₃VYYX₄HLDIIX₅INTPEQTVPYGLSNYRX₆SX₇R (SEQ ID No. 14), and wherein X₁, X₂, X₃, X₄, X₅, X₆, and X₇ are independently selected from any amino acid. In some embodiments, the first polypeptide portion comprises the amino acid sequence X₁TX₂FTYKDKEX₃VYYX₄HLDIIX₅INTPEQTVPYGLSNYRX₆SX₇R (SEQ ID No. 14), and wherein X₁, X₂, X₃, X₄, X₅, X₆, and X₇ are independently selected from any amino acid. In some embodiments, the polypeptide comprises the amino acid sequence YYX₄HLDIIX₅INTPEQTVPYGLSNYRX₆SX₇R (SEQ ID No. 16), and wherein X₄, X₅, X₆ and X₇ are independently selected from any amino acid. In some embodiments, the first polypeptide portion comprises the amino acid sequence YYX₄HLDIIX₅INTPEQTVPYGLSNYRX₆SX₇R (SEQ ID No. 16), and wherein X₄, X₅, X₆ and X₇ are independently selected from any amino acid.

In some embodiments, the polypeptide comprises the amino acid sequence YYX₄HLDIIX₅INTPEQTVPYGLSNYRX₆SX₇RGKR (SEQ ID No. 18), and wherein X₄, X₅, X₆ and X₇ are independently selected from any amino acid. In some embodiments, the first polypeptide portion comprises the amino acid sequence YYX₄HLDIIX₅INTPEQTVPYGLSNYRX₆SX₇RGKR (SEQ ID No. 18), and wherein X₄, X₅, X₆ and X₇ are independently selected from any amino acid. In some embodiments, the polypeptide comprises the amino acid sequence X₁TX₂FTYKDKEX₃VYYX₄HLDIIX₅INTPEQTVPYGLSNYRX₆SX₇RGKR (SEQ ID No. 20), and wherein X₁, X₂, X₃, X₄, X₅, X₆, and X₇ are independently selected from any amino acid. In some embodiments, the first polypeptide portion comprises the amino acid sequence X₁TX₂FTYKDKEX₃VYYX₄HLDIIX₅INTPEQTVPYGLSNYRX₆SX₇RGKR (SEQ ID No. 20), and wherein X₁, X₂, X₃, X₄, X₅, X₆, and X₇ are independently selected from any amino acid. In some embodiments, the polypeptide comprises the amino acid sequence X₁TX₂FTYKDKEX₃VYYX₄HLDIIX₅INTPEQTVPYGLSNYRX₆S (SEQ ID No. 27), and wherein X₁, X₂, X₃, X₄, X₅, and X₆ are independently selected from any amino acid. In some embodiments, the first polypeptide portion comprises the amino acid sequence X₁TX₂FTYKDKEX₃VYYX₄HLDIIX₅INTPEQTVPYGLSNYRX₆S (SEQ ID No. 27), and wherein X₁, X₂, X₃, X₄, X₅, and X₆ are independently selected from any amino acid.

In some embodiments, X₄ is C, S, or A. In some embodiments, X₅ is W or A. In some embodiments, X₁ is S or C. In some embodiments, X₂ is S or C. In some embodiments, X₃ is S or C. In some embodiments, X₆ is G or E. In some embodiments, X₇ is F or L.

In some embodiments, the metabolic disease or disorder is obesity. In some embodiments, the metabolic disease or disorder is type I diabetes or type II diabetes. In some embodiments, the metabolic disease or disorder is insulin resistance. In some embodiments, the metabolic disease or disorder is a lipid metabolic disorder. In some embodiments, the metabolic disease or disorder is hyperlipidemia. In some embodiments, the metabolic disease or disorder is hypercholesterolemia. In some embodiments, the metabolic disease or disorder is a fatty acid metabolism disorder.

In certain aspects, this disclosure provides a method of increasing core body temperature, comprising administering to a subject in need thereof an effective amount of the EDN3-like polypeptide. In certain aspects, this disclosure provides a method of increasing core body temperature, comprising administering to a subject in need thereof an effective amount of a composition comprising an EDN3-like polypeptide.

In certain aspects, this disclosure provides a method of increasing core body temperature, comprising administering to a subject in need thereof an effective amount of an isolated polypeptide of less than or equal to 60 amino acid residues in length, wherein the polypeptide comprises the amino acid sequence YYCHLDIIWINTPEQ (SEQ ID No. 7) or an amino acid sequence having 1, 2, or 3 substitutions relative to SEQ ID No. 7, and wherein the polypeptide does not include gastric inhibitory peptide.

In certain aspects, this disclosure provides a method of increasing core body temperature, comprising administering to a subject in need thereof an effective amount of a polypeptide comprising (a) a first polypeptide portion comprising the amino acid sequence YYCHLDIIWINTPEQ (SEQ ID No. 7) or an amino acid sequence having 1, 2, or 3 substitutions relative to SEQ ID No. 7, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of increasing core body temperature, comprising administering to a subject in need thereof an effective amount of an isolated polypeptide of less than or equal to 60 amino acid residues in length, wherein the polypeptide comprises the amino acid sequence YYX₄HLDIIX₅INTPEQ (SEQ ID No. 8), wherein X₄ and X₅ are independently selected from any amino acid, and wherein the polypeptide does not include gastric inhibitory peptide.

In certain aspects, this disclosure provides a method of increasing core body temperature, comprising administering to a subject in need thereof an effective amount of a polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of elevating energy expenditure, comprising administering to a subject in need thereof an effective amount of the EDN3-like polypeptide.

In certain aspects, this disclosure provides a method of elevating energy expenditure, comprising administering to a subject in need thereof an effective amount of the composition comprising an EDN3-like polypeptide.

In certain aspects, this disclosure provides a method of elevating energy expenditure, comprising administering to a subject in need thereof an effective amount of an isolated polypeptide of less than or equal to 60 amino acid residues in length, wherein the polypeptide comprises the amino acid sequence YYCHLDIIWINTPEQ (SEQ ID No. 7) or an amino acid sequence having 1, 2, or 3 substitutions relative to SEQ ID No. 7, wherein the polypeptide does not include gastric inhibitory peptide.

In certain aspects, this disclosure provides a method of elevating energy expenditure, comprising administering to a subject in need thereof an effective amount of a polypeptide comprising (a) a first polypeptide portion comprising the amino acid sequence YYCHLDIIWINTPEQ (SEQ ID No. 7) or an amino acid sequence having 1, 2, or 3 substitutions relative to SEQ ID No. 7, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of elevating energy expenditure, comprising administering to a subject in need thereof an effective amount of an isolated polypeptide of less than or equal to 60 amino acid residues in length, wherein the polypeptide comprises the amino acid sequence YYXHLDIIX₅INTPEQ (SEQ ID No. 8), wherein X₄ and X₅ are independently selected from any amino acid, wherein the polypeptide does not include ₄gastric inhibitory peptide.

In certain aspects, this disclosure provides a method of elevating energy expenditure, comprising administering to a subject in need thereof an effective amount of a polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In some embodiments, the polypeptide is capable of one or more of: inhibiting glucose production in hepatocytes, promoting GLP-1 secretion in the rat perfused colon assay, promoting GLP-1 secretion in GLUTag cells, promoting glucose uptake in skeletal muscle cells, or promoting glucose uptake in adipocytes.

In certain aspects, this disclosure provides a method of inhibiting glucose production in hepatocytes, comprising contacting hepatocytes with an effective amount of the EDN3-like polypeptide. In certain aspects, this disclosure provides a method of inhibiting glucose production in hepatocytes, comprising contacting hepatocytes with an effective amount of the composition comprising an EDN3-like polypeptide.

In certain aspects, this disclosure provides a method of inhibiting glucose production in hepatocytes, comprising contacting hepatocytes with an effective amount of an isolated polypeptide of less than or equal to 60 amino acid residues in length, wherein the polypeptide comprises the amino acid sequence YYCHLDIIWINTPEQ (SEQ ID No. 7) or an amino acid sequence having 1, 2, or 3 substitutions relative to SEQ ID No. 7, wherein the polypeptide does not include gastric inhibitory peptide.

In certain aspects, this disclosure provides a method of inhibiting glucose production in hepatocytes, comprising contacting hepatocytes with an effective amount of a polypeptide comprising (a) a first polypeptide portion comprising the amino acid sequence YYCHLDIIWINTPEQ (SEQ ID No. 7) or an amino acid sequence having 1, 2, or 3 substitutions relative to SEQ ID No. 7, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of inhibiting glucose production in hepatocytes, comprising contacting hepatocytes with an effective amount of an isolated polypeptide of less than or equal to 60 amino acid residues in length, wherein the polypeptide comprises the amino acid sequence YYX₄HLDIIX₅INTPEQ (SEQ ID No. 8), wherein X₄ and X₅ are independently selected from any amino acid, wherein the polypeptide does not include gastric inhibitory peptide.

In certain aspects, this disclosure provides a method of inhibiting glucose production in hepatocytes, comprising contacting hepatocytes with an effective amount of a polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of promoting glucagon-like peptide-1 (GLP-1) secretion, comprising contacting enteric cells with an effective amount of the EDN3-like polypeptide. In certain aspects, this disclosure provides a method of promoting GLP-1 secretion, comprising contacting enteric cells with an effective amount of the composition comprising an EDN3-like polypeptide.

In certain aspects, this disclosure provides a method of promoting GLP-1 secretion, comprising contacting enteric cells with an effective amount of an isolated polypeptide of less than or equal to 60 amino acid residues in length, wherein the polypeptide comprises the amino acid sequence YYCHLDIIWINTPEQ (SEQ ID No. 7) or an amino acid sequence having 1, 2, or 3 substitutions relative to SEQ ID No. 7, wherein the polypeptide does not include gastric inhibitory peptide.

In certain aspects, this disclosure provides a method of promoting GLP-1 secretion, comprising contacting enteric cells with an effective amount of a polypeptide comprising (a) a first polypeptide portion comprising the amino acid sequence YYCHLDIIWINTPEQ (SEQ ID No. 7) or an amino acid sequence having 1, 2, or 3 substitutions relative to SEQ ID No. 7, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of promoting GLP-1 secretion, comprising contacting enteric cells with an effective amount of an isolated polypeptide of less than or equal to 60 amino acid residues in length, wherein the polypeptide comprises the amino acid sequence YYX₄HLDIIX₅INTPEQ (SEQ ID No. 8), wherein X₄ and X₅ are independently selected from any amino acid, wherein the polypeptide does not include gastric inhibitory peptide.

In certain aspects, this disclosure provides a method of promoting GLP-1 secretion, comprising contacting enteric cells with an effective amount of a polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In some embodiments, the enteric cells are colon cells or GLUTag cells.

In certain aspects, this disclosure provides a method of promoting glucose uptake in skeletal muscle cells, comprising contacting skeletal muscle cells with an effective amount of the EDN3-like polypeptide. In certain aspects, this disclosure provides a method of promoting glucose uptake in skeletal muscle cells, comprising contacting skeletal muscle cells with an effective amount of the composition comprising an EDN3-like polypeptide.

In certain aspects, this disclosure provides a method of promoting glucose uptake in skeletal muscle cells, comprising contacting skeletal muscle cells with an effective amount of an isolated polypeptide of less than or equal to 60 amino acid residues in length, wherein the polypeptide comprises the amino acid sequence YYCHLDIIWINTPEQ (SEQ ID No. 7) or an amino acid sequence having 1, 2, or 3 substitutions relative to SEQ ID No. 7, and wherein the polypeptide does not include gastric inhibitory peptide.

In certain aspects, this disclosure provides a method of promoting glucose uptake in skeletal muscle cells, comprising contacting skeletal muscle cells with an effective amount of an isolated polypeptide of less than or equal to 60 amino acid residues in length, wherein the polypeptide comprises the amino acid sequence YYX₄HLDIIX₅INTPEQ (SEQ ID No. 8), wherein X₄ and X₅ are independently selected from any amino acid, wherein the polypeptide does not include gastric inhibitory peptide.

In certain aspects, this disclosure provides a method of promoting glucose uptake in skeletal muscle cells, comprising contacting skeletal muscle cells with an effective amount of a polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of promoting glucose uptake in adipocytes, comprising contacting adipocytes with an effective amount of the EDN3-like polypeptide. In certain aspects, this disclosure provides a method of promoting glucose uptake in adipocytes, comprising contacting adipocytes with an effective amount of the composition comprising an EDN3-like polypeptide.

In certain aspects, this disclosure provides a method of promoting glucose uptake in adipocytes, comprising contacting adipocytes with an effective amount of an isolated polypeptide of less than or equal to 60 amino acid residues in length, wherein the polypeptide comprises the amino acid sequence YYCHLDIIWINTPEQ (SEQ ID No. 7) or an amino acid sequence having 1, 2, or 3 substitutions relative to SEQ ID No. 7, wherein the polypeptide does not include gastric inhibitory peptide.

In certain aspects, this disclosure provides a method of promoting glucose uptake in adipocytes, comprising contacting adipocytes with an effective amount of a polypeptide comprising (a) a first polypeptide portion comprising the amino acid sequence YYCHLDIIWINTPEQ (SEQ ID No. 7) or an amino acid sequence having 1, 2, or 3 substitutions relative to SEQ ID No. 7, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In certain aspects, this disclosure provides a method of promoting glucose uptake in adipocytes, comprising contacting adipocytes with an effective amount of an isolated polypeptide of less than or equal to 60 amino acid residues in length, wherein the polypeptide comprises the amino acid sequence YYX₄HLDIIX₅INTPEQ (SEQ ID No. 8), wherein X₄ and X₅ are independently selected from any amino acid, wherein the polypeptide does not include gastric inhibitory peptide.

In certain aspects, this disclosure provides a method of promoting glucose uptake in adipocytes, comprising contacting adipocytes with an effective amount of an isolated polypeptide comprising (a) a first polypeptide portion comprising the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second polypeptide portion, which second polypeptide portion is heterologous to said first polypeptide portion, and wherein the polypeptide does not include gastric inhibitory peptide.

In some embodiments, the adipocytes are derived from human adipose stem cells (hASC) or human mesenchymal stem cells (hMSC).

In some embodiments, the method is performed in vitro. In some embodiments, the method is performed in vivo. In some embodiments, the polypeptide does not bind to endothelin receptor A (ET_{A}). In some embodiments, the polypeptide does not bind to endothelin receptor B (ET_{B}). In some embodiments, the polypeptide is a peptidomimetic.

In some embodiments, the polypeptide comprises the amino acid sequence X₁TX₂FTYKDKEX₃VYYX₄HLDIIX₅INTPEQT (SEQ ID No. 33), wherein: X₁ and X₄ are C and X₂ and X₃ are independently selected from any amino acid, or X₂ and X₃ are C and X₁ and X₄ are independently selected from any amino acid, and X₅ is any amino acid.

In some embodiments, the polypeptide comprises the amino acid sequence X₁TX₂FTYKDKEX₃VYYX₄HLDIIX₅INTPEQTVPYGLSNYRX₆SX₇R (SEQ ID No. 14), wherein: X₁ and X₄ are C and X₂ and X₃ are A, or X₂ and X₃ are C and X₁ and X₄ are A, X₅ is W, F, or Y, X₆ is E or G, and X₇ is L or F.

In some embodiments, the first polypeptide portion comprises the amino acid sequence X₁TX₂FTYKDKEX₃VYYX₄HLDIIX₅INTPEQT (SEQ ID No. 33), wherein: X₁ and X₄ are C and X₂ and X₃ are independently selected from any amino acid, or X₂ and X₃ are C and X₁ and X₄ are independently selected from any amino acid, and X₅ is any amino acid.

In some embodiments, the polypeptide comprises the amino acid sequence X₁TX₂FTYKDKEX₃VYYX₄HLDIIX₅INTPEQTVPYGLSNYRX₆SX₇R (SEQ ID No. 14), wherein: X₁ and X₄ are C and X₂ and X₃ are A, or X₂ and X₃ are C and X₁ and X₄ are A, X₅ is W, F, or Y, X₆ is E or G, and X₇ is L or F.

In certain aspects, this disclosure provides a method of identifying warm-sensitive neurons, comprising contacting a brain tissue sample with an antibody that binds specifically to a polypeptide comprising the amino acid sequence CTCFTYKDKECVYYCHLDIIWINTPEQTVPYGLSNYRGSFRGKR (SEQ ID No. 19) or CTCFTYKDKECVYYCHLDIIWINTPEQTVPYGLSNYRESLRGKR (SEQ ID No. 21).

In certain aspects, this disclosure provides a method of identifying warm-sensitive neurons, comprising contacting a brain tissue sample with a nucleic acid probe or primer that hybridizes under stringent conditions to a nucleic acid encoding a polypeptide comprising the amino acid sequence YYCHLDIIWINTPEQ (SEQ ID No. 7).

In certain aspects, this disclosure provides a method of identifying an EDN3-like receptor, comprising: (a) contacting a test cell with an EDN3-like polypeptide and a receptor inhibitor, (b) contacting a control cell with an EDN3-like polypeptide, and (c) determining the EDN3-like response of the test cell and control cell, wherein a greater EDN3-like response of the control cell compared to the test cell indicates that the receptor inhibited by the receptor inhibitor is an EDN3-like receptor.

In certain aspects, this disclosure provides a method of identifying an EDN3-like receptor, comprising: (a) contacting a test cell with an EDN3-like polypeptide, wherein the test cell comprises a mutation that reduces the activity of a receptor, (b) contacting a control cell with an EDN3-like polypeptide, wherein the control cell comprises wild-type activity of the receptor, and (c) determining the EDN3-like response of the test cell and control cell, wherein a greater EDN3-like response of the control cell compared to the test cell indicates that the receptor inhibited by the receptor antagonist is an EDN3-like receptor.

In certain aspects, this disclosure provides a method of identifying a putative EDN3-like receptor, comprising contacting a cell lysate with an EDN3-like polypeptide and isolating a protein that binds the EDN3-like polypeptide, wherein the protein that binds the EDN3-like polypeptide is a putative EDN3-like receptor.

In certain aspects, this disclosure provides a method of identifying an EDN3-like receptor, comprising contacting an EDN3-like polypeptide with a candidate receptor and determining whether the EDN3-like polypeptide binds the candidate receptor, where binding indicates that the candidate receptor is an EDN3-like receptor.

In certain aspects, the disclosure provides a method of generating an image of a subject material comprising: (a) providing a subject material comprising a plurality of cells wherein a subset of cells comprise a detectable amount of a detectably labeled EDN3-like compound; and (b) imaging the cells. In certain aspects, the disclosure provides a method of generating an image of a subject material comprising: (a) providing a subject material comprising a plurality of cells wherein a subset of cells comprise a detectable amount of a detectably labeled EDN3-like polypeptide; and (b) imaging the cells. The detectable label may be, for example, a radiolabel, an MRI-detectable label, or a fluorescent label. The cells may be imaged by, for example, detecting radioactivity (e.g., by gamma camera), detecting fluorescence (e.g., with a CCD camera), or by MRI. Note that this method of imaging (or generating an image) may be used, in certain embodiments, to identify cells and tissues that express a receptor for the EDN3-like polypeptide.

In certain embodiments of any of the foregoing, the EDN3-like polypeptide for use in any of the disclosed methods comprises or consists of the amino acid sequence represented in any of SEQ ID NOs 1-33. In certain embodiments, the EDN3-like polypeptide is a polypeptide of less than or equal to 60 amino acids and comprises an amino acid sequence represented in any of SEQ ID NOs 1-33. In other embodiments, the EDN3-like polypeptide is a polypeptide of less than or equal to 50 amino acids, or of about 44 amino acids, or of about 44-60 amino acids. Such EDN3-like polypeptide may be provided alone or as a portion of a fusion protein fused to a second heterologous polypeptide portion (e.g., an Fc domain, an epitope tag, a linker, etc.). It is specifically contemplated that any such EDN3-like polypeptides, as well as any other EDN3-like polypeptides and variants may be used in any of the methods described herein and/or may be provided as isolated polypeptides or as fusion proteins.

The disclosure contemplates all combinations of any one or more of the foregoing aspects and/or embodiments, as well as combinations with any one or more of the embodiments set forth in the detailed description and examples.

### BRIEF DESCRIPTION OF THE DRAWINGS

FIG. 1. Primary sequence of long endothelin-3 from the mouse hypothalamus. Using ProP 1.0 analysis to identify arginine and lysine cleavage sites, 2 propeptide cleavage sites of endothelin-3 were identified, resulting in a 44 amino acid peptide. The signal sequence is notated by S. The predicted peptide from this analysis, along with corresponding cleavage sites, is annotated by P.

FIG. 2. EDN3 97-140 does not evoke a vasoconstriction response in rat aortic vessels. EDN3 97-140 at 3 pM-10 μM failed to elicit a contractile response in isolated rat aortic rings. In contrast, endothelin-1 (EC₅₀ 2.7±1 nM) and endothelin-3 (EC₅₀ 102±63 nM) potently evoked concentration-dependent contractions in this model (n=4-6; p>0.05).

FIG. 3Ai-3C. EDN3 97-140 reduces respiratory exchange ratio (RER) and increases core body temperature (CBT). Mice (6 per group) were treated with 2.5 nmol EDN3 97-140 or vehicle by direct injection into the preoptic area (POA). Compared to vehicle treated mice, the area under the curve (AUC) of RER decreased by 1.46±0.25 (p=0.002; Ai & B) and the AUC of CBT increased by 14.97±2.85 (p=0.002; Aii & C) after EDN3 97-140 injection. Results are mean AUC±SEM. In contrast to sustained effects on RER and CBT, a transient spike of increased locomotor activity was noted upon EDN3 97-140 administration (Aiii).

FIG. 4Ai-Bii. EDN3 97-140 improves glucose tolerance in ob/ob and DIO mice. Following a single bolus of EDN3 97-140 (i.p.), blood glucose concentrations were measured after an overnight fast and following a 0.6 mg/kg or 2 g/kg glucose challenge (i.p.) in ob/ob or DIO mice, respectively. In ob/ob mice there was a 35.1±13% reduction in glucose at 1 mg/kg and 31.3±6.9% reduction at 10 mg/kg (Ai). Additionally, while a trend towards an increase in insulin secretion at 30 minutes at 10 mg/kg was observed, this was not statistically significant (p>0.05; Aii). Likewise, in DIO mice, 0.1 mg/kg and 1 mg/kg decreased the glucose excursion by 19±7.2% and 20.7±8.7%, respectively (Bi) with no effect on insulin (p>0.05; Bii). Data are represented as mean±SEM (n=8 per group) and * denotes p<0.05.

FIG. 5A-D. EDN3 97-140 stimulates GLP1 secretion from GLUTag cells in vitro and rat colon ex vivo. EDN3 97-140 stimulates GLP-1 secretion from GLUTag cells with an EC₅₀ of 369 nM±68 nM across 3 independent experiments (Ai). GLUTag cells were pre-treated overnight with vehicle or 0.2 μg/mL cholera toxin (CTX) and then treated with EDN3 97-140 (1 μM) or vehicle control. The non subtype-selective ET_{A}/ET_{B} antagonist bosentan (1 μM) did not inhibit the EDN3 97-140 stimulated GLP-1 secretion from GLUTag cells (FIG. 5B). EDN3 97-140 (1 μM) stimulated GLP-1 secretion was 327±59% of vehicle and overnight pre-treatment with CTX reduced EDN3 97-140 stimulated GLP-1 secretion to 139±19% of vehicle (p=0.012; FIG. 5C). EDN3 97-140 (200 nM) elicited a 56±18% increase in GLP secretion from the rat perfused colon (p=0.0438). The positive control forskolin (1 μM) stimulated GLP-1 release by 1717±350% (p=0.0398; FIG. 5D).

FIG. 6. EDN3 97-140 suppresses gluconeogenesis. Overnight treatment of H4IIE rat hepatocytes with 100 nM EDN3 97-140 significantly reduced basal glucose production by 19.5±7.6% (p=0.01) versus vehicle. Data is graphed as mean of 3 independent experiments±SEM.

FIG. 7. This figure shows the sequence of preproendothelin-3 from Mus musculus (SEQ ID NO: 45).

### DETAILED DESCRIPTION

1. EDN3-Like Polypeptide Compositions of Matter

This disclosure is based in part on the identification of a short polypeptide that is referred to herein as EDN3 97-140, and the discovery that this polypeptide affects glucose metabolism and body temperature regulation. EDN3 97-140 is a short polypeptide that may be produced endogenously by cleavage of preproendothelin-3. Proteolytic cleavage of the preproendothelin-3 precursor also produces a different peptide known as Endothelin-3. EDN3 97-140 and Endothelin-3 share a common N-terminus, but Endothelin-3 is only 21 amino acids in length while EDN3 97-140 is 44 amino acids long. In addition to these structural differences, EDN3 97-140 and Endothelin-3 have distinct functional activities. For example, EDN3 97-140 promotes GLP-1 release from enteroendocrine cells, and Endothelin-3 does not (Example 6). Conversely, Endothelin-3 promotes aortic vasoconstriction, and EDN3 97-140 does not (Example 2). Moreover, the activities of EDN3 97-140 and endothelin-3 are mediated by different receptors.

The present disclosure provides EDN3-like polypeptides. This disclosure describes peptide therapeutics that include a polypeptide comprising a sequence set forth in any one of SEQ ID NOs: 1-33 or a variant thereof, and these sequences are listed in Table 1 below. In some cases the polypeptide is a short peptide fragment, and in other cases the polypeptide is provided as a fusion with another polypeptide portion. Throughout this disclosure, the term EDN3-like polypeptides shall be used interchangeably to refer to peptides and fusions having the desired activity. Of the specific sequences in Table 1, SEQ ID NOs: 19 and 21 may correspond to endogenous peptides (see Example 1) in humans and mice, respectively. SEQ ID NOs: 19 and 21 have been named hEDN3 97-140 and EDN3 97-140 respectively, because they are predicted to be produced by cleavage of pre-proendothelin 3 (Example 1). These are two examples of EDN3-like polypeptides.

hEDN3 97-140 and EDN3 97-140, as well as the other peptides of Table 1 and fusion proteins and variants thereof, are collectively referred to herein as EDN3-like polypeptides. However, EDN3-like polypeptides possess activities that endothelin 3 does not. For instance, as shown in Example 5, EDN3 97-140 stimulates GLP-1 secretion in GLUTag cells, and endothelin-3 does not. Thus, EDN3-like polypeptides have one or more activities selected from: promoting GLP-1 release, inhibiting hepatic gluconeogenesis, increasing core body temperature, and increasing respiratory exchange ratio. The term “EDN3-like polypeptides” excludes Endothelin-3 (SEQ ID NO: 40) and preproendothelin-3. Note that the terms polypeptide and peptide are used interchangeably throughout.

The activity of EDN3-like polypeptides can be measured by one or more of the assays disclosed herein. For instance, Example 5 shows that several EDN3-like polypeptides have GLP-1 release activity. Moreover, Example 8 discloses a method for assaying the ability of EDN3-like polypeptides to inhibit gluconeogenesis in hepatocytes. In addition, Example 3 discloses a method for assaying hyperthermia and respiratory exchange ratio in mice. Some EDN3-like polypeptides, like EDN3 97-140, have activity in all of the assays. However, this application contemplates that some EDN3-like polypeptides suitable for use may have activity in a subset of the assays (e.g., 1, 2, 3, etc.). Following the assays disclosed herein, one can determine which activities a given EDN3-like polypeptide possesses, and thus readily make, test and select those polypeptides suitable for use in the claimed methods.

EDN3-like polypeptides include variants. At some positions of the EDN3-like amino acid sequences (e.g., residues X₆ and X₇ of SEQ ID NO: 2) variants have been and can be designed taking into account interspecies sequence comparisons. In some instances (e.g., residue X₅ of SEQ ID NO: 2) variants have been and can be designed based on experimental data. In certain instances, such as with SEQ ID NOs: 2 and 3, a core region was identified based on the activity of certain truncation mutants. The experiments that were used to identify variable residues and core regions are described in more detail in Example 6.

TABLE 1
Selected EDN3-like polypeptides

| | | SEQ |
|---|---|---|
| | | ID |
| Name | Amino acid sequence | NO |
| EDN3 97-136 | CTCFTYKDKECVYYCHLDIIWINTPEQTVPY | 1 |
| | GLSNYRESL | |
| EDN3 97-136 | X₁TX₂FTYKDKEX₃VYYX₄HLDIIX₅INTPE | 2 |
| consensus | QTVPYGLSNYRX₆SX₇ | |
| EDN3 109-123 | YYSHLDIIWINTPEQ | 3 |
| C111S | | |
| EDN3 109-123 | YYAHLDIIAINTPEQ | 4 |
| C111A W117A | | |
| EDN3 109-137 | YYSHLDIIWINTPEQTVPYGLSNYRX₆SX₇R | 5 |
| C111S | | |
| consensus | | |
| EDN3 109-137 | YYAHLDIIAINTPEQTVPYGLSNYRX₆SX₇R | 6 |
| C111A W117A | | |
| consensus | | |
| EDN3 109-123 | YYCHLDIIWINTPEQ | 7 |
| EDN3 109-123 | YYX₄HLDIIX₅INTPEQ | 8 |
| consensus | | |
| EDN3 97-123 | CTCFTYKDKECVYYCHLDIIWINTPEQ | 9 |
| EDN3 97-123 | X₁TX₂FTYKDKEX₃VYYX₄HLDIIX₅INTPE | 10 |
| consensus | Q | |
| EDN3 97-127 | CTCFTYKDKECVYYCHLDIIWINTPEQTVPY | 11 |
| EDN3 97-127 | X₁TX₂FTYKDKEX₃VYYX₄HLDIIX₅INTPE | 12 |
| consensus | QTVPY | |
| hEDN3 97-137 | CTCFTYKDKECVYYCHLDIIWINTPEQTVPY | 13 |
| | GLSNYRGSFR | |
| EDN3 97-137 | X₁TX₂FTYKDKEX₃VYYX₄HLDIIX₅INTPE | 14 |
| consensus | QTVPYGLSNYRX₆SX₇R | |
| EDN3 109-137 | YYCHLDIIWINTPEQTVPYGLSNYRGSFR | 15 |
| EDN3 109-137 | YYX₄HLDIIX₅INTPEQTVPYGLSNYRX₆S | 16 |
| consensus | X₇R | |
| EDN3 109-140 | YYCHLDIIWINTPEQTVPYGLSNYRGSFRGK | 17 |
| | R | |
| EDN3 109-140 | YYX₄HLDIIX₅INTPEQTVPYGLSNYRX₆S | 18 |
| consensus | X₇RGKR | |
| hEDN3 97-140 | CTCFTYKDKECVYYCHLDIIWINTPEQTVPY | 19 |
| | GLSNYRGSFRGKR | |
| EDN3 97-140 | X₁TX₂FTYKDKEX₃VYYX₄HLDIIX₅INTPE | 20 |
| consensus | QTVPYGLSNYRX₆SX₇RGKR | |
| EDN3 97-140 | CTCFTYKDKECVYYCHLDIIWINTPEQTVPY | 21 |
| | GLSNYRESLRGKR | |
| EDN3 109-140 | YYSHLDIIWINTPEQTVPYGLSNYRX₆SX₇R | 22 |
| C111S | GKR | |
| consensus | | |
| EDN3 109-140 | YYAHLDIIAINTPEQTVPYGLSNYRX₆SX₇R | 23 |
| C111A W117A | GKR | |
| consensus | | |
| EDN3 102-123 | YKDKECVYYCHLDIIWINTPEQ | 24 |
| EDN3 102-123 | YKDKEX₃VYYX₄HLDIIX₅INTPEQ | 25 |
| Consensus | | |
| EDN3 97-135 | CTCFTYKDKECVYYCHLDIIWINTPEQTVPY | 26 |
| | GLSNYRES | |
| EDN3 97-135 | X₁TX₂FTYKDKEX₃VYYX₄HLDIIX₅INTPE | 27 |
| consensus | QTVPYGLSNYRX₆S | |
| hEDN3 97-135 | CTCFTYKDKECVYYCHLDIIWINTPEQTVPY | 28 |
| | GLSNYRGS | |
| EDN3 97-137 | CTCFTYKDKECVYYCHLDIIWINTPEQTVPY | 29 |
| | GLSNYRESLR | |
| EDN3 97-140 | CTCFTYKDKECVYYCHLDIIFINTPEQTVPY | 30 |
| W117F | GLSNYRESLRGKR | |
| EDN3 97-140 | ATCFTYKDKECVYYAHLDIIWINTPEQTVPY | 31 |
| C97A, C111A | GLSNYRESLRGKR | |
| EDN3 97-140 | CTAFTYKDKEAVYYCHLDIIWINTPEQTVPY | 32 |
| C99A, C107A | GLSNYRESLRGKR | |
| EDN3 97-124 | X₁TX₂FTYKDKEX₃VYYX₄HLDIIX₅INTPE | 33 |
| consensus | QT | |

Peptides comprising regions homologous to the peptides of Table 1 may also be used in the methods and compositions herein (e.g., variants). For instance, because the human and mouse EDN3-like sequences are disclosed herein, one of skill in the art could readily substitute an amino acid sequence from one organism, or a portion thereof, with the homologous amino acid sequence from the other organism. In certain aspects, this application provides polypeptides with a region having 1, 2, or 3 substitutions relative to a peptide of Table 1, or an active fragment thereof. For EDN3-like polypeptides comprising a substitution, it is understood that the substitution can be (in some embodiments) a conservative substitution of the corresponding residue in SEQ ID NO: 19 or 21. Certain of the peptides listed in Table 1 contain variable residues that are represented with an X. Sometimes, the identity of the variable residue is the amino acid that naturally occurs at that position in the human or mouse sequence. In certain aspects, this application provides polypeptides having a region with at least 80%, 85%, 90%, 95%, 97%, 98%, or 99% identity to a peptide of Table 1.

For instance, in some embodiments, the application provides EDN3-like polypeptides comprising or consisting of a sequence set forth in any one of SEQ ID NOs: 1-33 or a variant thereof. Also contemplated, are EDN3-like polypeptides having a length of less than or equal to 60 amino acid residues comprising a sequence set forth in any of SEQ ID NOs 1-33 (e.g., the polypeptide comprises the sequence, but the total length of the polypeptide is less than or equal to 60 amino acid residues. Where the sequence includes variable residues, each variable amino acid is, in some embodiments, independently selected from any amino acid. In certain embodiments, one or more variable amino acids is selected from the corresponding amino acid in one of SEQ ID NOs: 19-21 or a conservative substitution thereof. In certain embodiments, the EDN3-like polypeptide has 1, 2, or 3 substitutions relative to the amino acid sequence set forth in one of SEQ ID NOs: 1-33. In some embodiments, a variant of one of SEQ ID NOs: 1-33 is a polypeptide lacking 1 or 2 amino acids from one or both termini. In some embodiments, the EDN3-like polypeptide comprises a first portion and a second portion, the first portion consisting of an EDN3-like polypeptide as described herein (e.g., one of SEQ ID NOs: 1-33 or a variant thereof). The second portion may be heterologous to the first portion or may be a detectable label.

It is understood that this disclosure provides all combinations and sub-combinations of any one or more of the aspects and embodiments described herein. For instance, with respect to compositions of matter, this disclosure provides peptides consisting of or comprising each of SEQ ID NOs: 1-33 and variants thereof in various forms: alone, in the context of a fusion protein, as a truncation variant, as a substitution variant, as a variant with an internal deletion, and in suitable combinations of these forms. In addition, this disclosure explicitly contemplates the use of any of the EDN3-like peptides described herein (for instance a polypeptide comprising or consisting of any of SEQ ID NOs: 1-33 or a variant thereof) for use in any of the methods disclosed herein. Several methods of treatment are described in more detail in Section 5 below. Merely as examples, EDN3-like polypeptides may be used to treat a metabolic disease or disorder, type I diabetes, type II diabetes, insulin resistance, a lipid metabolic disorder, hyperlipidemia, hypercholesterolemia, or a fatty acid metabolism disorder.

In some aspects, the EDN3-like polypeptide has one substitution relative to SEQ ID No. 21 or a fragment thereof.

In some embodiments, this application provides truncation variants that are close in size to an EDN3-like polypeptide. For example, such variants may lack at most one, two, three, four, or five amino acids from one or both termini. As specific examples of truncation mutants, the N-terminal most amino acid may be T2, C3, T4, C5, F6, T7, Y8, K9, D10, K11, E12, or C13. As further examples, the C-terminal most amino acid may be R41, G42, or K43. While the numbering system in the previous two sentences is derived from hEDN3 97-140 (SEQ ID NO: 19), one of skill in the art will easily be able to identify the corresponding amino acid on any other EDN3-like polypeptide. Internal deletions, e.g., of one, two, three, four, or five amino acids are also contemplated. In some embodiments, the EDN3-like peptide is 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 amino acids in length. In other embodiments, the EDN3-like polypeptide is a fusion protein in which the first polypeptide portion is a EDN3-like sequence of 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 amino acids.

In certain embodiments, the EDN3-like polypeptide is between 20 and 60 amino acids in length. For instance, an EDN3-like polypeptide may be 30-60, 30-50, 30-40, 40-60, 40-50, or 50-60 amino acids long. An EDN3-like polypeptide may also be 20-25, 25-30, 30-35, 35-40, 40-45, 45-50, 50-55, or 55-60 amino acids in length. An EDN3-like polypeptide may also be 20-22, 22-24, 24-26, 26-28, 28-30, 30-32, 32-34, 34-36, 36-38, 38-40, 40-42, 42-44, 44-46, 46-48, 48-50, 50-52, 52-54, 54-56, 56-58, or 58-60 amino acids in length. In some embodiments, the first amino acid portion of an EDN3-like polypeptide is 20-25, 25-30, 30-35, 35-40, 40-45, 45-50, 50-55, 55-60, 20-22, 22-24, 24-26, 26-28, 28-30, 30-32, 32-34, 34-36, 36-38, 38-40, 40-42, 42-44, 44-46, 46-48, 48-50, 50-52, 52-54, 54-56, 56-58, or 58-60 amino acids in length.

In certain embodiments, EDN3-like polypeptides include X₁ through X₇ where each are independently selected from any amino acid. However, in some embodiments, one or more of X₁ through X₇ is a conservative substitution relative to the corresponding amino acid in the mouse sequence EDN3 97-140 (SEQ ID NO: 21) or the human sequence hEDN3 97-140 (SEQ ID NO: 19). Likewise, in some embodiments an EDN3-like polypeptide is defined as having one or more substitutions relative to a given sequence (such as SEQ ID No. 1). In certain embodiments, the polypeptides comprise 1, 2, 3, 4, or 5 substitutions. Such substitutions may be independently selected as a conservative or non-conservative substitution. In certain embodiments, all of the substitutions (e.g., 1, 2, 3, 4, 5, etc.) are conservative substitutions.

A conservative substitution is a substitution with an amino acid of similar charge, hydrophobicity, or aromatic character. For instance, substitutions among W, F, and Y are conservative because all three have aromatic rings. As another example, a substitution between D and E is conservative because both are negatively charged. Similarly, substitutions among K, R, and H are conservative because these residues are positively charged. In addition, substitutions among S, T, C, Y, N, and Q are conservative because these residues are hydrophilic. Moreover, substitutions among G, A, V, L, I, P, M, F, and W are conservative because these residues are nonpolar.

In some embodiments, the EDN3-like polypeptide is less than or equal to 60 amino acid residues in length, and the peptide comprises the amino acid sequence YYX₄HLDIIX₅INTPEQ (SEQ ID No. 8), wherein X₄ and X₅ are independently selected from any amino acid. In certain embodiments, the EDN3-like polypeptide is a fusion protein that comprises the amino acid sequence YYX₄HLDIIX₅INTPEQ (SEQ ID No. 8), wherein X₄ and X₅ are independently selected from any amino acid, and which first polypeptide portion is less than or equal to 60 amino acid residues in length, and (b) a second portion, which second portion is a polypeptide portion heterologous to said first polypeptide portion or is a detectable label. In some embodiments, the polypeptide does not include gastric inhibitory peptide.

In some embodiments, one or both of X₄ and X₅ is a conservative substitution relative to the corresponding position in the putative endogenous human and mouse sequences (SEQ ID No. 19 and SEQ ID No. 21, respectively). For instance, X₄ may be C or a conservative substitution of C, and X₅ may be W or a conservative substitution of W.

In certain aspects, the EDN3-like polypeptide comprises the amino acid sequence X₁TX₂FTYKDKEX₃VYYX₄HLDIIX₅INTPEQT (SEQ ID No. 33), wherein: X₁ and X₄ are C and X₂ and X₃ are independently selected from any amino acid, or X₂ and X₃ are C and X₁ and X₄ are independently selected from any amino acid; and X₅ is any amino acid. For EDN3-like polypeptides comprising a variable residue, it is understood that the variable residue can be selected (in some embodiments) from the residue at the corresponding position in SEQ ID No. 19 or 21 or a conservative substitution thereof.

In certain aspects, all four of X₁, X₂, X₃, and X₄ are C. In other aspects, two of X₁, X₂, X₃, and X₄ are C and the other two residues are independently selected from any amino acid; for instance, both can be A. For instance, in some embodiments, X₁ and X₄ are both C, and X₂ and X₃ are independently selected from any amino acid; for instance, both can be A. In certain embodiments, X₂ and X₃ are both C, and X₁ and X₄ are independently selected from any amino acid; for instance, both can be A. In certain embodiments, one or more of X₁, X₂, X₃, and X₄ is C. In certain embodiments, X₁ is absent. The presence of two cysteines allows formation of disulfide bonds between cysteine residues.

In some embodiments, the polypeptide is a fusion protein wherein the first amino acid portion comprises an EDN3-like sequence and the second polypeptide portion is C-terminal to the first polypeptide portion. In some such aspects, the second polypeptide is 1, 2, 3, 4, 5, 6, 7, 8, 9, 10, 11, 12, 13, 14, 15, or 16 amino acids in length.

In some embodiments, X₅ is W, F, or Y. In certain embodiments, X₅ is W or F. In some embodiments, X₅ is W.

In some embodiments, X₆ is G or E. In certain embodiments, X₇ is F or L. In certain embodiments, X₆ is G and X₇ is F, so that these positions mirror the human sequence. In other embodiments, X₆ is E and X₇ is L, so that these positions mirror the mouse sequence.

In some embodiments the EDN3-like polypeptide consists of: (i) the amino acid sequence X₁TX₂FTYKDKEX₃VYYX₄HLDIIX₅INTPEQTVPYGLSNYRX₆SX₇ (SEQ ID No. 2) or (ii) a fragment of (i) beginning at position 1 and ending at any one of positions 27 to 39 of SEQ ID No. 2; wherein X₁, X₂, X₃, X₄, X₅, X₆, and X₇ are independently selected from any amino acid.

In some embodiments, one or more of X₁, X₂, X₃, X₄, X₅, X₆, and X₇ is a conservative substitution relative to the corresponding position in the putative endogenous human and mouse sequences (SEQ ID No. 19 and SEQ ID No. 21, respectively). For instance, X₁, X₂, X₃, X₄, may each independently be C or a conservative substitution of C, X₅ may be W or a conservative substitution of W, X₆ may be G, E, or a conservative substitution of G or E, and X₇ may be F, L, or a conservative substitution of F or L. In some embodiments, X₆ is G, A, V, L, or I. In some embodiments, X₆ is D or E. In some embodiments, X₇ is F, Y, or W. In some embodiments, X₇ is G, A, V, L, or I.

In certain embodiments, the EDN3-like polypeptide consists of CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii). Optionally, the polypeptide comprises the EDN3-like polypeptide of the previous sentence and a second, heterologous peptide portion. In certain embodiments, the residues at positions 1, 3, 11, and 15 of SEQ ID No. 1 are not substituted (e.g., all four residues are C). In certain embodiments, at least two of the residues at positions 1, 3, 11, and 15 are not substituted (e.g., are C). In some aspects, the two unsubstituted (C) residues are 1 and 15; in some aspects, the two unsubstituted (C) residues are 3 and 11. It is understood that any of the sequence embodiments disclosed herein can be combined with any of the foregoing or following compositions and methods.

The disclosure contemplates EDN3-like polypeptides, including variants of the specific examples provided herein. Such variants can be readily made and tested. Suitable amino acid substitutions include alanine, glycine, serine, threonine, leucine, and isoleucine. Other suitable substitutions include conservative substitutions. Still other substitutions include a non-conservative substitution relative to the native EDN3 97-140 sequences.

In certain embodiments, an EDN3-like polypeptide comprises only amino acids selected from the twenty canonical amino acids. In other embodiments, an EDN3-like polypeptide is a peptidomimetic. For instance, a peptidomimetic may have a wild-type peptide backbone but contain non-naturally occurring amino acids. In other instances, a peptidomimetic may have an artificial backbone. Peptidomimetics sometimes display improved stability, solubility, bioavailability, immunogenicity profile, or activity relative to the corresponding canonical polypeptide.

An EDN3-like peptidomimetic may comprise D-amino acids, a combination of D- and L-amino acids, and various amino acid analogs (e.g., β-methyl amino acids, Cα-methyl amino acids, and Nα-methyl amino acids, etc.) to confer desirable properties on peptides. Appropriate amino acid analogs include 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); HIC (histidine cyclic urea); LL-Acp (LL-3-amino-2-propenidone-6-carboxylic acid).

In certain embodiments, an EDN3-like peptidomimetic has a structural alteration to the backbone. For instance, various isosteres of amide bonds such as sulfones, trifluoroethylamines, esters, tetrazoles, or cis-amide bond isosteres may be used (Jones et al., Tetrahedron Lett. 29: 3853-3856 (1988), Black et al. “Trifluoroethylamines as amide isosteres in inhibitors of cathepsin K” Bioorganic & Medicinal Chemistry Letters, 15: 21, 1 Nov. 2005, p. 4741-4744).

An EDN3-like peptide may be prepared as a linear peptide or as a cyclic peptide. If linear, the peptide may have a free acid on the C-terminus or may be C-terminally amidated.

In certain embodiments, the EDN3-like polypeptide is linked to a detectable label such as a fluorescent label, a radiolabel, or an MRI-detectable label. Fluorescent labels include, for instance, Texas Red, phycoerythrin (PE), cytochrome c, Cy2, Cy3, Cy3.5, Cy5, Cy5.5, Cy7, fluorescent isothiocyante (FITC), tetramethylrhodamine isothiocyanate (TRITC), allophycocyanin (APC), an Alexa Fluor dye, a quantum dot dye, fluorescein, rhodamine, umbeliferone, DRAQ5, acridone, quinacridone, a lanthanide chelate, a ruthenium complexe, tartrazine, phycocyanin, or allophycocyanin. MRI-detectable labels include, for instance, a paramagnetic imaging agent, superparamagnetic iron-oxide particles, magnetite particles, a fluorocarbon imaging reagent, a Gd chelate, or a Mn chelate. Some specific MRI-detectable labels are gadopentetate dimeglumine, gadoteridol, gadoterate meglumine, mangafodipir trisodium, gadodiamide, and perfluorocarbons. The EDN3-like polypeptides may also be conjugated to a radiolabel. Examples of appropriate radiolabels include ²H, ³H, ¹¹C, ¹³C, ¹⁴C, ¹⁵N, ¹⁸O, ¹⁷O, ¹⁸F, ³⁶Cl, ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb, ^{81m}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi or ²¹³Bi. Exemplary methods for linking a given chemical moiety to a polypeptide are described herein (for instance those that can link two proteins together) and are also well know in the art.

In some embodiments, the EDN3-like polypeptide is isotopically labeled such that one or more atoms in the peptide is replaced by one or more atoms having specific atomic mass or mass numbers. Examples of isotopes that can be incorporated into proteins include isotopes of hydrogen, carbon, nitrogen, oxygen, and sulfur, such as ²H, ³H, ¹³C, ¹⁴C, ¹⁵N, ¹⁸O, ¹⁷O. Certain isotopically-labeled EDN3-like polypeptides, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled EDN3-like polypeptides can generally be prepared by substituting a readily available isotopically labeled reagent for a non-isotopically labeled reagent. Methods for doing so include some chemical linking methods that are described in the following paragraph (which can be used for, e.g., linking two proteins together) and other suitable methods are well known in the art.

In certain embodiments, an EDN3-like polypeptide is a fusion protein. A fusion protein has two or more non-overlapping polypeptide portions that are covalently joined, often with a peptide bond. The two portions may also be chemically linked using a bond other than a peptide bond. In certain embodiments, the two portions are linked or conjugated directly to each other. In other embodiments, the two portions are connected (chemically or recombinantly) via a linker. One can link two polypeptides with non-peptide bonds using a number of techniques. For instance, one can use cross-linking agents such as heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate.2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers. One useful class of heterobifunctional cross-linkers, included above, contain the primary amine reactive group, N-hydroxysuccinimide (NHS), or its water soluble analog N-hydroxysulfosuccinimide (sulfo-NHS). Another reactive group useful as part of a heterobifunctional cross-linker is a thiol reactive group. For a review of protein coupling techniques, see Means et al. (1990) Bioconjugate Chemistry. 1:2-12.

The first polypeptide portion will be a polypeptide according to Table 1 or a variant thereof (an EDN3-like polypeptide), and the second polypeptide portion will be a polypeptide that is not found contiguous to the first polypeptide portion in nature. The first polypeptide portion can be N-terminal or C-terminal to the second polypeptide portion. For instance, sometimes the second polypeptide portion represents an artificial sequence or a sequence found in a different organism. In some embodiments involving a fusion protein, the second polypeptide portion is heterologous to said first polypeptide portion. Typically, when two portions are heterologous, the two portions are not found contiguously in nature. For example, the two portions may be derived from different organisms, or one of the portions may be derived from an organism while the other portion is synthetic, or the two portions may be derived from the same organism but originate at different parts of the genome.

In some instances, the second polypeptide portion of the fusion protein comprises a tag. Well known examples of potential fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), TAP, VSV-G, V5, avidin, streptavidin, BCCP, Calmodulin, Nus, or an S tag, which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The first polypeptide portion can then be isolated from the second polypeptide portion by subsequent chromatographic separation. In certain embodiments, the second polypeptide portion may stabilize the first polypeptide portion. For example, such polypeptide may enhance the in vitro half life of the polypeptides, enhance circulatory half life of the polypeptides, or reduce proteolytic degradation of the polypeptides. The second polypeptide portion may comprise more than one epitope tag, such as 2 epitope tags, or may include 0 epitope tags.

In some embodiments, the second polypeptide portion is a fluorescent protein. Numerous fluorescent proteins are known in the art, and some examples are a green fluorescent protein (GFP), a yellow fluorescent protein (YFP), Venus, a red fluorescent protein (RFP), dsRed, mCherry, a blue fluorescent protein (BFP), and a cyan fluorescent protein (CFP).

The first polypeptide portion may be directly covalently bonded to a functional domain in the second polypeptide portion. However, in other embodiments, the second polypeptide portion may comprise a linker that links the first polypeptide portion to a functional domain in the second polypeptide portion. One exemplary linker is the (GGGGS)₃ linker (SEQ ID NO: 46). However, it is understood that other linkers may also be designed. For example, typical surface amino acids in flexible protein regions include G, N and S. Permutations of amino acid sequences containing G, N and S would be expected to satisfy the criteria (e.g., flexible with minimal hydrophobic or charged character) for a linker sequence. Other near neutral amino acids, such as T and A, can also be used in the linker sequence. In some embodiments, a linker sequence length of about 10, 15, or 20 amino acids can be used to provide a suitable separation of functional protein domains, although longer or shorter linker sequences may also be used. In certain embodiments, the second polypeptide portion may include more than one linker, such as two linkers. For embodiments in which the second polypeptide portion includes more than one linker, it is understood that the linkers are independently selected and may be the same or different.

In some embodiments, the second polypeptide portion comprises all or a portion of an Fc region of an immunoglobulin. In certain embodiments, the Fc region (or portion thereof) functions as a linker to link the first polypeptide portion to some functional domain in the second polypeptide portion. As is known, each immunoglobulin heavy chain constant region comprises four or five domains. The domains are named sequentially as follows: CH1-hinge-CH2-CH3(—CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, for instance an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In certain embodiments the immunoglobulin Fc region comprises at least an immunoglobulin hinge region a CH2 domain and a CH3 domain, and may lack the CH1 domain. In some embodiments, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of a DNA construct encoding the immunoglobulin Fc region may comprise at least a portion of a hinge domain, and may comprise at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in producing active EDN3-like fusion proteins. One example would be to introduce amino acid substitutions in the upper CH2 region to create a Fc variant with reduced affinity for Fc receptors (Cole et al. (1997) J. Immunol. 159:3613). One of ordinary skill in the art can prepare such constructs using well known molecular biology techniques.

In other embodiments, the polypeptide is free of tags such as protein purification tags, and is isolated by a method not relying on affinity for a purification tag.

In other embodiments, the second polypeptide portion is a signal sequence that promotes secretion of the fusion protein, so that it can be isolated from cell culture media. Appropriate signal sequences include the hepatitis B virus E antigen signal sequence, immunoglobulin heavy chain leader sequence, and cytokine leader sequences.

In some embodiments, the second polypeptide comprises a targeting moiety. In certain aspects, a targeting moiety may comprise an antibody, such as a monoclonal antibody, a polyclonal antibody, and a humanized antibody. Without being bound by theory, such antibody can bind to an antigen of a target tissue and thus mediate the delivery of the EDN3-like polypeptide to the target tissue (e.g., the liver or pancreas). In some embodiments, targeting moieties may comprise antibody fragments, derivatives or analogs thereof, including without limitation: Fv fragments, single chain Fv (scFv) fragments, Fab′ fragments, F(ab′)2 fragments, single domain antibodies, humanized antibodies and antibody fragments, and multivalent versions of the foregoing. Multivalent targeting moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules that naturally interact with a desired target molecule. In certain embodiments, the antibodies or variants thereof, may be modified to make them less immunogenic when administered to a subject. For example, if the subject is human, the antibody may be “humanized”; where the complementarity determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. Also, transgenic mice, or other mammals, may be used to express humanized antibodies. Such humanization may be partial or complete. In certain embodiments, although the antibody is a murine or other non-human antibody, its humanness score is sufficient that humanization is not necessary. In still other embodiments, the antibody or antigen-binding fragment is fully human.

In some embodiments, the fusion protein does not comprise gastric inhibitory peptide (GIP) or a portion of GIP. For instance, the fusion protein may include no active fragments of GIP. In some embodiments, the fusion protein has fewer than 10, 8, or 6 contiguous amino acids of GIP.

In some embodiments, the fused portion is short. Thus, in some instances, the fusion protein comprises no more than 1, 2, 3, 4, 5, 10, or 20 additional amino acids on one or both termini of the polypeptide of Table 1 or homolog thereof.

The disclosure contemplates EDN3-like polypeptides and fusions thereof. Any such compounds can be readily tested using, for example, any one or more of the assays described herein. Moreover, any such compounds can be used in any of the methods described herein.

The disclosure contemplates that any of the EDN3-like polypeptides disclosed herein may be isolated and/or purified. For the avoidance of doubt, the term isolated does not include the mere presence of a transcript or other agent in a library—in the absence of any identification of the particular transcript or agent in the library or in the absence of steps to remove the transcript or agent of interest from other components of the library. The term purified, for example can be used to refer to a percentage purity, such as at least 80%, 85%, 90%, 91%, 92%, 93%, 94%, 95%, 96%, 97%, 98%, 99%, or even greater than 99% pure. Purity is typically represented as purity relative to the presence of other active agents in a composition. In other words, purity is typically used to indicate the substantial or significant absence of other active agents that might otherwise be considered a contaminant (rather than the mere presence of diluent, salt, buffer, or preservative).

2. Pharmaceutical Compositions Comprising EDN3-Like Polypeptides

Because EDN3-like polypeptides have useful pharmacological properties, this application discloses methods for preparing pharmaceutical compositions comprising EDN3-like polypeptides. Thus, in certain embodiments, compositions and polypeptides of the disclosure include compositions formulated in a pharmaceutically acceptable carrier. In certain embodiments, the pharmaceutical composition comprises an EDN3-like polypeptide and one or more of the following: a stabilizer, buffer, surfactant, controlled release component, salt, and a preservative. Any EDN3-like polypeptide, including any of the exemplary EDN3-like polypeptides disclosed herein, may be provided as a composition formulated in a pharmaceutically acceptable carrier.

In certain embodiments, the pharmaceutical composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In certain embodiments, the pharmaceutical composition includes one or more buffers such as a mixture of sodium bicarbonate and ascorbic acid. In some embodiments, the formulation may be administered in saline, such as phosphate buffered saline (PBS), or distilled water.

In certain embodiments, the pharmaceutical composition includes one or more surfactants such as polysorbate 80 (Tween 80), Triton X-100, Polyethylene glycol tert-octylphenyl ether t-Octylphenoxypolyethoxyethanol 4-(1,1,3,3-Tetramethylbutyl)phenyl-polyethylene glycol (TRITON X-100); Polyoxyethylenesorbitan monolaurate Polyethylene glycol sorbitan monolaurate (TWEEN 20); and 4-(1,1,3,3-Tetramethylbutyl)phenol polymer with formaldehyde and oxirane (TYLOXAPOL). A surfactant can be ionic or nonionic.

In certain embodiments, the pharmaceutical composition includes one or more salts such as sodium chloride, ammonium chloride, calcium chloride, or potassium chloride.

In certain embodiments, a preservative is included in the pharmaceutical composition. In other embodiments, no preservative is used. A preservative is most often used in multi-dose containers, and is less often needed in single-dose containers. In certain embodiments, the preservative is 2-phenoxyethanol, methyl and propyl parabens, benzyl alcohol, and/or sorbic acid.

In certain embodiments, the pharmaceutical composition is a controlled release formulation.

The pharmaceutical composition may be suitable for administration to a human patient, and pharmaceutical composition preparation may conform to USFDA guidelines. In some embodiments, the pharmaceutical composition is suitable for administration to a non-human animal. In some embodiments, the pharmaceutical composition is substantially free of either endotoxins or exotoxins. Endotoxins may include pyrogens, such as lipopolysaccharide (LPS) molecules. The pharmaceutical composition may also be substantially free of inactive protein fragments which may cause a fever or other side effects. In some embodiments, the composition contains less than 1%, less than 0.1%, less than 0.01%, less than 0.001%, or less than 0.0001% of endotoxins or exotoxins. In some embodiments, the pharmaceutical composition has lower levels of pyrogens than industrial water, tap water, or distilled water. Other pharmaceutical composition components may be purified using methods known in the art, such as ion-exchange chromatography, ultrafiltration, or distillation. In other embodiments, any pyrogens may be inactivated or destroyed prior to administration to a patient. Raw materials for the pharmaceutical compositions, such as water, buffers, salts and other chemicals may also be screened and depyrogenated. All materials in the pharmaceutical composition may be sterile, and each lot may be tested for sterility. Thus, in certain embodiments the endotoxin levels in the pharmaceutical composition fall below the levels set by the USFDA, for example 0.2 endotoxin (EU)/kg of product for an intrathecal injectable composition; 5 EU/kg of product for a non-intrathecal injectable composition, and 0.25-0.5 EU/mL for sterile water.

In certain aspects, an EDN3-like polypeptide in the pharmaceutical composition is a isolated protein. In general, the preparation may comprise less than 50%, 20%, 10%, or 5% (by dry weight) contaminating protein. In certain embodiments, the desired molecule (i.e., the EDN3-like polypeptide) is present in the substantial absence of other biological macromolecules, such as other proteins (particularly other proteins that substantially mask, diminish, confuse or alter the characteristics of the desired proteins either as isolated preparations or in their function in the subject reconstituted mixture). However, in some cases, a isolated protein may contain fragments of the desired protein (such as breakdown products or incomplete peptide synthesis products), as long as the fragments do not interfere substantially with the activity of the desired protein. In certain embodiments, at least 80%, 90%, 95%, 99%, or 99.8% (by dry weight) of biological macromolecules of the same type present (but water, buffers, and other small molecules, especially molecules having a molecular weight of less than 5000, can be present). In some embodiments, the composition contains less than 5%, 2%, 1%, 0.5%, 0.2%, 0.1% of protein from host cells in which the subunit proteins were expressed, relative to the amount of isolated subunit. Thus, in some instances, the pharmaceutical composition is substantially free of bacterial, yeast, or insect polypeptides. In some embodiments, the isolated protein contains substantially no other mammalian polypeptides other than an EDN3-like polypeptide. In some embodiments, the desired polypeptides are substantially free of nucleic acids and/or carbohydrates. For instance, in some embodiments, the composition contains less than 5%, less than 2%, less than 1%, less than 0.5%, less than 0.2%, or less than 0.1% host cell DNA and/or RNA. In certain embodiments, at least 80%, 90%, 95%, 99%, or 99.8% (by dry weight) of biological macromolecules of the same type are present in the preparation (but water, buffers, and other small molecules, especially molecules having a molecular weight of less than 5000, can be present).

It is preferred that the pharmaceutical composition has low or no toxicity, within a reasonable risk-benefit ratio. To quantify the toxicity of a pharmaceutical composition, LD₅₀ measurements may be obtained in mice or other experimental model systems, and extrapolated to humans and other animals. Methods for estimating the LD₅₀ of compounds in model organisms such as rats are well-known in the art. A pharmaceutical composition, and any component within it, might have an oral LD₅₀ value in rats of greater than 100 g/kg, greater than 50 g/kg, greater than 20 g/kg, greater than 10 g/kg, greater than 5 g/kg, greater than 2 g/kg, greater than 1 g/kg, greater than 500 mg/kg, greater than 200 mg/kg, greater than 100 mg/kg, or greater than 50 mg/kg.

The formulations suitable for introduction of the pharmaceutical composition vary according to route of administration. Formulations suitable for parenteral administration, such as, for example, by intraarticular (in the joints), intravenous, intramuscular, intradermal, intraperitoneal, intranasal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials.

The pharmaceutical forms suitable for injectable use include sterile aqueous solutions or dispersion and sterile powders for the extemporaneous preparation of sterile injectable solutions or dispersions. The form should be sterile and fluid to the extent that easy syringability exists.

Formulations suitable for oral administration can consist of (a) liquid solutions, such as an effective amount of the polypeptides or packaged nucleic acids suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. The pharmaceutical compositions can be encapsulated, e.g., in liposomes, or in a formulation that provides for slow release of the active ingredient.

The pharmaceutical compositions herein can be made into aerosol formulations (e.g., they can be “nebulized”) to be administered via inhalation. Aerosol formulations can be placed into pressurized acceptable propellants, such as dichlorodifluoromethane, propane, nitrogen, and the like. Aerosol formulations can be delivered orally or nasally.

This disclosure contemplates any EDN3-like polypeptide as disclosed herein (including polypeptides comprising or consisting of any of SEQ ID Nos. 1-33 and variants thereof) in combination with any of the pharmaceutically acceptable carriers or excipients described herein.

3. Methods of Producing EDN3-Like Polypeptides

In certain embodiments, this application provides for the synthesis of an EDN3-like polypeptide by solid phase protein synthesis. Diverse methods and systems for solid phase protein synthesis are known in the art. For example, one form is described by Merrifield (J. Am. Chem. Soc., 1963, 85:2149). More specifically, the synthesis is done in multiple steps by the Solid Phase Peptide Synthesis (SPPS) approach using Fmoc protected amino acids. SPPS is based on sequential addition of protected amino acid derivatives, with side chain protection where appropriate, to a polymeric support (bead). The base-labile Fmoc group is used for N-protection. After removing the protecting group (via piperidine hydrolysis) the next amino acid mixture is added using a coupling reagent (TBTU). After the final amino acid is coupled, the N-terminus can be acetylated. The resulting peptides (attached to the polymeric support through its C-terminus) are cleaved with TFA to yield the crude peptide. During this cleavage step, all of the side chain protecting groups are also cleaved. After precipitation with diisopropyl ether, the solid is filtered and dried. The resulting peptides can be analyzed and stored at 2-8° C.

In other embodiments, especially involving longer polypeptides, this application provides recombinant methods of protein production. Any suitable recombinant production method may be used, and several are well known in the art. Briefly, in recombinant production, a host cell expresses a nucleic acid encoding an EDN3-like polypeptide (or, where applicable, fusions comprising an EDN3-like portion). The host cell may be, for example, bacterial (e.g., E. coli), yeast, insect, or mammalian. A gene encoding the EDN3-like polypeptide is typically placed in the context of a vector. The vector may exist separately from the genome of the host, as is the case with most high copy number bacterial plasmids, or may be integrated into the host genome. Numerous expression vectors are known in the art, and many are commercially available. Typical vectors include a promoter sequence (which may be inducible, repressible, or constitutive), a multiple cloning site, an origin of replication, and a polyadenylation site.

Accordingly, the present disclosure describes nucleic acid sequences (e.g., DNA and RNA sequences) encoding EDN3-like polypeptides (or, where applicable, fusions comprising an EDN3-like portion). Furthermore, the present disclosure provides nucleic acid sequences that are complementary to those described above, i.e., nucleic acid sequences of the same length, wherein the nucleic acid sequence permits perfect base pairing between the two complementary sequences. The present disclosure also provides nucleic acids that hybridize with high stringency to said nucleic acids. High stringency conditions may include a wash step of 0.2×SSC at 65° C. The DNA sequences encoding the polypeptides described above may be modified in ways that do not affect the sequence of the protein product. For instance, the DNA sequence may be codon-optimized to improve expression in a host such as E. coli, yeast, an insect cell line (e.g., using the baculovirus expression system), or a mammalian (e.g., human or Chinese Hamster Ovary) cell line.

Once host cell lines have been established, EDN3-like polypeptides (or, where applicable, fusions comprising an EDN3-like portion) can be isolated. If the EDN3-like polypeptides accumulate in the cell, typically the polypeptides will be harvested from a cell lysate, while secreted proteins are typically isolated from conditioned medium. Protein isolation techniques are well known in the art. Briefly, one may physically isolate a given protein based on physical characteristics such as size, hydrophobicity, or affinity for a particular binding partner such as an antibody.

In the context of fusions comprising an EDN3-like portion, such fusions may be made in the same cell as an in-frame fusion (in the presence or absence of an intervening linker). Alternatively, the two portions may be produced separately, such as in separate vectors and/or cells, and then joined chemically or recombinantly (in the presence or absence of a linker).

In some embodiments, the first polypeptide portion is produced separately from the second polypeptide portion, and then the two polypeptides are covalently linked. Heterobifunctional crosslinking agents are a suitable class of compounds for creating a non-peptide bond between two polypeptides. Preparing protein-conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using, for instance, N-ethylmaleimide (see Partis et al. (1983) J. Pro. Chem. 2:263). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75).

In some embodiments, disulfide bridges are formed between specific cysteines in the EDN3-like polypeptide. For instance, the peptide EDN3 97-140 typically contains a cysteine bridge between residues C1 and C15, and another between C3 and C11. Alternatively, cysteine bridges may be allowed to form randomly within a peptide. As another alternative, a peptide may be placed in reducing conditions that disrupt or prevent the formation of disulfide bridges.

In some embodiments, the C-terminus of the EDN3-like polypeptide is amidated, and amidation can promote the stability of a polypeptide. N-terminal amidation may be performed, for instance, by palladium cleavage (U.S. Pat. No. 7,462,690), by a CnBr/o-nitrophenylglycine amide/photolysis procedure (U.S. Pat. No. 6,251,635), by enzymatic peptidyl alpha-amidation (Engels, Protein Engineering, 1:195-199 (1987)), and peptidyldehydroalanine treatment (Patchornik and Sokolovsky, JACS, 86: 1206-1212 (1964)). Peptide C-terminal amidation can also be achieved by treatment with HF or TFMSA of MBHA resin (G. R. Matsueda, et al. (1981) Peptides, 2, 45), or by TFA cleavage of Rink Amide MBHA resin (U.S. Pat. No. 5,124,478).

This disclosure contemplates the production of any EDN3-like polypeptide as disclosed herein (including polypeptides comprising or consisting of any of SEQ ID Nos. 1-33 and variants thereof) using any of the production methods herein. One of skill in the art can readily select a suitable production method based on the structure of the desired EDN3-like polypeptide.

4. Antibodies Specific to EDN3-Like Polypeptides

In certain embodiments, the present disclosure provides antibodies specific to EDN3-like polypeptides and methods of using the antibodies. While such antibodies would have a broad variety of uses, one particularly important use is in immunostaining to identify warm-sensitive neurons in the hypothalamus. The warm sensitive neurons are critical in maintaining energy homeostasis and regulating metabolism. However, in the past, identification of these neurons was a laborious process requiring electrophysiological recording (Tabarean et al. “Electrophysiological properties and thermosensitivity of mouse preoptic and anterior hypothalamic neurons in culture.” Neuroscience. 2005; 135(2):433-49). EDN3 97-140 may be present in warm sensitive neurons and thus EDN3-like-specific antibodies may be used in immunostaining techniques to quickly and efficiently identify warm sensitive neurons. Two pieces of data suggest that EDN3 97-140 is present in warm sensitive neurons. First, the mRNA encoding EDN3 97-140 is detected there (Example 1). Second, exogenous EDN3 97-140 administered to the hypothalamus increases CBT and decreases RER, suggesting it may be acting on warm sensitive neurons within the hypothalamus. This result is consistent with the model that endogenous EDN3 97-140 is expressed in warm sensitive neurons and so can act as a marker for these neurons.

A number of methods may be used to produce antibodies to given antigens. Any suitable methods may be used to make such antibodies. To make a polyclonal antibody, one may inject an EDN3-like peptide, (optionally, with an adjuvant such as Freund's complete adjuvant) into a host animal (such as a mouse, rat, rabbit, or chicken), and then harvest sera from the animal. The polyclonal antibody may be purified by affinity purification. Alternatively, monoclonal antibodies may be made. For a monoclonal antibody, typically a mouse is immunized. B cells are harvested from the immunized mouse, and immortalized through fusion with human cancer cells. One then selects a clonal hybridoma line that produces an antibody with the desired specificity, e.g., by limiting dilution, followed by testing the antibody e.g., using Western blots. The resulting hybridomas may be cultured, and monoclonal antibodies may be harvested from the culture medium. Affinity purification may also be used to purify monoclonal antibodies. Alternatively, antibodies may be produced using phage display. The antibodies produced by the above methods may then be used to design scFv antibodies, Fab fragments, or other types of antibodies, and techniques for doing so are well known in the art. Once the sequence of a suitable antibody is identified, it can be produced recombinantly in host cells. Also contemplated is the use of well known methods for making chimeric, humanized and fully human antibodies.

In some embodiments, an antibody specific for an EDN3-like polypeptide does not substantially bind the full length protein (preproendothelin-3) from which EDN3 97-140 is derived. This specificity may be caused by any differences in protein conformation between an EDN3-like polypeptide and preproendothelin-3. For instance, an epitope that is available in an EDN3-like polypeptide may be sterically masked in preproendothelin-3. Alternatively, an epitope in EDN3-like polypeptide may be locked in an unfavorable conformation in preproendothelin-3, preventing the antibody from binding to the full length protein. If an antibody is specific for an EDN3-like polypeptide compared to preproendothelin-3, and the EDN3-like polypeptide has amino acid differences from wild-type EDN3 97-140 and hence from the corresponding portion of wild-type preproendothelin-3, differences in specificity could also be attributed to amino acid sequence differences between the EDN3-like polypeptide and preproendothelin-3.

In other embodiments, an antibody specific for an EDN3-like polypeptide also binds specifically to preproendothelin-3. We note that an antibody suitable for use herein may bind specifically to one or more EDN3-like polypeptides and, optionally, to preproendothelin-3. The ability to bind to multiple EDN3-like polypeptides and/or preproendothelin-3 does not indicate that the antibody is non-specific.

This disclosure contemplates the use of any EDN3-like polypeptide as disclosed herein (including polypeptides comprising or consisting of any of SEQ ID Nos. 1-33 and variants thereof) for use in raising any type of antibody as described herein, using any suitable antibody production method.

5. Methods of Treatment Using EDN3-Like Polypeptides

EDN3 97-140 acts on a variety of cell types including enteroendocrine cells (Example 6) and hepatic cells (Example 8). In addition, EDN3 97-140 affects respiratory exchange ratio (Example 3) and glucose tolerance (Example 4) in mice. Consequently, EDN3-like polypeptides are appropriate for use in treating a broad variety of diseases and disorders. The role of EDN3 97-140 in regulating temperature-sensitive neurons is seen in its ability to increase core body temperature (Example 3). Because an increase in body temperature can be produced by an increase in metabolism (i.e., burning additional calories), an EDN3-like polypeptide may be used to treat metabolic disorders such as obesity. More directly, the temperature-regulating effect of EDN3-like polypeptides allows their use in the treatment of hypothermia.

Exemplary metabolic diseases or disorders that may be treated according to the methods herein include type I diabetes or type II diabetes, insulin resistance, lipid metabolic disorders, hyperlipidemia, hypercholesterolemia, and fatty acid metabolism disorders. Certain disorders are discussed in more detail below.

Diabetes, also called diabetes mellitus, is characterized by high blood sugar or ketoacidosis, and is often associated with chronic, general metabolic abnormalities arising from a prolonged high blood sugar status or a decrease in glucose tolerance. Diabetes can be classified as type I (insulin-dependent) or type II (Non Insulin Dependent Diabetes Mellitus or NIDDM). The risk factors for diabetes include the following: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

Insulin resistance is a condition in which tissues (typically, muscle, adipose, and hepatic tissues) that are normally insulin-responsive develop an inability or decreased ability to take up blood glucose in response to insulin. In a patient, insulin resistance can be detected by the fasting glucose test, which measures basal levels of blood glucose. Fasting glucose levels of 100 to 125 mg/dL are typical of a patient with insulin resistance. In contrast, higher levels are typical of diabetic patients. An alternative diagnostic for insulin resistance is the oral glucose tolerance test, in which a glucose solution is administered orally to a patient, and blood glucose levels are measured shortly after. A blood glucose level between 140 and 199 mg/dL is typical in patients with insulin resistance in this diagnostic assay.

Other examples of metabolic disorders include obesity, metabolic syndrome, insulin-resistance syndromes, syndrome X, insulin resistance, high blood pressure, hypertension, high blood cholesterol (hypercholesterolemia), dyslipidemia, hyperlipidemia, atherosclerotic disease including stroke, coronary artery disease or myocardial infarction, hyperglycemia, hyperinsulinemia and/or hyperproinsulinemia, impaired glucose tolerance, and delayed insulin release, lipodystrophy, cholesterol related disorders, such as gallstones, cholescystitis and cholelithiasis, and gout. Also considered within the scope of metabolic diseases and disorders are complications arising from diabetes including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, and hypertensive nephrosclerosis.

In some embodiments, the method of treating a disease includes the administration of a therapeutically effective amount of an EDN3-like polypeptide to a patient in need thereof, wherein the disease, condition or disorder is selected from Type I diabetes, Type II diabetes mellitus, idiopathic type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, pancreatitis, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's disease, schizophrenia, impaired cognition, inflammatory bowel disease, ulcerative colitis, Crohn's disease, and irritable bowel syndrome.

The terms “treatment”, “treating”, and the like are used herein to generally mean obtaining a desired pharmacologic and/or physiologic effect. The effect may be prophylactic in terms of reducing the severity or delaying the onset of a disease, condition, or symptoms thereof, and/or may be therapeutic in terms of a partial or complete cure for a disease or condition and/or adverse effect attributable to the disease or condition. In some embodiments, the prophylactic treatment is effective to the extent that a patient receiving the treatment does not suffer from the disease during his or her lifetime. “Treatment” as used herein covers any treatment of a disease or condition of a mammal, particularly a human, and includes: (a) reducing the likelihood that the disease or condition occurs in a subject that may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any of these conditions can be readily assessed according to standard methods and techniques known in the art. The population of subjects treated by the methods herein includes subjects suffering from the undesirable condition or disease, as well as subjects at risk for development of the condition or disease.

By the term “therapeutically effective dose” or “effective amount” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In certain embodiments, one or more EDN3-like polypeptides (including fusions) can be administered, together (simultaneously) or at different times (sequentially). Regardless of whether one EDN3-like polypeptide (including fusions) or multiple polypeptides are administered, in certain embodiments, polypeptides are administered in multiple doses. For example, in certain embodiments, treatment comprises administration more than once according to a schedule (e.g., daily, weekly, as needed, etc.).

The EDN3-like polypeptides herein may also be used to elevate energy expenditure. Energy expenditure (VO₂, VCO₂ and heat formation ((3.815+1.232*RER)*VO₂ (in liters)) can be measured in the mouse model using a respiratory chamber as described in Example 3. Simply put, it is a measurement, in calories, of all the energy an organism uses daily for all voluntary and involuntary functions of the body. The measurement is typically made in an environment without temperature extremes. It includes the amount of energy necessary to support the vital organs and maintain a normal body temperature. Energy expenditure can be measured by either direct or indirect calorimetry, but an estimated value can also be calculated by taking into account a subject's body surface area, which can be inferred from the subject's height and weight. In some embodiments, the EDN3-like polypeptides herein reduce the respiratory exchange ratio by at least 1%, 2%, 5%, 10%, 15%, or 20%.

Furthermore, EDN3-like polypeptides may be used to inhibit glucose production in hepatocytes, either in vivo or in vitro. In vitro, glucose production in hepatocytes may be measured according to the assay in Example 8. In an in vitro assay, primary or transformed hepatocytes may be used. In some embodiments, glucose production is lowered by at least 10%, 20%, 30%, 40%, or 50%.

In some embodiments, EDN3-like polypeptides may be used to promote GLP-1 secretion, either in vivo or in vitro. In vitro, GLP-1 production by enteric cells may be measured according to the GLUTag assay in Example 5. In some embodiments, the peptides stimulate GLP-1 secretion with an EC₅₀ of less than 100 nM, 200 nM, 300 nM, 400 nM, 500 nM, 1 μM, 2 μM, 5 μM, or 10 μM.

In some embodiments, EDN3-like polypeptides may be used to increase the core body temperature of a subject. In some instances, the polypeptide increases the core body temperature by an average of at least 0.25° C., 0.5° C., 0.75° C., 1° C., 1.25° C., 1.5° C., 1.75° C., 2° C., 2.25° C., 2.5° C., 2.75° C., or 3° C.

The EDN3-like polypeptides described herein are useful in treating various subjects, particularly human subjects but also including other mammals such as companion animals (including dogs and cats) and livestock (including cows and pigs). Subjects in need of treatment with an EDN3-like polypeptide include subjects with a metabolic disease or disorder as listed above, and also a subject judged to be at risk of developing such a disease or disorder. For example, a family history of a metabolic disease (including hypercholesterolemia and diabetes) indicates that a subject is at increased risk of developing the disease. Genetic markers can also indicate increased risk of a metabolic disease or disorder. In certain embodiments, administration of the EDN3-like polypeptide reduces the risk of a metabolic disease or disorder in a subject. Such a reduction in risk may be seen, for example, when a group of subjects treated with an EDN3-like polypeptide is compared with a group of control subjects over time, and the group of treated subjects shows a reduced incidence of the metabolic disease in comparison to the control subjects.

Treatment of a subject with EDN3-like polypeptides may result in the amelioration of at least one symptom of a metabolic disease or disorder. In some instances the symptom is an elevated resting blood glucose level. In some embodiments, the symptom is a higher-than-optimal body weight or body mass index.

This disclosure contemplates the use of any EDN3-like polypeptide, including any of the exemplary EDN3-like polypeptides disclosed herein (including polypeptides comprising or consisting of any of SEQ ID Nos. 1-33 and variants thereof, such as polypeptides of less than or equal to 60 amino acid residues and comprising any of SEQ ID Nos. 1-33) for use in treating any of the indications discussed herein.

6. Combination Therapies Comprising EDN3-Like Polypeptides

The EDN3-like polypeptides disclosed herein can be administered on their own, or can be administered together with an additional pharmaceutical agent, wherein the additional pharmaceutical agent treats one or more diseases that the EDN3-like polypeptide also treats. In some embodiments, the EDN3-like polypeptide and the additional pharmaceutical agent act additively or synergistically by treating the same symptom. In some embodiments, the EDN3-like polypeptide and the additional pharmaceutical agent complement each other by treating different symptoms.

Thus, in some embodiments, the pharmaceutical composition comprising an EDN3-like polypeptide includes or is administered in combination with at least one additional pharmaceutical agent selected from the group consisting of an anti-obesity agent, an anti-diabetic agent, an anti-hyperglycemic agent, a lipid lowering agent, and an anti-hypertensive agent. In another embodiment, the EDN3-like polypeptide and additional pharmaceutical agents are administered simultaneously, for instance as part of one pharmaceutical composition. In yet another embodiment, the EDN3-like polypeptide and additional pharmaceutical agents are administered sequentially in any order.

Lipid lowering agents include lipase inhibitors, NPY receptor antagonists, LDL-cholesterol lowering agents, triglyceride lowering agents, HMG-CoA reductase inhibitors, cholesterol synthesis inhibitors, cholesterol absorption inhibitors, CETP inhibitors, PPAR modulators or other cholesterol lowering agents such as a fibrate, niacin, an ion-exchange resin, an antioxidant, an ACAT inhibitor or a bile acid sequestrant. Other pharmaceutical agents useful in the combination therapies herein include bile acid reuptake inhibitors, ileal bile acid transporter inhibitors, ACC inhibitors, antihypertensive agents (such as amlodipine, e.g., Norvasc®), antibiotics, antidiabetics (such as metformin), PPAR-γ activators, sulfonylureas, insulin, aldose reductase inhibitors (AR¹) (e.g., zopolrestat), sorbitol dehydrogenase inhibitors (SDI)), and anti-inflammatory agents such as aspirin or, preferably, an anti-inflammatory agent that inhibits cyclooxygenase-2 (Cox-2) to a greater extent than it inhibits cyclooxygenase-1 (Cox-1) such as celecoxib (U.S. Pat. No. 5,466,823), valdecoxib (U.S. Pat. No. 5,633,272, parecoxib (U.S. Pat. No. 5,932,598), deracoxib (CAS RN 169590-41-4), etoricoxib (CAS RN 202409-33-4) or lumiracoxib (CAS RN 220991-20-8).

Lipase inhibitors are useful in the combination therapies herein. Lipase inhibitors inhibit the metabolic cleavage of dietary triglycerides into free fatty acids and monoglycerides. Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a monoglyceride and a fatty acid. The resultant free fatty acids and monoglycerides are incorporated into bile acid-phospholipid micelles, which are subsequently absorbed at the level of the brush border of the small intestine. The micelles eventually enter the peripheral circulation as chylomicrons. Lipase inhibition activity is readily determined by the use of standard assays well known in the art. See, for example, Methods Enzymol. 286: 190-231, incorporated herein by reference.

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors of this lipase find utility in the treatment of obesity and associated conditions.

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams et al., Gastroenterology, 92, 125 (1987).

A variety of pancreatic lipase inhibitors useful in the combination therapies are described hereinbelow. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin, (2S,3S,55)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. Tetrahydrolipstatin may be prepared as described in U.S. Pat. Nos. 5,274,143; 5,420,305; 5,540,917; and 5,643,874. The pancreatic lipase inhibitor FL-386, 1-[4-(2-methylpropyl)cyclohexyl]-2-[(phenylsulfonyl)oxy]-ethanone, and variously substituted sulfonate derivatives related thereto are disclosed in U.S. Pat. No. 4,452,813. The pancreatic lipase inhibitor WAY-121898, which is 4-phenoxyphenyl-4-methylpiperidin-1-yl-carboxylate, and various carbamate esters and pharmaceutically acceptable salts related thereto are disclosed in U.S. Pat. Nos. 5,512,565, 5,391,571, and 5,602,151. The pancreatic lipase inhibitor valilactone and a process for preparing it by microbial cultivation of Actinomycetes strain MG147-CF2 are disclosed in Kitahara et al., J. Antibiotics, 40 (11), 1647-1650 (1987). The pancreatic lipase inhibitors ebelactone A and ebelactone B and processes for preparing them by microbial cultivation of Actinomycetes strain MG7-G1 are disclosed in Umezawa et al., J. Antibiotics, 33, 1594-1596 (1980). The use of ebelactones A and B in the suppression of monoglyceride formation is disclosed in Japanese Kokai 08-143457, published Jun. 4, 1996. All of the references cited above are incorporated herein by reference.

Some appropriate lipase inhibitors include lipstatin, tetrahydrolipstatin, valilactone, esterastin, ebelactone A, and ebelactone B, particularly tetrahydrolipstatin. The lipase inhibitor N-3-trifluoromethylphenyl-N′-3-chloro-4′-trifluoromethylphenylurea, and the various urea derivatives related thereto are disclosed in U.S. Pat. No. 4,405,644. Esteracin is disclosed in U.S. Pat. Nos. 4,189,438 and 4,242,453. The lipase inhibitor cyclo-O,O′-[(1,6-hexanediyl)-bis-(iminocarbonyl)]dioxime and the various bis(iminocarbonyl)dioximes related thereto may be prepared as described in Petersen et al., Liebig's Annalen, 562, 205-229 (1949). All of the references cited above are incorporated herein by reference.

Preferred NPY receptor antagonists include NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345 and 6,326,375; U.S. Patent Application Publication Nos. 2002/0151456 and 2003/036652 and PCT Patent Application Publication Nos. WO 03/010175; WO 03/082190 and WO 02/048152.

A slow-release form of niacin is commercially available under the brand name Niaspan. Niacin may also be combined with other therapeutic agents such as lovastatin, which is an HMG-CoA reductase inhibitor. This combination therapy is sold under the trademark Advicor® (Kos Pharmaceuticals Inc).

Any HMG-CoA reductase inhibitor may be used as the additional compound in the combination therapies herein. The term HMG-CoA reductase inhibitor refers to compounds that inhibit the bioconversion of hydroxymethylglutaryl-coenzyme A to mevalonic acid catalyzed by the enzyme HMG-CoA reductase. Assays for determining are known in the art (e.g., Meth. Enzymol. 1981; 71:455-509 and references cited therein). HMG-CoA reductase inhibitors of interest herein include those disclosed in U.S. Pat. No. 4,231,938 (compounds isolated after cultivation of a microorganism belonging to the genus Aspergillus, such as lovastatin), U.S. Pat. No. 4,444,784 (synthetic derivatives of the aforementioned compounds such as simvastatin), U.S. Pat. No. 4,739,073 (substituted indoles such as fluvastatin), U.S. Pat. No. 4,346,227 (ML-236B derivatives such as pravastatin), European Patent Application Publication No. 491 226 A (pyridyldihydroxyheptenoic acids such as cerivastatin), U.S. Pat. No. 5,273,995 (6-[2-(substituted-pyrrol-1-yl)alkyl]pyran-2-ones such as atorvastatin and pharmaceutically acceptable forms thereof (i.e., atorvastatin, e.g., Lipitor®)). Additional HMG-CoA reductase inhibitors of interest herein include rosuvastatin and pitavastatin. All of the references cited above are incorporated herein by reference.

Preferred HMG-CoA reductase inhibitors include lovastatin, simvastatin, pravastatin, fluvastatin, atorvastatin or rivastatin; more preferably, atorvastatin, particularly atorvastatin hemicalcium.

Any compound having activity as a CETP inhibitor can serve as the additional compound in the combination therapies herein. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). CETP inhibitors useful in the combination therapies herein include those disclosed in U.S. Pat. Nos. 6,140,343 and 6,197,786. CETP inhibitors disclosed in these patents include compounds such as [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. Also of interest are the CETP inhibitors disclosed in U.S. Patent Application Pub. No. 2004-0204450, filed Mar. 23 2004 and its priority document U.S. Pat. App. Ser. No. 60/458,274, filed Mar. 28, 2003, U.S. Pat. No. 5,512,548 (polypeptide derivatives), J. Antibiot., 49(8): 815-816 (1996) (rosenonolactone derivatives) and Bioorg. Med. Chem. Lett.; 6:1951-1954 (1996) (phosphate-containing analogs of cholesteryl ester). All of the references cited above are incorporated herein by reference.

Any PPAR modulator may be used as the additional compound in the combination therapies herein. The term PPAR modulator refers to compounds which modulate peroxisome proliferator activator receptor (PPAR) activity in mammals, particularly humans. Such modulation may be readily determined by standard assays known in the art. It is believed that such compounds, by modulating the PPAR receptor, stimulate transcription of key genes involved in fatty acid oxidation and genes involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components and increase HDL cholesterol and apolipoprotein Al. Hence, these compounds are useful for the treatment and correction of the various dyslipidemias associated with the development and incidence of atherosclerosis and cardiovascular disease, including hypoalphalipoproteinemia and hypertriglyceridemia. PPAR-α activators of interest herein include those disclosed in PCT Patent Application Publication Nos. WO 02/064549 and WO 02/064130 and U.S. patent application Ser. No. 10/720,942 (published as 2004-0157885), filed Nov. 24, 2003. All of the references cited above are incorporated herein by reference.

Any HMG-CoA synthase inhibitor may be used as the additional compound in the combination therapies herein. The term HMG-CoA synthase inhibitor refers to compounds that inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by standard assays known in the art. Enzymol. 1975; 35:155-160: Meth. Enzymol. 1985; 110:19-26 and references cited therein). HMG-CoA synthase inhibitors of interest include those disclosed in U.S. Pat. No. 5,120,729 (beta-lactam derivatives), U.S. Pat. No. 5,064,856 (spiro-lactone derivatives prepared by culturing a microorganism (MF5253)) and U.S. Pat. No. 4,847,271 (certain oxetane compounds such as 11-(3-hydroxymethyl-4-oxo-2-oxetayl)-3,5,7-trimethyl-2,4-undeca-dienoic acid derivatives). All of the references cited above are incorporated herein by reference.

Any compound that decreases HMG-CoA reductase gene expression may be used as the additional compound in the combination therapies herein. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such regulation is readily determined by those skilled in the art according to standard assays (Meth. Enzymol., 1985; 110:9-19). U.S. Pat. No. 5,041,432 discloses certain 15-substituted lanosterol derivatives that decrease HMG-CoA reductase gene expression. Other oxygenated sterols that suppress synthesis of HMG-CoA reductase are discussed by E.I. Mercer (Prog. Lip. Res. 1993; 32:357-416). The references cited above are incorporated herein by reference.

Squalene synthetase inhibitors are also useful as the additional compound in the combination therapies herein. Such compounds inhibit the condensation of 2 molecules of farnesylpyrophosphate to form squalene, catalyzed by the enzyme squalene synthetase. Standard assays for determining squalene synthetase inhibition are well known in the art. (Meth. Enzymol. 1969; 15: 393-454 and Meth. Enzymol. 1985; 110:359-373 and references contained therein.) Squalene synthetase inhibitors of interest herein include those disclosed in U.S. Pat. No. 5,026,554 (fermentation products of the microorganism MF5465 (ATCC 74011) including zaragozic acid) as well as those included in the summary of patented squalene synthetase inhibitors which appears in Curr. Op. Ther. Patents (1993) 861-4. The references cited above are incorporated herein by reference.

Any squalene epoxidase inhibitor may be used as the additional compound in the combination therapies herein. These compounds inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). Squalene epoxidase inhibitors of interest herein include those disclosed in U.S. Pat. Nos. 5,011,859 and 5,064,864 (fluoro analogs of squalene), European Patent Application Publication No. 395,768 A (substituted allylamine derivatives), PCT Patent Application Publication No. WO 93/12069 A (amino alcohol derivatives) and U.S. Pat. No. 5,051,534 (cyclopropyloxy-squalene derivatives). All of the references cited above are incorporated herein by reference.

Squalene cyclase inhibitors are also contemplated herein as the additional pharmaceutical agent for use in the combination therapies herein. These compounds inhibit the bioconversion of squalene-2,3-epoxide to lanosterol, catalyzed by the enzyme squalene cyclase. Such inhibition is readily determined by standard assays well known in the art. (FEBS Lett. 1989; 244:347-350). Squalene cyclase inhibitors of interest include those disclosed in PCT Patent Application Publication No. WO 94/10150 (1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine) and French Patent Application Publication No. 2697250 (beta, beta-dimethyl-4-piperidine ethanol derivatives such as 1-(1,5,9-trimethyldecyl)-beta, beta-dimethyl-4-piperidineethanol). The references cited above are incorporated herein by reference.

Any combined squalene epoxidase/squalene cyclase inhibitor may be used as the additional pharmaceutical agent in the combination therapies. The term combined squalene epoxidase/squalene cyclase inhibitor refers to compounds that inhibit the bioconversion of squalene to lanosterol via a squalene-2,3-epoxide intermediate. Combined squalene epoxidase/squalene cyclase inhibition is readily determined in standard assays for squalene cyclase inhibitors or squalene epoxidase inhibitors. Squalene epoxidase/squalene cyclase inhibitors useful in the combination therapies herein include those disclosed in U.S. Pat. Nos. 5,084,461 and 5,278,171 (azadecalin derivatives), European Patent Application Publication No. 468,434 (piperidyl ether and thio-ether derivatives such as 2-(1-piperidyl)pentyl isopentyl sulfoxide and 2-(1-piperidyl)ethyl ethyl sulfide), PCT Patent Application Publication No. WO 94/01404 (acyl-piperidines such as 1-(1-oxopentyl-5-phenylthio)-4-(2-hydroxy-1-methyl)-ethyl)piperidine) and U.S. Pat. No. 5,102,915 (cyclopropyloxy-squalene derivatives). All of the references cited above are incorporated herein by reference.

The EDN3-like polypeptides can also be administered in combination with naturally occurring substances that act to lower plasma cholesterol levels. These naturally occurring materials are commonly called nutraceuticals and include, for example, garlic extract, Hoodia plant extracts and niacin.

Cholesterol absorption inhibitors may also be used in the combination therapies herein. The term cholesterol absorption inhibition refers to the ability of a compound to prevent cholesterol contained within the lumen of the intestine from entering into the intestinal cells and/or passing from within the intestinal cells into the blood stream. Such cholesterol absorption inhibition activity is readily determined in standard assays (e.g., J. Lipid Res. (1993) 34: 377-395). Cholesterol absorption inhibitors of interest include those disclosed in PCT Patent Application Publication No. WO 94/00480. A preferred cholesterol absorption inhibitor is ezetimibe, e.g., Zetia™ (Merck/Schering-Plough). The references cited above are incorporated herein by reference.

Any ACAT inhibitor may serve as the additional pharmaceutical agent in the combination therapies herein. The term ACAT inhibitor refers to compounds that inhibit the intracellular esterification of dietary cholesterol by the enzyme acyl CoA: cholesterol acyltransferase. Such inhibition may be determined by standard assays, such as the method of Heider et al. described in Journal of Lipid Research., 24:1127 (1983). ACAT inhibitors useful herein include those disclosed in U.S. Pat. No. 5,510,379 (carboxysulfonates) and PCT Patent Application Publication Nos. WO 96/26948 and WO 96/10559 (both disclose urea derivatives). Preferred ACAT inhibitors include avasimibe (Pfizer), CS-505 (Sankyo) and eflucimibe (Eli Lilly and Pierre Fabre). All of the references cited above are incorporated herein by reference.

Other compounds that are marketed for hyperlipidemia, including hypercholesterolemia, and which are intended to help prevent or treat atherosclerosis and are of interest herein include bile acid sequestrants, such as Colesevelam, e.g., Welchol®, Colestipol, e.g., Colestid®, Cholestyramine Resin e.g., LoCholest®, Cholestyramine, e.g., Questran®; and fibric acid derivatives, such as Clofibrate, e.g., Atromid®, Gemfibrozil, e.g., Lopid® and Fenofibrate, e.g., Tricor®.

Diabetes (especially Type II), insulin resistance, impaired glucose tolerance, or the like, and any of the diabetic complications such as neuropathy, nephropathy, retinopathy or cataracts may be treated by the administration of a therapeutically effective amount of an EDN3-like polypeptide in combination with one or more additional pharmaceutical agents (e.g., insulin) that are useful in treating diabetes.

Any glycogen phosphorylase inhibitor may be used as the additional agent in combination with an EDN3-like polypeptide. The term glycogen phosphorylase inhibitor refers to compounds that inhibit the bioconversion of glycogen to glucose-1-phosphate, which is catalyzed by the enzyme glycogen phosphorylase. Such glycogen phosphorylase inhibition activity is readily determined by standard assays well known in the art (e.g., J. Med. Chem. 41 (1998) 2934-2938). Glycogen phosphorylase inhibitors of interest herein include those described in PCT Patent Application Publication Nos. WO 96/39384 and WO 96/39385. The references cited above are incorporated herein by reference.

Aldose reductase inhibitors are also useful in the combination therapies herein. These compounds inhibit the bioconversion of glucose to sorbitol, which is catalyzed by the enzyme aldose reductase. Aldose reductase inhibition is readily determined by standard assays (e.g., J. Malone, Diabetes, 29:861-864 (1980) “Red Cell Sorbitol, an Indicator of Diabetic Control”, incorporated herein by reference). A variety of aldose reductase inhibitors are known to those skilled in the art. The reference cited above is incorporated herein by reference.

Any sorbitol dehydrogenase inhibitor may be used in combination with an EDN3-like polypeptide. The term sorbitol dehydrogenase inhibitor refers to compounds that inhibit the bioconversion of sorbitol to fructose, which is catalyzed by the enzyme sorbitol dehydrogenase. Such sorbitol dehydrogenase inhibitor activity is readily determined by the use of standard assays well known in the art (e.g., Analyt. Biochem (2000) 280: 329-331). Sorbitol dehydrogenase inhibitors of interest include those disclosed in U.S. Pat. Nos. 5,728,704 and 5,866,578. The references cited above are incorporated herein by reference.

Any glucosidase inhibitor can be used in the combination therapies herein. Such compounds inhibit the enzymatic hydrolysis of complex carbohydrates by glycoside hydrolases such as amylase or maltase into bioavailable simple sugars, for example, glucose. The rapid metabolic action of glucosidases, particularly following the intake of high levels of carbohydrates, results in a state of alimentary hyperglycemia, which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known that chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969)8: 4214), incorporated herein by reference.

One appropriate type of glucosidase inhibitor is an amylase inhibitor. An amylase inhibitor is a glucosidase inhibitor that inhibits the enzymatic degradation of starch or glycogen into maltose. Such amylase inhibition activity is readily determined by use of standard assays (e.g., Methods Enzymol. (1955)1: 149, incorporated herein by reference). The inhibition of such enzymatic degradation is beneficial in reducing amounts of bioavailable sugars, including glucose and maltose, and the concomitant deleterious conditions resulting therefrom.

Certain appropriate glucosidase inhibitors include acarbose, adiposine, voglibose, miglitol, emiglitate, camiglibose, tendamistate, trestatin, pradimicin-Q and salbostatin. The glucosidase inhibitor acarbose and various amino sugar derivatives related thereto are disclosed in U.S. Pat. Nos. 4,062,950 and 4,174,439 respectively. The glucosidase inhibitor adiposine is disclosed in U.S. Pat. No. 4,254,256. The glucosidase inhibitor voglibose, 3,4-dideoxy-4-[[2-hydroxy-1-(hydroxymethyl)ethyl]amino]-2-C-(hydroxymethyl-I)-D-epi-inositol, and various N-substituted pseudo-aminosugars related thereto are disclosed in U.S. Pat. No. 4,701,559. The glucosidase inhibitor miglitol, (2R,3R,4R,5S)-1-(2-hydroxyethyl)-2-(hydroxymethyl)-3,4,5-piperidinetriol, and various 3,4,5-trihydroxypiperidines related thereto are disclosed in U.S. Pat. No. 4,639,436. The glucosidase inhibitor emiglitate, ethyl p[2-[(2R,3R,4R,5S)-3,4,5-trihydroxy-2-(hydroxymethyl)piperidino]ethoxy]-benzoate, various derivatives related thereto and pharmaceutically acceptable acid addition salts thereof are disclosed in U.S. Pat. No. 5,192,772. The glucosidase inhibitor MDL-25637, 2,6-dideoxy-7-O-.beta.-D-glucopyrano-syl-2,6-imino-D-glycero-L-gluco-heptitol, various homodisaccharides related thereto and the pharmaceutically acceptable acid addition salts thereof are disclosed in U.S. Pat. No. 4,634,765. The glucosidase inhibitor camiglibose, methyl 6-deoxy-6-[(2R,3R,4R,5S)-3,4,5-trihydroxy-2-(hydroxymethyl)piperidino]-α-D-glucopyranoside sesquihydrate, deoxy-nojirimycin derivatives related thereto, various pharmaceutically acceptable salts thereof and synthetic methods for the preparation thereof are disclosed in U.S. Pat. Nos. 5,157,116 and 5,504,078. The glycosidase inhibitor salbostatin and various pseudosaccharides related thereto are disclosed in U.S. Pat. No. 5,091,524. All of the references cited above are incorporated herein by reference.

Amylase inhibitors of interest herein are disclosed in U.S. Pat. No. 4,451,455, U.S. Pat. No. 4,623,714 (AI-3688 and the various cyclic polypeptides related thereto) and U.S. Pat. No. 4,273,765 (trestatin, which consists of a mixture of trestatin A, trestatin B and trestatin C, and the various trehalose-containing amino sugars related thereto). All of the references cited above are incorporated herein by reference.

Additional anti-diabetic compounds, which may be used as the additional pharmaceutical agent in combination with the EDN3-like polypeptides, include, for example, the following: biguanides (e.g., metformin, pfenformin or buformin), insulin secretagogues (e.g., sulfonylureas and glinides), glitazones, non-glitazone PPAR-γ agonists, PPAR-β agonists, inhibitors of DPP-IV (i.e., sitagliptin, vilagliptin, saxagliptin, linagliptin, alogliptin, and berberine), inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-β inhibitors and AGE breakers.

The EDN3-like polypeptides may also be used in combination with antihypertensive agents. Appropriate antihypertensive agents useful in the combination therapies herein include calcium channel blockers, such as Diltiazem (e.g., Cardizeme®, Dilacor®, or Tiazac®), Nifedipine (e.g., Adalat® or Procardia XL®), Verapamil (e.g., Calan®, Verelan®, or Isoptin®), Nicardipine (e.g., Cardene®), Verapamil (e.g., Covera® (e.g., Isradipine (e.g., DynaCirc®), Nisoldipine (e.g., Sular®), Bepridil (e.g., Vascor®), Nimodipine (e.g., Nimotop®), Amlodipine (e.g., Norvasc®), and Felodipine (e.g., Plendil®); angiotensin converting enzyme (ACE) inhibitors, such as Quinapril (e.g., Accupril®), Ramipril (e.g., Altace®), Captopril (e.g., Capoten®), Benazepril (e.g., Lotensin®), Trandolapril (e.g., Mavik®), Fosinopril (e.g., Monopril®), Lisinopril (e.g., Prinivil® or Zestril®), Moexipril (e.g., Univasc®), and Enalapril (e.g., Vasotec®).

The additional pharmaceutical agent may be, for instance, an anti-obesity agent or anti-diabetic agent as described above, and can also be an HMG-CoA reductase inhibitor, an HMG-CoA synthase inhibitor, an inhibitor of HMG-CoA reductase gene expression, a CETP inhibitor, a PPAR modulator, a squalene synthetase inhibitor, a squaline epoxidase inhibitor, a squaline cyclase inhibitor, a combined squaline epoxidase/cyclase inhibitor, a cholesterol absorption inhibitor, an ACAT inhibitor, a pancreatic lipase inhibitor, a gastric lipase inhibitor, a calcium channel blocker, an ACE inhibitor, a beta blocker, a diuretic, niacin, a garlic extract preparation, a bile acid sequestrant, a fibric acid derivative, a glycogen phosphorylase inhibitor, an aldose reductase inhibitor, a sorbitol dehydrogenase inhibitor, an SGLT2 inhibitor (i.e., dapagliflozin, BI-10773, and the compounds disclosed in WO2010/023594 filed on Aug. 17, 2009), a glucosidase inhibitoran amylase inhibitor or a DPP-IV inhibitor (i.e., sitagliptin, vilagliptin, saxagliptin, linagliptin, alogliptin, and berberine).

The dosage of the additional pharmaceutical agent is generally dependent upon a number of factors including the health of the subject being treated, the extent of treatment desired, the nature and kind of concurrent therapy, if any, and the frequency of treatment and the nature of the effect desired. In general, the dosage range of the additional pharmaceutical agent is in the range of from about 0.001 mg to about 100 mg per kilogram body weight of the individual per day, for instance from about 0.1 mg to about 10 mg per kilogram body weight of the individual per day. However, some variability in the general dosage range may also be required depending upon the age and weight of the subject being treated, the intended route of administration, the particular additional therapeutic agent being administered and the like. The determination of dosage ranges and optimal dosages for a particular patient is also well within the ability of one of ordinary skill in the art having the benefit of the instant disclosure.

In certain embodiments, the method of treatment comprises a combination therapy in which an EDN3-like polypeptide is administered with one or more additional pharmaceutical agents. In the combination therapies herein, the EDN3-like polypeptide and at least one other pharmaceutical agent (e.g., an anti-obesity agent or anti-diabetic agent) may be administered either separately or in a pharmaceutical composition comprising both. In some embodiments, the administration is oral.

When a combination of an EDN3-like polypeptide and at least one other pharmaceutical agent are administered together, such administration may be sequential in time or simultaneous. For sequential administration, an EDN3-like polypeptide and the additional pharmaceutical agent may be administered in any order. In some embodiments, the administration is oral and simultaneous. When an EDN3-like polypeptide and the additional pharmaceutical agent are administered sequentially, the administration of each may be by the same or by different routes of administration.

This disclosure contemplates the use of any EDN3-like polypeptide, including any of the exemplary EDN3-like polypeptides disclosed herein (including polypeptides comprising or consisting of any of SEQ ID Nos. 1-33 and variants thereof, such as polypeptides of less than or equal to 60 amino acid residues and comprising any of SEQ ID Nos. 1-33) for use in combination with any of the combination therapeutics described herein.

7. Other Uses of EDN3-Like Polypeptides

The EDN3-like polypeptides disclosed herein are useful not only in treating disease, but also in various areas of research. For example, in the field of temperature homeostasis, researchers sometimes wish to perturb the core body temperature of experimental organisms in order to determine the effect of core body temperature on a particular pathway or protein. EDN3-like polypeptides can be used to experimentally manipulate core body temperature for this purpose. In other instances, researchers studying GLP-1 may wish to study the effects of elevated GLP-1 levels on a particular pathway or protein in a model organism. EDN3-like polypeptides can be used to experimentally elevate GLP-1 levels for this purpose. As another example, researchers studying metabolism may wish to experimentally elevate or depress energy expenditure in a model organism in order to observe the effects at the organismal or molecular level. EDN3-like polypeptides can be used to experimentally elevate energy expenditure for this purpose. In still other circumstances, researchers studying hepatocyte function may wish to stimulate or inhibit glucose production in hepatocytes in order to study the mechanism of glucose production in these cells. EDN3-like polypeptides can be used to experimentally inhibit glucose production for this purpose. In yet other circumstances, a researcher studying glucose metabolism may wish to promote or inhibit glucose uptake in various cell types (such as skeletal muscle cells or adipocytes) in order to study the cellular machinery responsible for glucose uptake. EDN3-like polypeptides can be used to experimentally elevate glucose uptake for this purpose. As these examples illustrate, uses of EDN3-like polypeptides include their use as reagents.

EDN3-like polypeptides are also useful in various imaging techniques. Because these polypeptides have affinity for particular receptors, they can be used to determine the distribution of such receptors in a patient's body. This application is especially useful in identifying gross overexpression of the receptor, as might be expected in a tumor. To be useful in an imaging assay, an EDN3-like polypeptide should comprise a label. The label may be, for example, a fluorescent label, a radiolabel, or an MRI-detectable label. A fluorescent label is typically detected with a CCD camera or other camera that detects visible light. A radiolabel can be detected with, for example, a gamma camera or radiosensitive film. An MRI-detectable label can be detected by magnetic resonance imaging or other NRM-based devices. Numerous examples of detectable labels are provided herein.

By way of example, a labeled EDN3-like polypeptide can be administered to a subject, and the distribution of the labeled EDN3-like polypeptide can be detected in the subject. By way of further example, a tissue sample taken from a subject can be contacted ex vivo with labeled EDN3-like polypeptide.

It is understood that this disclosure contemplates any EDN3-like polypeptide as disclosed herein (including polypeptides comprising or consisting of any of SEQ ID Nos. 1-33 and variants thereof) for use in the methods described in this section.

8. EDN3-Like RNA as a Marker of Warm Sensitive Neurons

As mentioned in Section 4 above, in the past identification of warm sensitive neurons was a laborious process requiring electrophysiological recording. The identification of EDN3 97-140 as a potential marker of warm sensitive neurons (Examples 1 and 3) suggests the use of EDN3 97-140-specific antibodies in immunostaining techniques to quickly and efficiently identify warm sensitive neurons. Thus, the present application provides a method of identifying warm-sensitive neurons, comprising contacting a brain tissue sample with an antibody that binds specifically to a polypeptide comprising the amino acid sequence of SEQ ID No. 19 or 21.

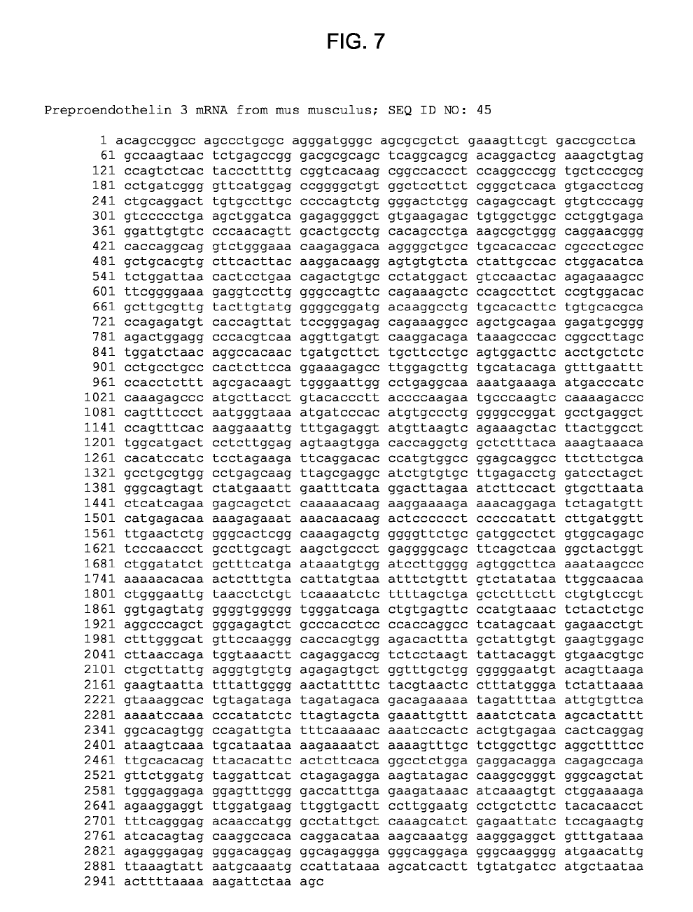

In addition, the fact that EDN3 mRNA is expressed in warm sensitive neurons (Example 1) allows one of skill in the art to identify warm sensitive neurons using in situ hybridization techniques with nucleic acid probes to EDN3 mRNA. Appropriate in situ hybridization techniques may use fluorescently labeled probes, or probes labeled with an enzymatic or radioactive activity. Such techniques are well known in the art. An appropriate probe to target the RNA encoding EDN3 97-140 may cover the coding region, non-coding region, or both. Because EDN3 97-140 is likely a cleavage product of the preproendothelin-3, a probe directed against any part of preproendothelin-3 mRNA can be used in the hybridization methods herein. The sequence of preproendothelin-3 in mice is provided at Gen Bank accession number NM_{—}007903, and is shown in FIG. 7 as SEQ ID NO: 45.

9. Animal Models for Assaying EDN3-Like Polypeptides

In Examples 5 and 8, this application provides useful cell culture models for EDN3-like polypeptide activity. These assays are excellent for rapidly screening large numbers of polypeptides. As a complimentary approach, animal models may be used to test EDN3-like activity on a systemic level. Using the well-known animal models described here, as well as other models known in the art, one of skill in the art can readily determine whether any EDN3-like polypeptide within the scope of the claims has activity in treating a metabolic disease.

To that end, this application describes the well-known diet-induced obesity (D10) mouse which is a model of obesity and diabetes (Example 4). Core body temperature and energy expenditure in the mouse can be measured according to the methods described in Example 3.

Useful animal models also exist for other metabolic disorders such as hyperlipidemia, hypercholesterolemia, and fatty acid metabolism disorders. These three disorders are often caused by diabetes, and some mouse models display more than one of these traits.

Hyperlipidemia can be studied in mice that overexpress the Lep(ob) gene (Soga M, “Insulin resistance, steatohepatitis, and hepatocellular carcinoma in a new congenic strain of Fatty Liver Shionogi (FLS) mice with the Lep(ob) gene.” Exp Anim. 2010; 59(4):407-19). When allowed to feed ad libitum, the mice develop severe hyperlipidemia over the course of 12 weeks. Serum triglycerides may be assayed using, for example, a colorimetric triglyceride assay kit produced by Eiken Chemical (Japan) under the trade name TRIGLYZIME.

Hypercholesterolemia can be studied in KK-Ay mice, an animal model of type 2 diabetes (Takagi S et al., “Effect of corosolic acid on dietary hypercholesterolemia and hepatic steatosis in KK-Ay diabetic mice.” Biomed Res. 2010; 31(4):213-8.) Briefly, feeding KK-Ay mice a high cholesterol diet causes the mice to develop hypercholesterolemia over the course of 10 weeks. One can administer test compounds to the mice to determine the effect on mean blood cholesterol levels.

Defects in fatty acid metabolism often result in perturbed levels of free fatty acids (FFAs) in the serum. Serum FFAs can be detected by liquid chromatography/mass spectrometry methods as described in Le Bouter et al. “Coordinate Transcriptomic and Metabolomic Effects of the Insulin Sensitizer Rosiglitazone on Fundamental Metabolic Pathways in Liver, Soleus Muscle, and Adipose Tissue in Diabetic db/db Mice” PAR Res. 2010; 2010: 679184. The db/db mouse develops type II diabetes, with correspondingly abnormal FFAs, as a result of a lesion in the leptin receptor gene.

This disclosure contemplates the administration of any EDN3-like polypeptide as disclosed herein (including polypeptides comprising or consisting of any of SEQ ID Nos. 1-33 and variants thereof) in the animal models described in this section.

10. In Vitro and Ex Vivo Assays for EDN3-Like Polypeptides

This application teaches one of skill in the art not only how to produce EDN-3 like polypeptides (Section 3 of the Detailed Description), but also how to assay their activities in vivo (Section 9 of the Detailed Description) and in vitro. Certain useful in vitro cell culture assays for EDN3-like activity are described in this section.

Specifically, EDN3-like polypeptides may have one or more of the following activities: (a) inhibiting glucose production in hepatocytes, (b) promoting GLP-1 secretion in the rat perfused colon assay, (c) promoting GLP-1 secretion in GLUTag cells, (d) promoting glucose uptake in skeletal muscle cells, or (e) promoting glucose uptake in adipocytes. In some instances, the EDN3-like polypeptides have one, two, three, four, or all five of the activities in (a)-(e). In some embodiments, the EDN3-like polypeptides have one, two, or three of the activities listed in (a)-(c).

This application discloses methods for assaying each of activities (a)-(e). For instance, Example 8 describes a suitable method for assaying activity (a), glucose production in hepatocytes. The protocol of Example 8 involves using an Amplex Red Glucose/Glucose Oxidase Assay Kit to determine the amount of glucose produced by cultured H4IIE cells. However, other suitable assays are known in the art. For example, Kim S J et al. (“Ginsenoside Rg1 suppresses hepatic glucose production via AMP-activated protein kinase in HepG2 cells” Biol Pharm Bull. 2010 February; 33(2):325-8) performs a similar assay in HepG2 cells. One of skill in the art could thus follow an assay disclosed in this application or in other publications to determine whether a given EDN3-like polypeptide inhibits gluconeogenesis in hepatocytes.

This application also discloses a method for assaying activity (b), promoting GLP-1 secretion in the rat perfused colon assay (Example 7). Briefly, a rat mesenteric artery and portal vein are obtained and perfused with a solution comprising the EDN3-like polypeptide of interest. Portal effluent is collected and assayed for active secreted GLP-1 using a commercially available Millipore kit. The rat perfused colon model system is also described, e.g., in Moro F et al. (“Release of guanylin immunoreactivity from the isolated vascularly perfused rat colon” Endocrinology. 2000 July; 141(7):2594-9). GLP-1 can be detected in a number of ways. GLP-1 protein levels can be detected by Western blot or ELISA, for example. Alternatively, GLP-1 activity can be determined by, for example, administering the GLP-1-containing sample to INS-1 cells and determining the change in cAMP levels by radioimmunoassay as described in Baggio L et al. (“Chronic Exposure to GLP-1R Agonists Promotes Homologous GLP-1 Receptor Desensitization In Vitro but Does Not Attenuate GLP-1R-Dependent Glucose Homeostasis In Vivo”, Diabetes December 2004 Col. 53 Supplement 3 5205-5214). One of skill in the art could thus follow an assay disclosed in this application or in other publications to determine whether a given EDN3-like polypeptide promotes GLP-1 secretion in, for example, the rat perfused colon assay.

This application also discloses a method for assaying activity (c), promoting GLP-1 secretion in GLUTag cells (Example 6). In brief, the GLUTag assay involves contacting GLUTag cells with an EDN3-like polypeptide, and then measuring the GLP-1 produced using the GLP-1 assay described in the previous paragraph. One of skill in the art could thus follow an assay disclosed in this application or in other publications to determine whether a given EDN3-like polypeptide promotes GLP-1 secretion in GLUTag cells.

In some embodiments, the EDN3-like polypeptide (d) promotes glucose uptake in skeletal muscle cells, or (e) promotes glucose uptake in adipocytes. Glucose uptake assays are readily available to one of skill in the art. For instance, one may measure glucose uptake in skeletal muscle cells by culturing the skeletal muscle cells, adding the EDN3-like polypeptide, and determining the amount of 2-deoxyglucose in the cell culture media by fluorescence, as described in Yamamoto N et al. (“Artemisia princeps extract promoted glucose uptake in cultured L6 muscle cells via glucose transporter 4 translocation.” Biosci Biotechnol Biochem. 2010 Oct. 23; 74(10):2036-42. Epub 2010 Oct. 7). Glucose uptake assays in adipocytes can be performed according to a similar protocol. For instance, one may measure glucose uptake in adipose cells by culturing the adipose cells (for instance 3T3-L1 cells), adding the EDN3-like polypeptide, and determining the amount of tritiated 2-deoxyglucose in the cell culture media by scintillation counting, as described in Fujita et al. (“Identification of three distinct functional sites of insulin-mediated GLUT4 trafficking in adipocytes using quantitative single molecule imaging.” Mol Biol Cell. 2010 Aug. 1; 21(15):2721-31). One of skill in the art could thus determine whether a given EDN3-like polypeptide promotes glucose uptake in muscle or adipose cells.

The disclosure recognizes that the EDN3-like polypeptides may have additional functions or activities in other assays. However, EDN3-like polypeptide for use as described herein may be readily identified based on their ability to have activity in one or more of the foregoing assays.

This disclosure contemplates the use of any EDN3-like polypeptide as disclosed herein (including polypeptides comprising or consisting of any of SEQ ID Nos. 1-33 and variants thereof) in the assays described in this section.

11. Receptors for EDN3-Like Polypeptides

Identifying the receptor or receptors that mediate EDN3-like activity is of considerable interest. The experiments in Example 5 suggest that the GLP-1-release activity of EDN3 97-140 is mediated by a G-protein coupled receptor. The receptor mediating EDN3 activity in the hypothalamus and in hepatic cells may be the same or different. Moreover, the different functional activities of EDN3-like polypeptides may be mediated by the same or different receptors. Accordingly, this application discloses methods for using EDN3-like polypeptides to identify the one or more receptors that mediate EDN3-like activity.

The respiratory exchange ratio (RER) model described in Example 3 can be used to identify the receptors in the hypothalamus that respond to EDN3-like polypeptides. As Example 3 shows, injection of EDN3 97-140 into the hypothalamus reduces the RER in mice. Accordingly, EDN3 97-140 or other EDN3-like polypeptides can be injected into the hypothalamus in the presence or absence of inhibitors of certain receptors, and the effect on the RER can be determined. For instance, CTX inhibits Gαₛ, so is indicative of a peptide that acts through a particular class of G-protein coupled receptors (Example 5). Numerous other hormone receptor inhibitors are known. To name a few, raloxifene is an estrogen receptor antagonist, pegvisomant is a growth hormone receptor antagonist, 1-850 is a thyroid hormone receptor antagonist, OPC-31260 is an antidiuretic hormone receptor antagonist, OPC-21268 is a vasopressin V1 receptor antagonist, FRBI is a FSH receptor-binding inhibitor, and αhCRH is a corticotropin releasing hormone receptor antagonist. As an alternative approach, EDN3-like polypeptides may be administered to a mutant mouse (such as a knock-out mouse, mouse with a partial loss-of-function mutation, or mouse expressing an RNAi construct) that has reduced activity of a particular receptor. The difference in RER between the mutant mouse and a wild-type mouse would indicate whether the mutant receptor is part of the EDN3-like pathway.

Similar methods may be used to identify the receptors that mediate EDN3-like anti-gluconeogenesis activity in hepatic cells. Example 8 discloses a cell culture assay for hepatocyte gluconeogenesis. EDN3 97-140 (or another EDN3-like polypeptide) can be added to hepatocyte cells in the presence or absence of a receptor antagonist. Any suitable receptor antagonist may be used, and a few examples are listed in the previous paragraph. One may also identify the receptor by contacting the cells with an EDN3-like polypeptide and a nucleic acid (such as an RNAi construct or siRNA) that reduces expression of a given receptor. Typically, the receptor antagonist or inhibitory nucleic acid is administered before the EDN3-like polypeptide. Alternatively, one may identify the receptor by testing the EDN3-like polypeptide on wild-type cells and cells that do not express the candidate receptor of interest.

Similar methods may be used to further characterize the receptor that mediates EDN3-like GLP-release activity. The CTX experiments in Example 5 indicate that the GLP-1-release activity of EDN3 97-140 is mediated by a G-protein coupled receptor. The G-protein coupled receptor may be further characterized using experiments such as the antisense inhibition, receptor mutation, or receptor antagonist experiments described above.

In addition to the cell-based or model organism-based assays above, the receptor for an EDN3-like polypeptide may be identified using a biochemical approach. For instance, the EDN3-like polypeptide may be used in an affinity purification scheme. The EDN3-like polypeptide may be anchored to a column or other substrate, and used to isolate binding partners such as receptors. Alternatively, to facilitate isolate of transmembrane receptors, the EDN3-like polypeptide can be allowed to bind receptors in a cellular context, crosslinked to a receptor, and then biochemically isolated. This isolation protocol may use an antibody specific to the EDN3-like polypeptide or by using a protein tag fused to the EDN3-like polypeptide. Alternatively, candidate receptors (or well-solubilizing fragments thereof) may be tested for binding to EDN3-like polypeptides in a cell-based or cell-free system.

Thus, in some embodiments the present disclosure provides a method of identifying an EDN3-like receptor, comprising: (a) contacting a test cell with an EDN3-like polypeptide and a receptor antagonist, (b) contacting a control cell with an EDN3-like polypeptide, and (c) determining the EDN3-like response of the test cell and control cell, wherein a greater EDN3-like response of the control cell compared to the test cell indicates that the receptor inhibited by the receptor antagonist is an EDN3-like receptor. This application also discloses a method of identifying an EDN3-like receptor, comprising: (a) contacting a test cell with an EDN3-like polypeptide, wherein the test cell comprises a mutation that reduces the activity of a receptor, (b) contacting a control cell with an EDN3-like polypeptide, wherein the control cell comprises wild-type activity of the receptor, and (c) determining the EDN3-like response of the test cell and control cell, wherein a greater EDN3-like response of the control cell compared to the test cell indicates that the receptor inhibited by the receptor antagonist is an EDN3-like receptor. The cells may be, for instance, hepatic cells, hypothalamic cells, or enteroendocrine cells. The EDN3-like polypeptide may be, for example, EDN3 97-140. The EDN3-like response may be, for example, GLP-1 release, a reduction in gluconeogenesis, or an increase in RER in a model organism. The receptor inhibitor may be a small molecule, a polypeptide, an antibody, or an antisense nucleic acid, for example. The mutation that reduces the activity of a receptor may be a null mutation, a partial loss of function mutation, a mutation in the coding region, or a mutation in the promoter, for example.

In certain embodiments, the EDN3-like polypeptide does not substantially activate endothelin-3 receptors. For instance, in certain embodiments, the EDN3-like polypeptide does not substantially bind to or activate the ET_{A} or ET_{B} endothelin receptor subtypes. In some embodiments, the EDN3-like polypeptide activates ET_{A} or ET_{B} to less than 50%, 20%, 10%, 5%, 2%, or 1% the activity achieved with Endothelin-3. In some embodiments, the EDN3-like polypeptide has substantially no activity in a rat aortic ring vasoconstriction assay. For example, the EDN3-like polypeptide has less than 50%, 20%, 10%, 5%, 2%, or 1% of the vasoconstriction activity that Endothelin-3 does.

### EXEMPLIFICATION

The invention now being generally described, it will be more readily understood by reference to the following examples, which are included merely for purposes of illustration of certain aspects and embodiments of the present invention, and are not intended to limit the invention. For example, the particular constructs and experimental design disclosed herein represent exemplary tools and methods for validating proper function. As such, it will be readily apparent that any of the disclosed specific constructs and experimental plan can be substituted within the scope of the present disclosure.

### INTRODUCTION

The hypothalamus is a rich source of peptide hormones involved in energy balance. Endothelin-3 was discovered in this brain region (Bloch, 1989). We have embarked on sequencing of a cDNA library generated from single neurons in the POA of the anterior hypothalamus, one of the major sites of regulation of core body temperature and energy metabolism (Eberwine and Bartfai, 2010). This sequencing, rather than microarray-based expression profiling, has provided a detailed molecular description of the transcriptome of individual neurons, with identification of rare mRNAs that can be missed through dilution in pooled mRNAs from several cells, or because the linear amplification often generates cDNAs biased towards the 3′ UTR sequence, while microarrays tend to have probes concentrated on the coding region. The cDNA encoding preproendothelin was identified in single preoptic area neurons. We have predicted a long form cleaved at GKR motif yielding a 44 amino acid, C-terminally amidated endothelin-like peptide. The predicted peptide (EDN3 97-140) was synthesized by solid phase synthesis and its effects in vivo upon hypothalamic (POA) energy expenditure and peripheral glucose metabolism were examined. In addition the effects of EDN3 97-140 ex vivo on isolated perfused rat colon and in vitro on several cell lines on glucagon-like peptide 1 (GLP-1) and gluconeogenesis were studied. We report that despite a common N terminal portion with endothelin this peptide does not act at ET_{A} or ET_{B} receptors nor mediate vasoconstriction in blood vessels. Further, EDN3 97-140 acts via a G protein-coupled receptor (GPCR) that is expressed in mouse neurons, murine GLUTag cells, rat colon and rat H4IIE hepatocytes. The EDN3 97-140 active peptide and its receptor represent novel targets for the treatment of metabolic disease and type 2 diabetes. Moreover, as detailed herein, the identification of this novel EDN3-like polypeptide having a novel activity profile has permitted and will continue to permit design and use of other EDN3-like polypeptides, such as variants of EDN3 97-140. The below examples focus primarily on analysis of the functional activity of the EDN3-like polypeptide referred to as EDN3 97-140. However, any of the particular variants described herein, either explicitly or prophetically, can also be assessed in these and other assays.

### Example 1

### EDN3 97-140, a 44 Amino Acid Peptide Identified from Mouse Endothelin-3

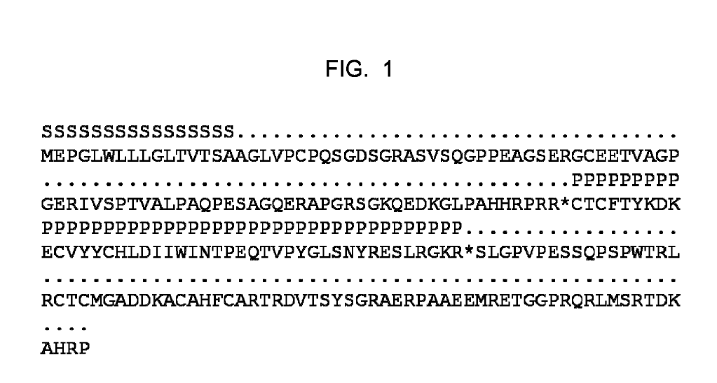

Gene expression data from warm-sensitive preoptic area (POA) neurons was used as the basis for the computational platform to generate a list of all genes found to be either expressed above the mean or have significant expression in at least one neuron (Eberwine and Bartfai, 2010). Each Gene Identifier from the expression data set was translated to the NCBI standard Gene Identifier (Entrez ID) and the corresponding protein was analyzed with the ProP1.0 proprotein cleavage site prediction to identify predicted cleavage products of less than 50 amino acids. Two propeptide cleavage sites of proendothelin-3 were identified (FIG. 1) resulting in a 44 amino acid peptide. The peptide was not represented as a defined product of proendothelin-3 in the Uniprot protein knowledge database (Apweiler, 2004) of annotated proteins and therefore deemed a novel ligand of interest to be characterized.

### Materials and Methods for Peptide Identification

The corresponding protein sequences of genes expressed above the mean from mouse POA neuron expression profiling was performed by matching the array probe data to a corresponding protein. The propeptide cleavage site detection algorithm Prop 1.0 (Duckert, 2004) was used to compute all sequences with signal peptides and di-basic cleavage sites. The results were manually curated on the basis of size, amino acid content, and predicted secondary structure. EDN3 97-140 was identified as a candidate from that list.

### Reagents

All chemicals and buffers were obtained from Sigma-Aldrich (St. Louis, Mo.), unless otherwise noted. EDN3 97-140 and related peptides were purchased from Bachem (King of Prussia, Pa.), CPC Scientific (San Jose, Calif.) or synthesized according to the following method. EDN3 97-140 was assembled on an Applied Biosystems ABI433 peptide synthesizer using a Rink amide MBHA resin (supplied by EMD Chemicals) and standard Fmoc amino acids (supplied by ABI). The synthesized resin was cleaved and deprotected by treatment of 0.5 g of synthesis resin with 10 mL of a TFA/water/phenol/thioanisole/ethanedithiol mixture (82.5:5:5:5:2.5) for 1 hour. The mixture was filtered, and the peptide was precipitated by dilution of the filtrate into 100 mL of diethyl ether. The crude peptide salt was collected, dried, and purified by reverse phase HPLC using a Waters XBridge C18 preparative column (19 mm×100 mm) at a flow rate of 17 mL/minute and a dual solvent gradient from 0 to 40% B over 40 minutes (solvent A, acetonitrile/water/trifluoroacetic acid (2:97.9:0.1); solvent B, 100% acetonitrile). Fractions were analyzed by LC-MS, and appropriate fractions were pooled to provide the linear peptide at approximately 95% purity. The combined fractions were lyophilized, and the dry peptide was stored at −20° C.

### Example 2

### EDN3 97-140 does not Bind to ET_{A}, ET_{B} or Cause Vasoconstriction in Rat Aorta Rings

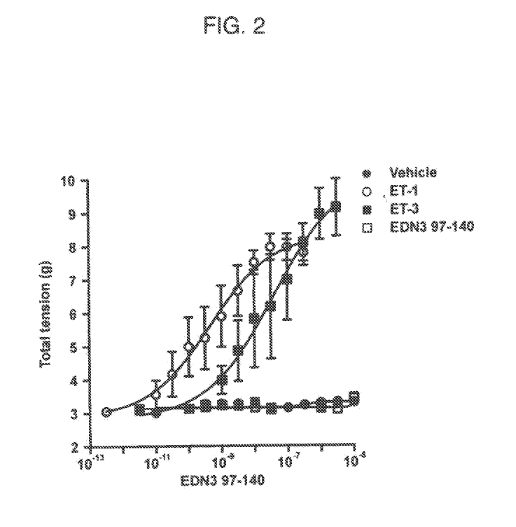

EDN3 97-140 failed to bind to either ET_{A} or ET_{B} receptors when tested at a concentration of up to 3 μM (p>0.05), whereas the positive controls endothelin-1 and endothelin-3 potently inhibited binding of the radiolabeled agent with IC₅₀'s of 70 pM and 16 pM, respectively. These results indicate that, in contrast to endothelin-1 and endothelin-3, EDN3 97-140 had no contractile effect even at the highest concentration of 10 μM (p>0.05; FIG. 2). These experiments indicate that EDN3 97-140 activity is distinct from that of Endothelin-1 and Endothelin-3.

### Materials and Methods for Determining ET_{A} and ET_{B} Binding In Vitro

To evaluate the affinity of EDN3 97-140 for the agonist site of the human endothelin ET_{A} receptor, radioligand binding with CHO cells transfected with the ET_{A} receptor was used. Cell membrane homogenates (10 μg protein) were incubated for 120 minutes at 37° C. with 0.03 nM [¹²⁵I]endothelin-1 in the absence or presence of the test compound in a buffer containing 50 mM Tris-HCl (pH 7.4), 5 mM MgCl₂ and 0.1% bovine serum albumin (BSA). Nonspecific binding was determined in the presence of 100 nM endothelin-1. Following incubation, the samples are filtered rapidly under vacuum through glass fiber filters (GF/B, Packard) presoaked with 0.1% BSA and rinsed several times with an ice-cold buffer containing 50 mM Tris-HCl and 150 mM NaCl using a 96-sample cell harvester (Unifilter, Packard). Filters were dried and then counted for radioactivity in a scintillation counter (Topcount, Packard) using a scintillation cocktail (Microscint 0, Packard). Results are expressed as a percent inhibition of the control radioligand specific binding. The standard reference compound was endothelin-1, which was tested in each experiment at several concentrations to obtain a competition curve from which the IC₅₀ was calculated.

To evaluate the affinity of EDN3 97-140 for the agonist site of the human endothelin ET_{B} receptor in transfected CHO cells, radioligand binding was used. Cell membrane homogenates (2.4 μg protein) are incubated for 120 minutes at 37° C. with 0.03 nM [¹²⁵1]endothelin-1 in the absence or presence of the test compound in a buffer containing 50 mM Tris-HCl (pH 7.4), 5 mM MgCl₂, 20 mg/L aprotinin and 0.1% BSA. Nonspecific binding was determined in the presence of 100 nM endothelin-1. Following incubation, the samples were filtered rapidly under vacuum through glass fiber filters (GF/B, Packard) presoaked with 0.1% BSA and rinsed several times with an ice-cold buffer containing 50 mM Tris-HCl and 150 mM NaCl using a 96-sample cell harvester (Unifilter, Packard). The filters were dried then counted for radioactivity in a scintillation counter (Topcount, Packard) using a scintillation cocktail (Microscint 0, Packard). Results were expressed as a percent inhibition of the control radioligand specific binding. The standard reference compound was endothelin-3, which was tested in each experiment at several concentrations to obtain a competition curve from which the IC₅₀ was calculated.

### Materials and Methods to Assay Rat Aorta Vasoconstriction Ex Vivo

The thoracic aorta with endothelium intact was isolated from male Wistar rats (300-500 g), cut into 3 mm rings, and placed in tissue baths with oxygenated Krebs-Henseleit buffer containing (in mM): KCl (4.7), CaCl₂-2H₂O (2.5), MgSO₄-7H₂O (1.2), KH₂PO₄ (1.2), D-glucose (10), NaHCO₃ (25), NaCl (118). Tissues were placed under a 3 g basal tension and allowed to equilibrate for 1 hour at 37° C. To confirm tissue viability, 40 mM KCl was applied for 15 minutes followed by 10 μM carbachol for 3 minutes and then allowed to wash for 1 hour prior to compound treatment. Cumulative concentration-response curves were performed using vehicle (MilliQ), endothelin-1 (10 pM-300 nM), endothelin-3 (100 pM-3 μM) or EDN3 97-140 (100 pM-10 μM), with each concentration being applied for at least 15 minutes to ensure contractile responses had plateaued.

### Example 3

### EDN3 97-140 Peptide Causes Hyperthermia and Reduces the Respiratory Exchange Ratio (RER) in Mice

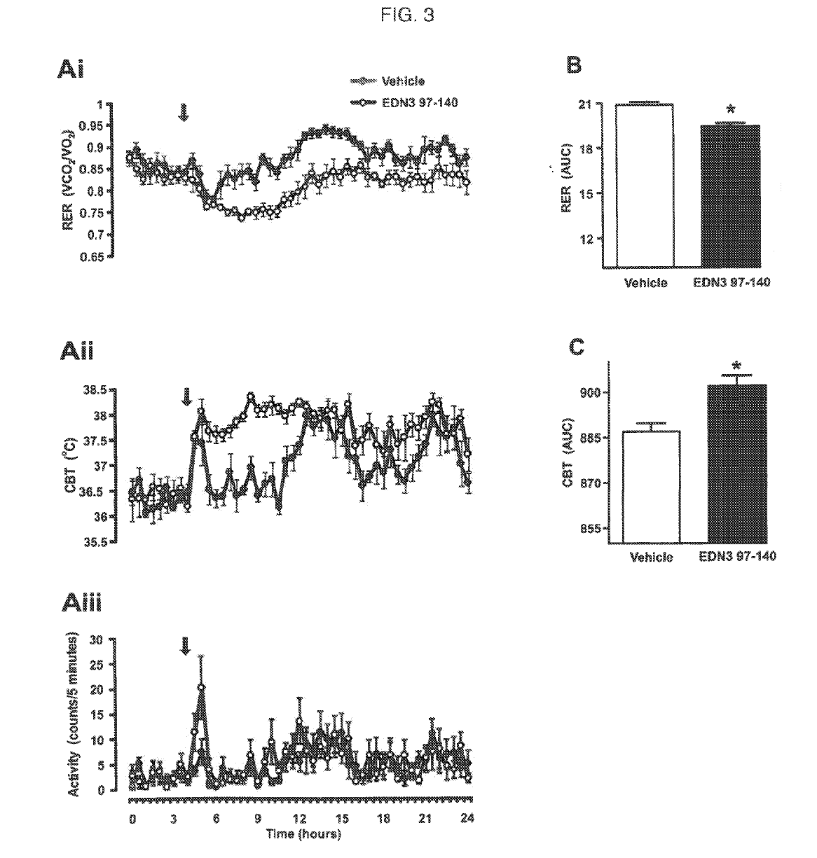

Consistent with its identification in warm sensitive neurons, EDN3 97-140 affects body temperature in a mouse model. Changes in RER were measured by indirect calorimetry (VCO₂/VO₂). The area under the curve (AUC) of RER and the core body temperature measurements were treated as two primary endpoints of interest to assess the effect of EDN3 97-140 (FIG. 3). The AUC of locomotor activity was treated as secondary endpoint. In lean mice, 2.5 nmol EDN3 97-140 injected into the POA elicited a sustained decrease in the AUC of RER compared to vehicle treated mice (FIG. 3Ai & 3B). The decrease in RER indicates a switch from glucose metabolism to elevated fatty acid utilization. In addition, EDN3 97-140 caused hyperthermia compared to vehicle (FIG. 3Aii & 3C). A transient increase in locomotor activity was observed with EDN3 97-140 injection in the POA (FIG. 3Aiii). Locomotor activity did not solely contribute to the CBT increase because this effect of EDN3 97-140 was transient, whereas sustained effects on RER and CBT were observed post the transient locomotor effect. Thus, EDN3 97-140 administered to the POA increases body temperature, and this effect is not explained solely by locomotor activity.

### Materials and Methods for Indirect Calorimetry in Mice

C57BL6J male mice (3-4 months old, 25-30 g, 6 mice per group) were purchased from Harlan. Standard diet animals were fed ad libitum with mouse breeder diet (S-2335 Mouse Breeder, 17.50% protein, 11.72% fat, 3.36% fiber, energy 3.52 kcal/g; Harlan Teklad (Madison, Wis.). For telemetry and metabolic studies, male mice were surgically implanted with radiotelemetry devices (TA-F20, Data Sciences, St. Paul, Minn.) into the peritoneal cavity for CBT and locomotor activity evaluation. Indirect calorimetry and telemetry were performed simultaneously in standard diet-fed mice housed in individual acclimated, clear respiratory chambers (20×10×12.5 cm), using a computer-controlled, open-circuit system (Oxymax System) that is part of an integrated Comprehensive Lab Animal Monitoring System (CLAMS; Columbus Instruments, Columbus, Ohio). Air was passed through chambers at a flow rate of ˜0.5 L/minute. Exhaust air from each chamber was sampled at 30 minute intervals for 1 minute. Sample air was sequentially passed through O₂ and CO₂ sensors (Columbus Instruments) for determination of O₂ and CO₂ content, from which measures of oxygen consumption (VO₂) and carbon dioxide production (VCO₂) were estimated. Outdoor air reference values were sampled after every 4 measurements. Gas sensors were calibrated prior to the onset of experiments with primary gas standards containing known concentrations of O₂, CO₂, and N₂ (Airgas Puritan Medical, Ontario, Calif.). Energy expenditure measures (VO₂, VCO₂ and heat formation ((3.815+1.232*RER)*VO₂ (in liters)) were corrected for estimated effective metabolic mass per Kleiber's power function (Kleiber, 1947). Mice undergoing indirect calorimetry were acclimated to the respiratory chambers for 3-4 days before the onset of study. Data were recorded under ambient room temperature clamped at 25° C., beginning from the onset of the light cycle (12:12 hour light-dark cycle; lights on at 6:00 a.m.) for 3 days. Treatments were administered directly to the POA via an implanted cannula (anterior from bregma: 0.3 mm, midline, ventral: 3.8 mm, cannula 26 GA, 10 mm length) using an injector (33 GA, protruding 0.4 mm beyond the tip of the cannula, total length 10.4 mm) connected to plastic tubing and a microsyringe (10 μL) in a volume of 0.5 μL over a period of 5 minutes to allow diffusion. Each mouse received both vehicle and EDN3 97-140 in consecutive experiments. Area under the curve (AUC) was used for measuring the effect over the time course of study. Statistical analysis was performed by one sided t-test adjusted by Holm procedure.

### Example 4

### EDN3 97-140 Improves Glucose Tolerance in Diabetic Mice

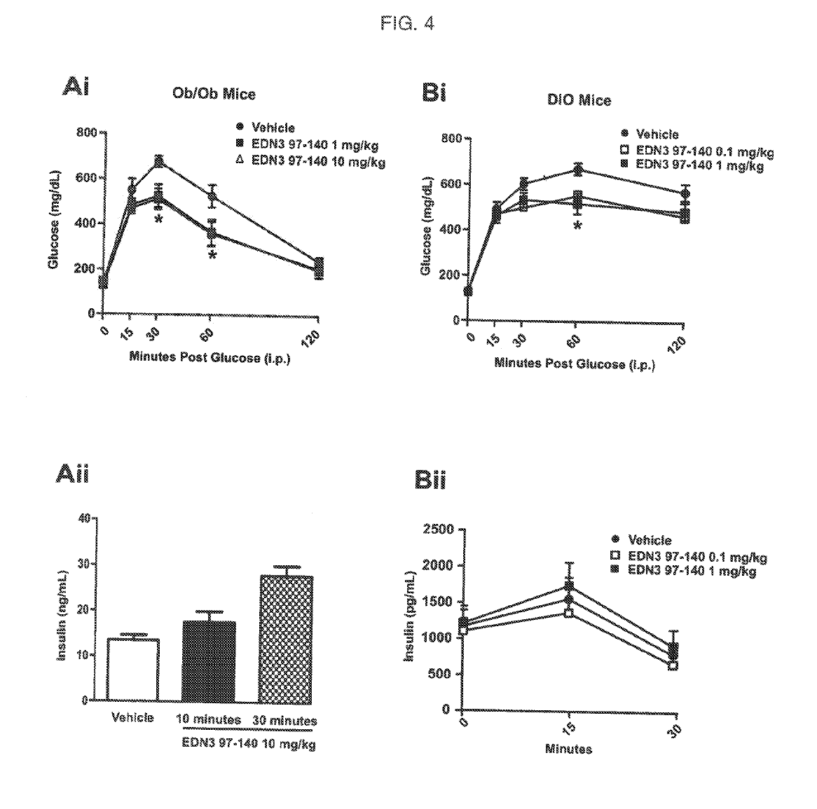

Reports proposing that the hypothalamus is a major attributor involved in the central control of glucose homeostasis (see Zsombok and Smith, 2009) led us to investigate EDN3 97-140 peripheral activities related to glucose homeostasis. Acute peripheral administration of EDN3 97-140 (0.1, 1 or 10 mg/kg; i.p.) decreased the glucose excursion curve in response to an IPGTT with no statistically significant effect on insulin in both ob/ob and diet induced obese (D10) mice (FIG. 4). The lack of a statistically significant change in insulin levels suggests EDN3 97-140 may be a potential insulin sensitizer. There was no effect of EDN3 97-140 (1 and 10 mg/kg i.p.) with acute administration on an insulin tolerance test (data not shown). Thus, EDN3 97-140 may act through the hypothalamus to promote insulin sensitivity.

### Materials and Methods to Assay Glucose Tolerance in Diabetic Mice

DIO mice fed on a high fat diet for 14 weeks and ob/ob mice were purchased from Jackson Labs. Ob/Ob mice and DIO mice used in all studies were used at 7 and 18 weeks old, respectively. After an overnight fast, ob/ob and DIO mice were injected with vehicle (20 mM Na acetate/140 mM NaCl) or EDN3 97-140 (0.1, 1 or 10 mg/kg i.p.) followed by a 0.6 mg/Kg (i.p.) or 2 g/kg (i.p.) D-glucose challenge in ob/ob and DIO mice respectively. Plasma glucose levels were determined from mouse tail vein bleeds at 0, 15, 30, 60, and 120 minutes after compound injection and measured using an automatic glucometer (Alpha-Trak). To determine the acute insulin release, plasma insulin levels were measured by using an ELISA kit with mouse insulin as a standard (Meso Scale Discovery). Results were expressed as percentages of vehicle concentrations and statistical significance determined by 2-way ANOVA repeated measures.

### Example 5

### EDN3 97-140 Stimulates GLP-1 Secretion from GLUTag Cells Via a Gₛ-Dependent Mechanism

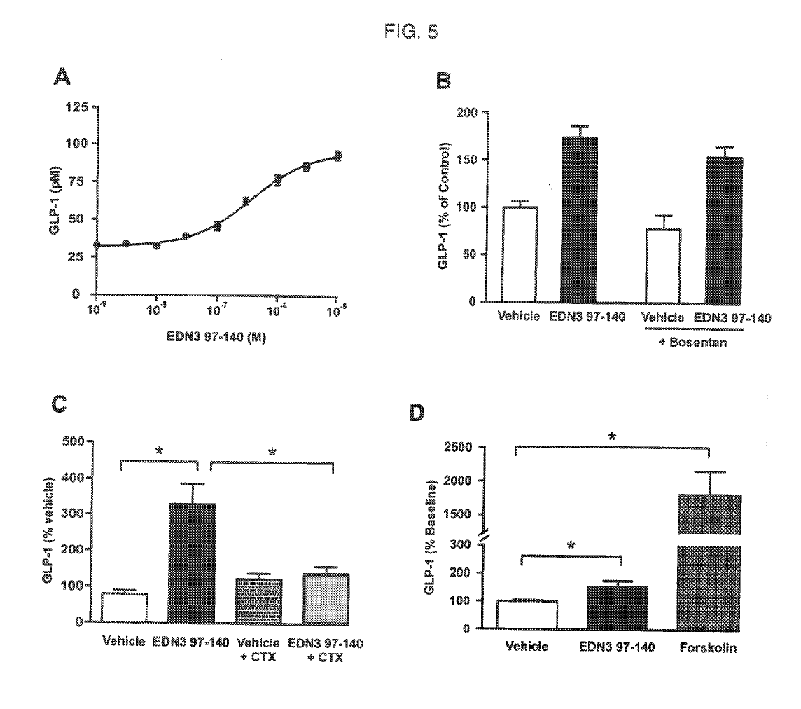

In an effort to understand the potential role of EDN3 97-140 in regulating metabolism, EDN3 97-140 was tested in several cell based assays. EDN3 97-140 (10 μM) had no effect on 3T3L1 adipocyte basal glucose uptake and also failed to modulate glucose-stimulated insulin release from INS1 cells (data not shown). In contrast, EDN3 97-140 stimulated GLP-1 secretion from GLUTag enteroendocrine cells in a concentration-dependent manner with an EC₅₀ of 369±68 nM (FIG. 5A). Endothelin-3 was also tested and had no effect on GLUTag GLP-1 secretion at the highest concentration (10 μM; Table 2), confirming that EDN3 97-140 has an activity that is distinct from Endothelin-3. Similarly, the EDN3 97-140 stimulated GLP-1 secretion was not inhibited with a high concentration of the non subtype-selective potent ET_{A}/ET_{B} antagonist bosentan (FIG. 5B). This result confirms that EDN3 97-140 acts through a receptor other than ET_{A}/ET_{B}. However, the EDN3 97-140 (1 μM) stimulated GLP-1 secretion was blocked by overnight pre-treatment with CTX (FIG. 5C). Because CTX is a specific inhibitor of Gαₛ, EDN3 97-140 may act through a Gαₛ-associated receptor. In conclusion, the effects of EDN3 97-140 are not confined to the hypothalamus. Rather, EDN3 97-140 can directly promote GLP-1 secretion from enteroendocrine cells. The experiments thus suggest that EDN3 97-140 acts through two distinct mechanisms.

GLP-1 is an intestinal hormone that is secreted during meal absorption and is essential for normal glucose homeostasis. GLP is believed to act directly on pancreatic beta cells to increase insulin secretion. However, the relatively low plasma levels and rapid metabolism of GLP-1, raise questions as to whether direct endocrine action on target organs, such as islet cells, account for all of its effects on glucose tolerance. Several studies suggest glucose homeostasis via an indirect pathway on insulin secretion, glucose production and glucose utilisation is mediated via the hepatoportal sensor vagal afferent pathway (Nakabayashi, 1996; Balkan, 2000; Burcelin, 2001; Nishizawa, 2003; Dardevet, 2004; Ahren, 2004; Ionut, 2005; Vahl, 2007). The increase in GLP-1 evoked by EDN3 97-140 therefore may increase insulin secretion directly in pancreatic beta cells or may improve insulin sensitivity via the hepatoportal vagal afferent circuitry. Without wishing to be bound by theory, EDN3 97-140 may exert its anti-diabetic effects by increasing insulin secretion or improving insulin sensitivity.

TABLE 2
EDN3 97-140 structure activity analysis of 15 truncated or variant
EDN3-like polypeptides for activity in GLP-1 release GLUTag assays. This analysis is
exemplary of the making and testing of variants of EDN3 97-140 to identify those
variants that can be used as EDN3-like polypeptides (e.g., those variants that maintain,
for example, the activity of EDN3 97-140. The current analysis suggests a role for
protein folding and intact termini for full activity.

| | | SEQ | |
|---|---|---|---|
| Name | Sequence | ID | EC₅₀ |
| EDN3 97- | CTCFTYKDKECVYYCHLDIIWINTPEQTVPYGLS | 21 | 369 nM |
| 140 | NYRESLRGKR-NH₂ | | |
| EDN3 97- | CTCFTYKDKECVYYCHLDIIWINTPEQTVPYGLS | 29 | 1721 nM |
| 137 | NYRESLR-NH₂ | | |
| EDN3 98- | TCFTYKDKECVYYCHLDIIWINTPEQTVPYGLSN | 34 | >10 uM |
| 140 | YRESLRGKR-NH₂ | | |
| EDN3 | FTYKDKECVYYCHLDIIWINTPEQTVPYGLSNY | 35 | >10 uM |
| 100-140 | RESLRGKR-NH₂ | | |
| EDN3 | FTYKDKESVYY-NH₂ | 36 | >10 uM |
| 100-110 | | | |
| EDN3 | YYSHLDIIWINTPEQTVPYGLSNYRESLRGKR- | 37 | >10 uM |
| 109-140 | NH₂ | | |
| C111S* | | | |
| EDN3 | YYAHLDIIAINTPEQTVPYGLSNYRESLRGKR- | 38 | >10 uM |
| 109-140 | NH₂ | | |
| C111A | | | |
| W117A* | | | |
| EDN3 | INTPEQTVPYGLSNYRESLRGKR- | 39 | >10 uM |
| 118-140 | NH₂ | | |
| EDN3 97- | CTCFTYKDKECVYYCHLDIIW-OH | 40 | >10 uM |
| 117, | | | |
| endothelin- | | | |
| 3 | | | |
| EDN3 | YKDKECVYYCHLDIIWINTPEQ-NH₂ | 41 | >10 uM |
| 102-123 | | | |
| EDN3 97- | STSFTYKDKESVYYSHLDIIWINTPEQTVPYGLS | 42 | >10 uM |
| 140 C->S | NYRESLRGKR-NH₂ | | |
| mutant | | | |
| EDN3 97- | CTCFTYKDKECVYYCHLDIIWINTPEQTVPYGLS | 21 | 2 uM |
| 140 free | NYRESLRGKR-OH | | |
| acid | | | |
| EDN3 97- | CTCFTYKDKECVYYCHLDIIHINTPEQTVPYGLS | 43 | >1 uM |
| 140 | NYRESLRGKR-NH₂ | | |
| W117H | | | |
| EDN3 97- | CTCFTYKDKECVYYCHLDIIFINTPEQTVPYGLS | 30 | 731 nM |
| 140 | NYRESLRGKR-NH₂ | | |
| W117F | | | |
| EDN3 97- | ATCFTYKDKECVYYAHLDIIWINTPEQTVPYGLS | 31 | 2.7 uM |
| 140 C97A, | NYRESLRGKR-NH₂ | | |
| C111A | | | |
| EDN3 97- | CTAFTYKDKEAVYYCHLDIIWINTPEQTVPYGLS | 32 | 1.6 uM |
| 140 C99A, | NYRESLRGKR-NH₂ | | |
| C107A | | | |

*The polypeptides EDN3 109-140 C111S and EDN3 109-140 C111A W117A showed approximately 20% of maximal activity when delivered at the highest concentration (10 μM). Because the activity did not reach 50% of the maximum, the EC₅₀ is listed as “>10 μM” in Table 2.

### Example 6

### Structure-Function Analysis of EDN3 97-140-Mediated GLP-1 Release Activity in GLUTag Cells

EDN3 97-140 activities were explored using a series of 15 truncated peptides tested for GLP-1 release activity in GLUTag cells (Table 2). Endothelin-3 (EDN3 97-117) was not sufficient to elicit a GLP-1 release. In contrast, the 44-mer C-terminally amidated EDN3 97-140 was the most potent peptide tested.

Endothelin-3 has four cysteine residues. The cysteines, located at positions 1, 3, 11, and 15, may form two disulfide bonds as reported for endothelin-1 (Janes, 1994). Therefore, cysteine substitution mutants were synthesized to determine whether the formation of both disulfide bridges contributes to folding and agonist activity. EDN3 98-140 (lacking the first cysteine) and EDN3 100-140 (lacking the first 2 cysteines) do not measurably stimulate GLP-1 secretion from GLUTag cells. EDN3 97-140 cysteine mutants which eliminate the disulfide bonds one at a time, reduce the potency of EDN3 97-140; however, activity is still observed. Specifically, EDN3 97-140 C97A, C111A had an EC₅₀ value of 2.7 μM and EDN3 97-140 C99A, C107A had an EC₅₀ of 1.6 μM.

The structure activity relationships reported in Table 2 suggests endothelin-3 treatment of GLUTag cells in vitro does not result in GLP-1 secretion, clearly differentiating EDN3 97-140 from endothelin-3; a functional differentiation also confirmed in both the vasoconstriction and ET_{A}/ET_{B} receptor binding assays. The experiments with truncations and point mutants of EDN3 97-140 indicate that N and C-termini and certain cysteine residues help promote GLP-1 release from GLUTag cells. Based on the endothelin-1 crystal structure (Janes, 1994), we predict that cysteine 97 and cysteine 111 form a disulfide bond as well as cysteine 99 and cysteine 107. The 2 cysteine bridges may assist the folding of the N-terminus. The partial loss of activity seen with EDN3 97-137 (which lacks the C-terminal GKR) suggests that both ends of the peptide contribute to its activity. This contribution may be due, for example, to length, folding, or charge, and thus, the ends of an active peptide variant may tolerate amino acid substitution. For example, in other portions of the molecule, a conservative substitution such as EDN3 97-140 W117F does not cause a loss of GLP-1 secretagogue function in vitro and demonstrates the potential for generating further EDN3-like polypeptides by making variants of the polypeptides provided herein.

### Materials and Methods for Cell Culture

Cell culture reagents were purchased from Invitrogen (Gathersburg, Md.) unless otherwise noted. GLUTag cells were obtained from Dr. Daniel Drucker and cultured in High Glucose DMEM medium supplemented with 100 U/mL penicillin, 100 μg/mL streptomycin, and 10% fetal calf serum at 10% CO₂ at 37° C. H4IIE rat hepatoma cells were purchase from American Type Culture Collection (Manassas, Va.) and cultured in low glucose DMEM supplemented with 100 U/mL penicillin, 100 μg/mL streptomycin, 2 mM L-glutamine and 10% heat-inactivated fetal calf serum at 5% CO₂ and 37° C.

Materials and Methods to Determine GLP-1 Secretion from GLUTag Cells In Vitro

GLUTag cells were plated into Poly-D-Lysine coated 96-well plates and treated according to the protocol established by Brubaker, 1998. Briefly, cells were washed twice with DMEM 5 mM glucose and then starved in DMEM containing 5 mM glucose for 2 hours. Cells were stimulated with DMEM containing 15 mM glucose with peptide or dimethyl sulfoxide (DMSO). The EC₅₀ concentration was calculated using GraphPad Prism software (GraphPad, La Jolla, Calif.). GLP-1 was measured immediately or frozen for later measurement using an ELISA for active GLP-1(7-36) amide according to the manufacturer protocol and quantity was determined by assay standard curves (Millipore). For studies with cholera toxin (CTX), cells were pretreated with CTX (0.2 μg/mL) overnight. CTX was also present during serum/glucose starvation and incubation with 1 μM EDN3 97-140 or DMSO vehicle. Data were calculated as percent vehicle to normalize across multiple experiments. Standard error for the estimate of EC₅₀ was approximated by the Delta method and significance determined by one sided Dunnett using a two sample unpaired t-test. Statistical analysis of CTX data was performed using a two sample unequal variance t-test (Satterthwaite's test). The Holm testing procedure was applied to account for the multiplicity issue due to multiple comparisons.

### Example 7

### EDN3 97-140 Stimulates GLP-1 Secretion within the Rat Perfused Colon

To confirm translation of EDN3 97-140 from mouse GLUTag cells in vitro to stimulated GLP-1 release in the portal vein, the rat perfused colon preparation ex vivo was used. EDN3 97-140 (200 nM) perfused via the superior mesenteric artery stimulated GLP-1 secretion by 56±18% which is consistent with a 10 pM increase in GLP-1 release (FIG. 5D). The rat perfused colon assay thus confirms the observation in Example 6 that EDN3 97-140 promotes GLP-1 secretion.

Materials and Methods for Assaying GLP-1 Secretion from Rat Perfused Colon Ex Vivo

Ex vivo rat colon vascular perfusion experiments were performed as previously described (Plaisancié, 1995). Male Sprague-Dawley rats (300 g) were purchased from Charles River and fed ad libitum with Purina Lab Chow #5001 (WF Fisher and Son Inc Sommerville, N.J.). Rats were decapitated and the abdomen was opened with a midline incision. The superior mesenteric artery and portal vein were quickly cannulated by a metal cannula and plastic tubing, respectively. The arterial vascular perfusion started immediately with an oxygenated Krebs-Henseleit buffer containing-solution at a rate of 2 mL/minute [solution: Krebs-Henseleit buffer (25.0 mM NaHCO₃; 118 mM NaCl; 4.7 mM KCl; 1.2 mM MgSO₄; 1.2 mM KH₂PO₄; 2.5 mM CaCl₂) with 3% BSA, 5 mM glucose, MEM essential and nonessential amino acids (pH 7.4)]. The colonal lumen was perfused at the rate of 0.5 mL/minute. The remaining colon and small intestine were removed after the ligation of their respective supplying vessels. After the colon preparation was transferred to a 37° C. tissue bath, the portal effluent was collected as 5 minute fractions. The fraction aliquots were frozen at −20° C. for subsequent determinations of GLP-1. EDN3 97-140 was dissolved at 200 nM in vascular perfusion solution and perfused into the artery after a 30 minute baseline collection. At the end of the experiment, forskolin (10 μM) was perfused to serve as a positive control. The GLP-1 detection assay was carried out by using a commercial kit for active GLP-1 (7-36) amide according to the manufacturer's instructions (Millipore). GLP-1 release data was normalized to % baseline using the average of fractions collected prior to treatment as the baseline value and the peak data following each treatment. The average data across 3 independent experiments was reported comparing EDN3 97-140 versus control (baseline) and forskolin versus control (baseline). Statistical analysis was performed by one sided Satterwaite's test due to unequal variance and the Holm test was applied to account for multiple comparisons.

### Example 8

### EDN3 97-140 Inhibits Hepatocyte Gluconeogenesis

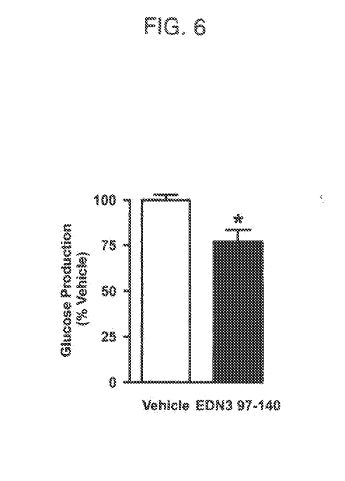

To assess the role of EDN3 97-140 on gluconeogenesis, the rat hepatoma cell line H4IIE was used. H4IIE cells were starved, supplemented with lactate and pyruvate as carbon sources, and were treated with 100 nM EDN3 97-140 for 24 hours. Basal de novo glucose production was reduced 19.5±7.6% by EDN3 97-140 versus vehicle treatment (FIG. 6).

EDN3 97-140 suppressed basal gluconeogenesis in the rat hepatocyte H4IIE cell line, an effect that was independent of insulin. As elevated basal glucose production by liver gluconeogenesis is a well known consequence of type 2 diabetes (see Rizza, 2010), EDN3 97-140 has the potential to modulate glucose homeostasis directly at the level of the liver to modulate hyperglycemia. This is consistent with the conclusion that EDN3 97-140 has activity to promote healthy glucose metabolism by lowering the amount of glucose produced in the liver.

Because EDN3 97-140 has secretagogue activity in mouse GLUTag cells and in the rat perfused colon causing GLP-1 secretion, and it suppresses gluconeogenesis directly on rat hepatoma cells, EDN3 97-140 may be a hormone with dual anti-hyperglycemic mechanisms.

### Materials and Methods to Assay H4IIE Glucose Production In Vitro

H4IIE cells were plated at 120,000 cells per well into 96-well Poly-D-Lysine coated plates and treated using a modification of the protocol as previously reported (de Raemy-Schenk, 2006). Briefly, 4 hours after plating, growth medium was exchanged for glucose production medium (glucose-free DMEM, 2 mM sodium pyruvate, and 20 mM sodium lactate) for 18 hours and then cells were treated with EDN3 97-140 or vehicle in fresh glucose production medium. After 24 hours, media was collected and assayed for glucose using an Amplex Red Glucose/Glucose Oxidase Assay Kit purchased from Invitrogen (Gathersburg, Md.). Data was reported as a percent of vehicle to normalize across experiments. A mixed effect model was used to analyze the H4IIE glucose production with the treatment group as a fixed effect and date as a random effect, and one sided hypothesis testing was conducted.

### BIBLIOGRAPHY

1. Hu R M, Han Z G, Song H D, et al. Gene expression profiling in the human hypothalamus-pituitary-adrenal axis and full-length cDNA cloning. PNAS 2000; 97(17):9543-9548.
2. Bloch K D, Eddy R L, Shows T B, Quertermous T. Cloning and chromosomal assignment of the gene encoding endothelin 3. J Biol Chem 1989; 264:18156-18161.
3. Eberwine J, Bartfai T. Single cell transcriptomics of hypothalamic warm sensitive neurons that control core body temperature and fever response. Signaling asymmetry and an extension of chemical neuroanatomy. Pharmaracol Ther 2010; ahead of print
4. Duckert P, Brunak S, Blom N. Prediction of proprotein convertase cleavage sites. Protein Eng Sel 2004; 17(1):107-12.
5. Kleiber M. “Body size and metabolic rate”. Physiol Rev 1947; 27:511-541.
6. Plaisancié P, Bernard C, Chayviallea J A, Cuber J C. Release of Peptide Y Y by neurotransmitters and gut hormones in the isolated, vascularly perfused rat colon. Scand J Gastroenterol 1994; 30:568-574.
7. de Raemy-Schenk A M, Trouble S, Gaillard P, Page P, Gotteland J P, Scheer A, Lang P, Yeow K. A cellular assay for measuring the modulation of glucose production in H4IIE cells. Assay Drug Dev Technol 2006; 4(5):525-533.
8. Apweiler R, Bairoch A, Wu C H. Protein sequence databases. Curr Opin Chem Biol 2004; 8(1):76-80.
9. Holst J J, The Physiology of Glucagon-like Peptide 1. Physiol Rev 1997; 87:1409-1439.
10. Beinfield M C. Biosynthesis and processing of pro CCK: recent progress and future challenges. Life Sci 2003; 72(7):747-757.
11. Inoue A, Yanagisawa M, Takuwa Y, Mitsui Y, Kobayashi M, Masaki T. The human preproendothelin-1 gene. Complete nucleotide sequence and regulation of expression. J Biol Chem 1989; 264(25):14954-14959.
12. Janes R W, Peapus D H, Wallace B A. The crystal structure of human endothelin. Nat Structure Bio 1994; 1:311-319.
13. Schweizer A, Valdenaire O, Nelböck P, Deuschle U, Dumas Milne Edwards J B, Stumpf J G, Löffler B M. Human endothelin-converting enzyme (ECE-1): three isoforms with distinct subcellular localizations. Biochem J 1997; 328:871-877.
14. Zsombok A, Smith B N. Plasticity of central autonomic neural circuits in diabetes. Biochim et Biophys Acta 2009; 1792:423-431.
15. Rizza R A. Pathogenesis of Fasting and Postprandial Hyperglycemia in Type 2 Diabetes: Implications for Therapy. Diabetes 2010; 59(11):2697-2707.
16. Nakabayashi H, Nishizawa M, Nakagawa A, Takeda R, Niijima A. Vagal hepatopancreatic reflex effect evoked by intraportal appearance of tGLP-1. Am J Physiol 1996; 271(5 Pt 1):E808-813.
17. Balkan B, Li X. Portal GLP-1 administration in rats augments the insulin response to glucose via neuronal mechanisms. Am J Physiol 2000; 279:R1449-R1454.
18. Burcelin R, Da Costa A, Drucker D, Thorens B: Glucose competence of the hepatoportal vein sensor requires the presence of an activated glucagon-like peptide-1 receptor. Diabetes 2001, 50:1720-1728.
19. Nishizawa M, Moore M C, Shiota M, Gustayson S M, Snead W L, Neal D W, Chemington A D. Effect of intraportal glucagon-like peptide-1 on glucose metabolism in conscious dogs. Am J Physiol 2003; 284:E1027-E1036.
20. Dardevet D, Moore M C, Neal D, DiCostanzo C A, Snead W, Chemington A D. Insulin-independent effects of GLP-1 on canine liver glucose metabolism: duration of infusion and involvement of hepatoportal region. Am J Physiol Endocrinol Metab 2004; 287:E75-81.
21. Ahren B. Sensory nerves contribute to insulin secretion by glucagon-like peptide-1 in mice. Am J Physiol 2004; 286:R269-R272.
22. Ionut V, Hucking K, Liberty I F, Bergman R N. Synergistic effect of portal glucose and glucagon-like peptide-1 to lower systemic glucose and stimulate counter-regulatory hormones. Diabetologia 2005; 48:967-975.
23. Vahl T P, Tauchi M, Durler T S, Elfers E E, Fernandes T M, Bitner R D, Ellis K S, Woods S C, Seeley R J, Herman J P et al. Glucagon-like peptide-1 (GLP-1) receptors expressed on nerve terminals in the portal vein mediate the effects of endogenous GLP-1 on glucose. Endocrinology 2007; 148(10):4965-73.

SEQUENCE LISTING

SEQ ID No. 1
EDN3 97-136
CTCFTYKDKECVYYCHLDIIWINTPEQTVPYGLSNYRESL
SEQ ID No. 2
EDN3 97-136 consensus
X₁TX₂FTYKDKEX₃VYYX₄HLDIIX₅INTPEQTVPYGLSNYRX₆SX₇
SEQ ID No. 3
EDN3 109-123 C111S
YYSHLDIIWINTPEQ
SEQ ID No. 4
EDN3 109-123 C111A W117A
YYAHLDIIAINTPEQ
SEQ ID No. 5
EDN3 109-137 C111S consensus
YYSHLDIIWINTPEQTVPYGLSNYRX₆SX₇R
SEQ ID No. 6
EDN3 109-137 C111A W117A consensus
YYAHLDIIAINTPEQTVPYGLSNYRX₆SX₇R
SEQ ID No. 7
EDN3 109-123
YYCHLDIIWINTPEQ
SEQ ID No. 8
EDN3 109-123 consensus
YYX₄HLDIIX₅INTPEQ
SEQ ID No. 9
EDN3 97-123
CTCFTYKDKECVYYCHLDIIWINTPEQ
SEQ ID No. 10
EDN3 97-123 consensus
X₁TX₂FTYKDKEX₃VYYX₄HLDIIX₅INTPEQ
SEQ ID No. 11
EDN3 97-127
CTCFTYKDKECVYYCHLDIIWINTPEQTVPY
SEQ ID No. 12
EDN3 97-127 consensus
X₁TX₂FTYKDKEX₃VYYX₄HLDIIX₅INTPEQTVPY
SEQ ID No. 13
hEDN3 97-137
CTCFTYKDKECVYYCHLDIIWINTPEQTVPYGLSNYRGSFR
SEQ ID No. 14
EDN3 97-137
X₁TX₂FTYKDKEX₃VYYX₄HLDIIX₅INTPEQTVPYGLSNYRX₆SX₇R
SEQ ID No. 15
EDN3 109-137
YYCHLDIIWINTPEQTVPYGLSNYRGSFR
SEQ ID No. 16
EDN3 109-137 consensus
YYX₄HLDIIX₅INTPEQTVPYGLSNYRX₆SX₇R
SEQ ID No. 17
EDN3 109-140
YYCHLDIIWINTPEQTVPYGLSNYRGSFRGKR
SEQ ID No. 18
EDN3 109-140 consensus
YYX₄HLDIIX₅INTPEQTVPYGLSNYRX₆SX₇RGKR
SEQ ID No. 19
hEDN3 97-140
CTCFTYKDKECVYYCHLDIIWINTPEQTVPYGLSNYRGSFRGKR
SEQ ID No. 20
EDN3 97-140 consensus
X₁TX₂FTYKDKEX₃VYYX₄HLDIIX₅INTPEQTVPYGLSNYRX₆SX₇RGKR
SEQ ID No. 21
EDN3 97-140
CTCFTYKDKECVYYCHLDIIWINTPEQTVPYGLSNYRESLRGKR
SEQ ID No. 22
EDN3 109-140 C111S consensus
YYSHLDIIWINTPEQTVPYGLSNYRX₆SX₇RGKR
SEQ ID No. 23
EDN3 109-140 C111S W117A consensus
YYAHLDIIAINTPEQTVPYGLSNYRX₆SX₇RGKR
SEQ ID No. 24
EDN3 102-123
YKDKECVYYCHLDIIWINTPEQ
SEQ ID No. 25
EDN3 102-123 consensus
YKDKEX₃VYYX₄HLDIIX₅INTPEQ
SEQ ID No. 26
EDN3 97-135
CTCFTYKDKECVYYCHLDIIWINTPEQTVPYGLSNYRES
SEQ ID No. 27
EDN3 97-135 consensus
X₁TX₂FTYKDKEX₃VYYX₄HLDIIX₅INTPEQTVPYGLSNYRX₆S
SEQ ID No. 28
hEDN3 97-135
CTCFTYKDKECVYYCHLDIIWINTPEQTVPYGLSNYRGS
SEQ ID No. 29
EDN3 97-137
CTCFTYKDKECVYYCHLDIIWINTPEQTVPYGLSNYRESLR
SEQ ID No. 30
EDN3 97-140 W117F
CTCFTYKDKECVYYCHLDIIFINTPEQTVPYGLSNYRESLRGKR
SEQ ID No. 31
EDN3 97-140 C97A, C111A
ATCFTYKDKECVYYAHLDIIWINTPEQTVPYGLSNYRESLRGKR
SEQ ID No. 32
EDN3 97-140 C99A, C107A
CTAFTYKDKEAVYYCHLDIIWINTPEQTVPYGLSNYRESLRGKR
SEQ ID No. 33
EDN3 97-124 consensus
X₁TX₂FTYKDKEX₃VYYX₄HLDIIX₅INTPEQT
SEQ ID No. 34
EDN3 98-140
TCFTYKDKECVYYCHLDIIWINTPEQTVPYGLSNYRESLRGKR
SEQ ID No. 35
EDN3 100-140
FTYKDKECVYYCHLDIIWINTPEQTVPYGLSNYRESLRGKR
SEQ ID No. 36
EDN3 100-110
FTYKDKESVYY
SEQ ID No. 37
EDN3 109-140 C111S
YYSHLDIIWINTPEQTVPYGLSNYRESLRGKR
SEQ ID No. 38
EDN3 109-140 C111A W117A
YYAHLDIIAINTPEQTVPYGLSNYRESLRGKR
SEQ ID No. 39
EDN3 118-140
INTPEQTVPYGLSNYRESLRGKR
SEQ ID No. 40
Endothelin-3 (EDN3 97-117)
CTCFTYKDKECVYYCHLDIIW
SEQ ID No. 41
EDN3 102-123
YKDKECVYYCHLDIIWINTPEQ
SEQ ID No. 42
EDN3 97-140 C->S mutant
STSFTYKDKESVYYSHLDIIWINTPEQTVPYGLSNYRESLRGKR
SEQ ID No. 43
EDN3 97-140 W117H
CTCFTYKDKECVYYCHLDIIHINTPEQTVPYGLSNYRESLRGKR
SEQ ID No. 44
MEPGLWLLLGLTVTSAAGLVPCPQSGDSGRASVSQGPPEAGSERGCEETVAGP
GERIVSPTVALPAQPESAGQERAPGRSGKQEDKGLPAHHRPRR*CTCFTYKDK
ECVYYCHLDIIWINTPEQTVPYGLSNYRESLRGKR*SLGPVPESSQPSPWTRL
RCTCMGADDKACAHFCARTRDVTSYSGRAERPAAEEMRETGGPRQRLMSRTDK
AHRP
SEQ ID No. 45
Preproendothelin 3 mRNA from Mus musculus

| | |
|---|---|
| 1 | acagccggcc agccctgcgc agggatgggc agcgcgctct gaaagttcgt gaccgcctca |
| 61 | gccaagtaac tctgagccgg gacgcgcagc tcaggcagcg acaggactcg aaagctgtag |
| 121 | ccagtctcac tacccttttg cggtcacaag cggccaccct ccaggcccgg tgctcccgcg |
| 181 | cctgatcggg gttcatggag ccggggctgt ggctccttct cgggctcaca gtgacctccg |
| 241 | ctgcaggact tgtgccttgc ccccagtctg gggactctgg cagagccagt gtgtcccagg |
| 301 | gtccccctga agctggatca gagaggggct gtgaagagac tgtggctggc cctggtgaga |
| 361 | ggattgtgtc cccaacagtt gcactgcctg cacagcctga aagcgctggg caggaacggg |
| 421 | caccaggcag gtctgggaaa caagaggaca aggggctgcc tgcacaccac cgccctcgcc |
| 481 | gctgcacgtg cttcacttac aaggacaagg agtgtgtcta ctattgccac ctggacatca |
| 541 | tctggattaa cactcctgaa cagactgtgc cctatggact gtccaactac agagaaagcc |
| 601 | ttcggggaaa gaggtccttg gggccagttc cagaaagctc ccagccttct ccgtggacac |
| 661 | gcttgcgttg tacttgtatg ggggcggatg acaaggcctg tgcacacttc tgtgcacgca |
| 721 | ccagagatgt caccagttat tccgggagag cagaaaggcc agctgcagaa gagatgcggg |
| 781 | agactggagg cccacgtcaa aggttgatgt caaggacaga taaagcccac cggccttagc |
| 841 | tggatctaac aggccacaac tgatgcttct tgcttcctgc agtggacttc acctgctctc |
| 901 | cctgcctgcc cactcttcca ggaaagagcc ttggagcttg tgcatacaga gtttgaattt |
| 961 | ccacctcttt agcgacaagt tgggaattgg cctgaggcaa aaatgaaaga atgacccatc |
| 1021 | caaagagccc atgcttacct gtacaccctt accccaagaa tgcccaagtc caaaagaccc |
| 1081 | cagtttccct aatgggtaaa atgatcccac atgtgccctg ggggccggat gcctgaggct |
| 1141 | ccagtttcac aaggaaattg tttgagaggt atgttaagtc agaaagctac ttactggcct |
| 1201 | tggcatgact cctcttggag agtaagtgga caccaggctg gctctttaca aaagtaaaca |
| 1261 | cacatccatc tcctagaaga ttcaggacac ccatgtggcc ggagcaggcc ttcttctgca |
| 1321 | gcctgcgtgg cctgagcaag ttagcgaggc atctgtgtgc ttgagacctg gatcctagct |
| 1381 | gggcagtagt ctatgaaatt gaatttcata ggacttagaa atcttccact gtgcttaata |
| 1441 | ctcatcagaa gagcagctct caaaaacaag aaggaaaaga aaacaggaga tctagatgtt |
| 1501 | catgagacaa aaagagaaat aaacaacaag actcccccct cccccatatt cttgatggtt |
| 1561 | ttgaactctg gggcactcgg caaagagctg ggggttctgc gatggcctct gtggcagagc |
| 1621 | tcccaaccct gccttgcagt aagctgccct gaggggcagc ttcagctcaa ggctactggt |
| 1681 | ctggatatct gctttcatga ataaatgtgg atccttgggg agtggcttca aaataagccc |
| 1741 | aaaaacacaa actctttgta cattatgtaa atttctgttt gtctatataa ttggcaacaa |
| 1801 | ctgggaattg taacctctgt tcaaaatctc ttttagctga gctctttctt ctgtgtccgt |
| 1861 | ggtgagtatg ggggtggggg tgggatcaga ctgtgagttc ccatgtaaac tctactctgc |
| 1921 | aggcccagct gggagagtct gcccacctcc ccaccaggcc tcatagcaat gagaacctgt |
| 1981 | ctttgggcat gttccaaggg caccacgtgg agacacttta gctattgtgt gaagtggagc |
| 2041 | cttaaccaga tggtaaactt cagaggaccg tctcctaagt tattacaggt gtgaacgtgc |
| 2101 | ctgcttattg agggtgtgtg agagagtgct ggtttgctgg gggggaatgt acagttaaga |
| 2161 | gaagtaatta tttattgggg aactattttc tacgtaactc ctttatggga tctattaaaa |
| 2221 | gtaaaggcac tgtagataga tagatagaca gacagaaaaa tagattttaa attgtgttca |
| 2281 | aaaatccaaa cccatatctc ttagtagcta gaaattgttt aaatctcata agcactattt |
| 2341 | ggcacagtgg ccagattgta tttcaaaaac aaatccactc actgtgagaa cactcaggag |
| 2401 | ataagtcaaa tgcataataa aagaaaatct aaaagtttgc tctggcttgc aggcttttcc |
| 2461 | ttgcacacag ttacacattc actcttcaca ggcctctgga gaggacagga cagagccaga |
| 2521 | gttctggatg taggattcat ctagagagga aagtatagac caaggcgggt gggcagctat |
| 2581 | tgggaggaga ggagtttggg gaccatttga gaagataaac atcaaagtgt ctggaaaaga |
| 2641 | agaaggaggt ttggatgaag ttggtgactt ccttggaatg cctgctcttc tacacaacct |
| 2701 | tttcagggag acaaccatgg gcctattgct caaagcatct gagaattatc tccagaagtg |
| 2761 | atcacagtag caaggccaca caggacataa aagcaaatgg aagggaggct gtttgataaa |
| 2821 | agagggagag gggacaggag ggcagaggga gggcaggaga gggcaagggg atgaacattg |
| 2881 | ttaaagtatt aatgcaaatg ccattataaa agcatcactt tgtatgatcc atgctaataa |
| 2941 | acttttaaaa aagattctaa agc |

SEQ ID NO: 46
(GGGGS)3 linker
GGGGSGGGGSGGGGS

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An isolated polypeptide consisting of: (i) the amino acid sequence CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii);
wherein, optionally, the polypeptide is capable of one or more of:
inhibiting glucose production in hepatocytes,
promoting GLP-1 secretion in the rat perfused colon assay,
promoting GLP-1 secretion in GLUTag cells,
promoting glucose uptake in skeletal muscle cells, or
promoting glucose uptake in adipocytes.

2. A polypeptide comprising:
(a) a first polypeptide portion consisting of (i) the amino acid sequence CTCFTYKDKECVYYCHLDIIWINTPEQTVPYGLSNYRESL (SEQ ID No. 1), (ii) a fragment of (i) beginning at position 1 and ending at any one of positions 27 to 39 of SEQ ID No. 1, or (iii) an amino acid sequence having 1, 2, or 3 substitutions relative to the amino acid sequence set forth in (i) or (ii), and
(b) a second portion, which second portion is a polypeptide portion heterologous to said first polypeptide portion or is a detectable label;
wherein, optionally, the polypeptide is capable of one or more of:
inhibiting glucose production in hepatocytes,
promoting GLP-1 secretion in the rat perfused colon assay,
promoting GLP-1 secretion in GLUTag cells,
promoting glucose uptake in skeletal muscle cells, or
promoting glucose uptake in adipocytes.

3. The polypeptide of claim 1, wherein the substitution is a conservative substitution.

4. The polypeptide of claim 1, wherein the polypeptide consists of an amino acid sequence CTCFTYKDKECVYYCHLDIIWINTPEQTVPYGLSNYRES (SEQ ID No. 26), or an amino acid sequence having 1, 2, or 3 substitutions relative to SEQ ID No. 26.

5. The polypeptide of claim 2, wherein the first polypeptide portion consists of an amino acid sequence CTCFTYKDKECVYYCHLDIIWINTPEQTVPYGLSNYRES (SEQ ID No. 26), or an amino acid sequence having 1, 2, or 3 substitutions relative to SEQ ID No. 26.

6. The polypeptide of claim 1, wherein the polypeptide consists of an amino acid sequence having 1, 2, or 3 substitutions relative to the fragment of (i) beginning at position 1 and ending at position 27 of SEQ ID No. 1.

7. The polypeptide of claim 1, wherein the polypeptide consists of the fragment of (i) beginning at position 1 and ending at position 27 of SEQ ID No. 1.

8. The polypeptide of claim 2, wherein the first polypeptide portion consists of an amino acid sequence having 1, 2, or 3 substitutions relative to the fragment of (i) beginning at position 1 and ending at position 27 of SEQ ID No. 1.

9. The polypeptide of claim 2, wherein the first polypeptide portion consists of the fragment of (i) beginning at position 1 and ending at position 27 of SEQ ID No. 1.

10. The polypeptide of claim 1, wherein the polypeptide consists of an amino acid sequence having 1, 2, or 3 substitutions relative to the fragment of (i) beginning at position 1 and ending at position 31 of SEQ ID No. 1.

11. The polypeptide of claim 1, wherein the polypeptide consists of the fragment of (i) beginning at position 1 and ending at position 31 of SEQ ID No. 1.

12. The polypeptide of claim 2, wherein the first polypeptide portion consists of an amino acid sequence having 1, 2, or 3 substitutions relative to the fragment of (i) beginning at position 1 and ending at position 31 of SEQ ID No. 1.

13. The polypeptide of claim 2, wherein the first polypeptide portion consists of the fragment of (i) beginning at position 1 and ending at position 31 of SEQ ID No. 1.

14-15. (canceled)

16. The polypeptide of claim 1, wherein positions 1, 3, 11, and 15 of SEQ ID No. 1 are each C, and a first disulfide bridge connects the cysteine at position 1 of SEQ ID No. 1 with the cysteine at position 15 of SEQ ID No. 1, and a second disulfide bridge connects the cysteine at position 3 of SEQ ID No. 1 with the cysteine at position 11 of SEQ ID No.

17. An isolated polypeptide consisting of: (i) the amino acid sequence X1TX2FTYKDKEX3VYYX4HLDIIX5INTPEQTVPYGLSNYRX6SX7 (SEQ ID No. 2) or (ii) a fragment of (i) beginning at position 1 and ending at any one of positions 27 to 39 of SEQ ID No. 2;
wherein X1, X2, X3, X4, X5, X6, and X7 are independently selected from any amino acid; and
wherein, optionally, the polypeptide is capable of one or more of:
inhibiting glucose production in hepatocytes,
promoting GLP-1 secretion in the rat perfused colon assay,
promoting GLP-1 secretion in GLUTag cells,
promoting glucose uptake in skeletal muscle cells, or
promoting glucose uptake in adipocytes.

18. A polypeptide comprising:
(a) a first polypeptide portion consisting of: (i) X1TX2FTYKDKEX3VYYX4HLDIIX5INTPEQTVPYGLSNYRX6SX7 (SEQ ID No. 2) or (ii) a fragment of (i) beginning at position 1 and ending at any one of positions 27 to 39 SEQ ID No. 2 and
(b) a second portion, which polypeptide portion is a polypeptide portion heterologous to said first polypeptide portion or is a detectable label;
wherein X1, X2, X3, X4, X5, X6, and X7 are independently selected from any amino acid; and
wherein, optionally, the polypeptide is capable of one or more of:
inhibiting glucose production in hepatocytes,
promoting GLP-1 secretion in the rat perfused colon assay,
promoting GLP-1 secretion in GLUTag cells,
promoting glucose uptake in skeletal muscle cells, or
promoting glucose uptake in adipocytes.

19. The polypeptide of claim 17 or 18, wherein X1, X2, X3, X4, X5, X6, and X7 are independently selected from the corresponding position in SEQ ID NO: 19 or SEQ ID NO: 21 or a conservative substitution thereof.

20. The polypeptide of claim 17, wherein the polypeptide consists of an amino acid sequence X1TX2FTYKDKEX3VYYX4HLDIIX5INTPEQTVPYGLSNYRX6S (SEQ ID No. 27), wherein X1, X2, X3, X4, X5, and X6 are independently selected from any amino acid.

21. The polypeptide of claim 18, wherein the first polypeptide portion consists of an amino acid sequence X1 TX2FTYKDKEX3VYYX4HLDIIX5INTPEQTVPYGLSNYRX6S (SEQ ID No. 27), wherein X1, X2, X3, X4, X5, and X6 are independently selected from any amino acid.

22-195. (canceled)


SEQUENCE LISTING (46 sequences)

SEQ ID NO: 1 - protein, 40 aa, Homo sapiens
CTCFTYKDKECVYYCHLDIIWINTPEQTVPYGLSNYRESL

SEQ ID NO: 2 - protein, 40 aa, Homo sapiens
  misc_feature (1)..(1): Xaa can be any naturally occurring amino acid
XTXFTYKDKEXVYYXHLDIIXINTPEQTVPYGLSNYRXSX

SEQ ID NO: 3 - protein, 15 aa, Homo sapiens
YYSHLDIIWINTPEQ

SEQ ID NO: 4 - protein, 15 aa, Homo sapiens
YYAHLDIIAINTPEQ

SEQ ID NO: 5 - protein, 29 aa, Homo sapiens
  misc_feature (26)..(26): Xaa can be any naturally occurring amino acid
YYSHLDIIWINTPEQTVPYGLSNYRXSXR

SEQ ID NO: 6 - protein, 29 aa, Homo sapiens
  misc_feature (26)..(26): Xaa can be any naturally occurring amino acid
YYAHLDIIAINTPEQTVPYGLSNYRXSXR

SEQ ID NO: 7 - protein, 15 aa, Homo sapiens
YYCHLDIIWINTPEQ

SEQ ID NO: 8 - protein, 15 aa, Homo sapiens
  misc_feature (3)..(3): Xaa can be any naturally occurring amino acid
YYXHLDIIXINTPEQ

SEQ ID NO: 9 - protein, 27 aa, Homo sapiens
CTCFTYKDKECVYYCHLDIIWINTPEQ

SEQ ID NO: 10 - protein, 27 aa, Homo sapiens
  misc_feature (1)..(1): Xaa can be any naturally occurring amino acid
XTXFTYKDKEXVYYXHLDIIXINTPEQ

SEQ ID NO: 11 - protein, 31 aa, Homo sapiens
CTCFTYKDKECVYYCHLDIIWINTPEQTVPY

SEQ ID NO: 12 - protein, 31 aa, Homo sapiens
  misc_feature (1)..(1): Xaa can be any naturally occurring amino acid
XTXFTYKDKEXVYYXHLDIIXINTPEQTVPY

SEQ ID NO: 13 - protein, 41 aa, Homo sapiens
CTCFTYKDKECVYYCHLDIIWINTPEQTVPYGLSNYRGSFR

SEQ ID NO: 14 - protein, 41 aa, Homo sapiens
  misc_feature (1)..(1): Xaa can be any naturally occurring amino acid
XTXFTYKDKEXVYYXHLDIIXINTPEQTVPYGLSNYRXSXR

SEQ ID NO: 15 - protein, 29 aa, Homo sapiens
YYCHLDIIWINTPEQTVPYGLSNYRGSFR

SEQ ID NO: 16 - protein, 29 aa, Homo sapiens
  misc_feature (3)..(3): Xaa can be any naturally occurring amino acid
YYXHLDIIXINTPEQTVPYGLSNYRXSXR

SEQ ID NO: 17 - protein, 32 aa, Homo sapiens
YYCHLDIIWINTPEQTVPYGLSNYRGSFRGKR

SEQ ID NO: 18 - protein, 32 aa, Homo sapiens
  misc_feature (3)..(3): Xaa can be any naturally occurring amino acid
YYXHLDIIXINTPEQTVPYGLSNYRXSXRGKR

SEQ ID NO: 19 - protein, 44 aa, Homo sapiens
CTCFTYKDKECVYYCHLDIIWINTPEQTVPYGLSNYRGSFRGKR

SEQ ID NO: 20 - protein, 44 aa, Homo sapiens
  misc_feature (1)..(1): Xaa can be any naturally occurring amino acid
XTXFTYKDKEXVYYXHLDIIXINTPEQTVPYGLSNYRXSXRGKR

SEQ ID NO: 21 - protein, 44 aa, Homo sapiens
CTCFTYKDKECVYYCHLDIIWINTPEQTVPYGLSNYRESLRGKR

SEQ ID NO: 22 - protein, 32 aa, Homo sapiens
  misc_feature (26)..(26): Xaa can be any naturally occurring amino acid
YYSHLDIIWINTPEQTVPYGLSNYRXSXRGKR

SEQ ID NO: 23 - protein, 32 aa, Homo sapiens
  misc_feature (26)..(26): Xaa can be any naturally occurring amino acid
YYAHLDIIAINTPEQTVPYGLSNYRXSXRGKR

SEQ ID NO: 24 - protein, 22 aa, Homo sapiens
YKDKECVYYCHLDIIWINTPEQ

SEQ ID NO: 25 - protein, 22 aa, Homo sapiens
  misc_feature (6)..(6): Xaa can be any naturally occurring amino acid
YKDKEXVYYXHLDIIXINTPEQ

SEQ ID NO: 26 - protein, 39 aa, Homo sapiens
CTCFTYKDKECVYYCHLDIIWINTPEQTVPYGLSNYRES

SEQ ID NO: 27 - protein, 39 aa, Homo sapiens
  misc_feature (1)..(1): Xaa can be any naturally occurring amino acid
XTXFTYKDKEXVYYXHLDIIXINTPEQTVPYGLSNYRXS

SEQ ID NO: 28 - protein, 39 aa, Homo sapiens
CTCFTYKDKECVYYCHLDIIWINTPEQTVPYGLSNYRGS

SEQ ID NO: 29 - protein, 41 aa, Homo sapiens
CTCFTYKDKECVYYCHLDIIWINTPEQTVPYGLSNYRESLR

SEQ ID NO: 30 - protein, 44 aa, Homo sapiens
CTCFTYKDKECVYYCHLDIIFINTPEQTVPYGLSNYRESLRGKR

SEQ ID NO: 31 - protein, 44 aa, Homo sapiens
ATCFTYKDKECVYYAHLDIIWINTPEQTVPYGLSNYRESLRGKR

SEQ ID NO: 32 - protein, 44 aa, Homo sapiens
CTAFTYKDKEAVYYCHLDIIWINTPEQTVPYGLSNYRESLRGKR

SEQ ID NO: 33 - protein, 28 aa, Homo sapiens
  misc_feature (1)..(1): Xaa can be any naturally occurring amino acid
XTXFTYKDKEXVYYXHLDIIXINTPEQT

SEQ ID NO: 34 - protein, 43 aa, Homo sapiens
TCFTYKDKECVYYCHLDIIWINTPEQTVPYGLSNYRESLRGKR

SEQ ID NO: 35 - protein, 41 aa, Homo sapiens
FTYKDKECVYYCHLDIIWINTPEQTVPYGLSNYRESLRGKR

SEQ ID NO: 36 - protein, 11 aa, Homo sapiens
FTYKDKESVYY

SEQ ID NO: 37 - protein, 32 aa, Homo sapiens
YYSHLDIIWINTPEQTVPYGLSNYRESLRGKR

SEQ ID NO: 38 - protein, 32 aa, Homo sapiens
YYAHLDIIAINTPEQTVPYGLSNYRESLRGKR

SEQ ID NO: 39 - protein, 23 aa, Homo sapiens
INTPEQTVPYGLSNYRESLRGKR

SEQ ID NO: 40 - protein, 21 aa, Homo sapiens
CTCFTYKDKECVYYCHLDIIW

SEQ ID NO: 41 - protein, 22 aa, Homo sapiens
YKDKECVYYCHLDIIWINTPEQ

SEQ ID NO: 42 - protein, 44 aa, Homo sapiens
STSFTYKDKESVYYSHLDIIWINTPEQTVPYGLSNYRESLRGKR

SEQ ID NO: 43 - protein, 44 aa, Homo sapiens
CTCFTYKDKECVYYCHLDIIHINTPEQTVPYGLSNYRESLRGKR

SEQ ID NO: 44 - protein, 214 aa, Homo sapiens
MEPGLWLLLGLTVTSAAGLVPCPQSGDSGRASVSQGPPEAGSERGCEETVAGPGERIVSPTVALPAQPESAGQERAPGRSGKQEDKGLPAHHRPRRCTCFTYKDKECVYYCHLDIIWINTPEQTVPYGLSNYRESLRGKRSLGPVPESSQPSPWTRLRCTCMGADDKACAHFCARTRDVTSYSGRAERPAAEEMRETGGPRQRLMSRTDKAHRP

SEQ ID NO: 45 - DNA, 2963 bp, Mus musculus
acagccggccagccctgcgcagggatgggcagcgcgctctgaaagttcgtgaccgcctcagccaagtaactctgagccgggacgcgcagctcaggcagcgacaggactcgaaagctgtagccagtctcactacccttttgcggtcacaagcggccaccctccaggcccggtgctcccgcgcctgatcggggttcatggagccggggctgtggctccttctcgggctcacagtgacctccgctgcaggacttgtgccttgcccccagtctggggactctggcagagccagtgtgtcccagggtccccctgaagctggatcagagaggggctgtgaagagactgtggctggccctggtgagaggattgtgtccccaacagttgcactgcctgcacagcctgaaagcgctgggcaggaacgggcaccaggcaggtctgggaaacaagaggacaaggggctgcctgcacaccaccgccctcgccgctgcacgtgcttcacttacaaggacaaggagtgtgtctactattgccacctggacatcatctggattaacactcctgaacagactgtgccctatggactgtccaactacagagaaagccttcggggaaagaggtccttggggccagttccagaaagctcccagccttctccgtggacacgcttgcgttgtacttgtatgggggcggatgacaaggcctgtgcacacttctgtgcacgcaccagagatgtcaccagttattccgggagagcagaaaggccagctgcagaagagatgcgggagactggaggcccacgtcaaaggttgatgtcaaggacagataaagcccaccggccttagctggatctaacaggccacaactgatgcttcttgcttcctgcagtggacttcacctgctctccctgcctgcccactcttccaggaaagagccttggagcttgtgcatacagagtttgaatttccacctctttagcgacaagttgggaattggcctgaggcaaaaatgaaagaatgacccatccaaagagcccatgcttacctgtacacccttaccccaagaatgcccaagtccaaaagaccccagtttccctaatgggtaaaatgatcccacatgtgccctgggggccggatgcctgaggctccagtttcacaaggaaattgtttgagaggtatgttaagtcagaaagctacttactggccttggcatgactcctcttggagagtaagtggacaccaggctggctctttacaaaagtaaacacacatccatctcctagaagattcaggacacccatgtggccggagcaggccttcttctgcagcctgcgtggcctgagcaagttagcgaggcatctgtgtgcttgagacctggatcctagctgggcagtagtctatgaaattgaatttcataggacttagaaatcttccactgtgcttaatactcatcagaagagcagctctcaaaaacaagaaggaaaagaaaacaggagatctagatgttcatgagacaaaaagagaaataaacaacaagactcccccctcccccatattcttgatggttttgaactctggggcactcggcaaagagctgggggttctgcgatggcctctgtggcagagctcccaaccctgccttgcagtaagctgccctgaggggcagcttcagctcaaggctactggtctggatatctgctttcatgaataaatgtggatccttggggagtggcttcaaaataagcccaaaaacacaaactctttgtacattatgtaaatttctgtttgtctatataattggcaacaactgggaattgtaacctctgttcaaaatctcttttagctgagctctttcttctgtgtccgtggtgagtatgggggtgggggtgggatcagactgtgagttcccatgtaaactctactctgcaggcccagctgggagagtctgcccacctccccaccaggcctcatagcaatgagaacctgtctttgggcatgttccaagggcaccacgtggagacactttagctattgtgtgaagtggagccttaaccagatggtaaacttcagaggaccgtctcctaagttattacaggtgtgaacgtgcctgcttattgagggtgtgtgagagagtgctggtttgctgggggggaatgtacagttaagagaagtaattatttattggggaactattttctacgtaactcctttatgggatctattaaaagtaaaggcactgtagatagatagatagacagacagaaaaatagattttaaattgtgttcaaaaatccaaacccatatctcttagtagctagaaattgtttaaatctcataagcactatttggcacagtggccagattgtatttcaaaaacaaatccactcactgtgagaacactcaggagataagtcaaatgcataataaaagaaaatctaaaagtttgctctggcttgcaggcttttccttgcacacagttacacattcactcttcacaggcctctggagaggacaggacagagccagagttctggatgtaggattcatctagagaggaaagtatagaccaaggcgggtgggcagctattgggaggagaggagtttggggaccatttgagaagataaacatcaaagtgtctggaaaagaagaaggaggtttggatgaagttggtgacttccttggaatgcctgctcttctacacaaccttttcagggagacaaccatgggcctattgctcaaagcatctgagaattatctccagaagtgatcacagtagcaaggccacacaggacataaaagcaaatggaagggaggctgtttgataaaagagggagaggggacaggagggcagagggagggcaggagagggcaaggggatgaacattgttaaagtattaatgcaaatgccattataaaagcatcactttgtatgatccatgctaataaacttttaaaaaagattctaaagc

SEQ ID NO: 46 - protein, 15 aa, Homo sapiens
GGGGSGGGGSGGGGS